(19) United States
(12) Patent Application Publication
(10) Pub. No.: US 2023/0093512 A1
(43) Pub. Date: Mar. 23, 2023

(54) FUSION PROTEINS AND USES THEREOF

(71) Applicant: Nanjing GenScript Biotech Co., Ltd., Nanjing (CN)

(72) Inventors: Zhongdao Li, Nanjing (CN); Liusong Yin, Nanjing (CN); Tielin Zhou, Singapore (SG); Tianhui Xia, Nanjing (CN); Zhuo Fang, Nanjing (CN); Yi Jin, Nanjing (CN)

(73) Assignee: Nanjing GenScript Biotech Co., Ltd., Nanjing (CN)

(21) Appl. No.: 17/799,890

(22) PCT Filed: Feb. 18, 2021
(86) PCT No.: PCT/CN2021/076705

§ 371 Date: Aug. 15, 2022

(30) Foreign Application Priority Data
Feb. 18, 2020 (CN) PCT/CN2020/075666

(51) Int. Cl. C07K 16/28; C07K 14/71

(52) U.S. Cl. CPC C07K 16/2827; C07K 14/71; C07K 2317/92; C07K 2319/02; C07K 2319/33; A61K 38/00

(57) ABSTRACT

The present application provides fusion proteins that have a TGFβ superfamily receptor ectodomain (such as fusion proteins that have a TGFβ superfamily receptor ectodomain and bind to PD-L1), nucleic acids encoding the fusion protein or a portion thereof, vectors comprising the nucleic acids, host cells containing the vectors, methods of preparing the fusion proteins, pharmaceutical compositions comprising any of the fusion proteins, and methods of using the fusion proteins or compositions.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 759892000640SEQLIST.TXT, date recorded: Feb. 14, 2020, size: 125 KB).

### FIELD OF THE APPLICATION

The present application relates to fusion proteins that comprise one or more TGFβ superfamily receptor ectodomain and bind to PD-L1, methods of making, and uses thereof including treating diseases or conditions.

### BACKGROUND OF THE APPLICATION

In cancer treatment, it has long been recognized that chemotherapy is associated with high toxicity and can lead to emergence of resistant cancer cell variants. Even with targeted therapy against overexpressed or activated oncoproteins important for tumor survival and growth, cancer cells invariably mutate and adapt to reduce dependency on the targeted pathway, such as by utilizing a redundant pathway. Cancer immunotherapy is a new paradigm in cancer treatment that instead of targeting cancer cells, focuses on the activation of the immune system. Its principle is to rearm the host's immune response, especially the adaptive T cell response, to provide immune surveillance to kill the cancer cells, in particular, the minimal residual disease that has escaped other forms of treatment, hence achieving long-lasting protective immunity.

Programmed death 1 (PD-1) is a member of the CD28 superfamily. PD-1 is expressed in activated T cells, B cells and myeloid cells, which have two ligands, programmed death ligand 1, PD-L1 and PD-L2. PD-L1 interacts with receptor PD-1 on T cells and plays an important role in the negative regulation of immune responses. The expression of PD-L1 protein can be detected in many human tumor tissues. The microenvironment of the tumor site can induce the expression of PD-L1 on tumor cells. The expression of PD-L1 is beneficial to the occurrence and growth of tumors, and induces anti-tumor. Anti-PD-1 or anti-PD-L1 therapy have shown encouraging results in melanoma, kidney, and lung cancer in clinical trials. However, these therapies are also limited by their toxicity profile and limited therapeutic effects. For example, inhibiting the PD-L1/PD-1 interaction results in dis-inhibiting existing chronic immune responses in exhausted T cells that are mostly antiviral or anticancer in nature (Wherry, E. J., Nat Immunol. 2011; 12:492-9), anti-PD-1 therapy can nevertheless sometimes result in potentially fatal lung-related autoimmune adverse events.

The disclosures of all publications, patents, patent applications and published patent applications referred to herein are hereby incorporated herein by reference in their entirety.

### BRIEF SUMMARY OF THE APPLICATION

The present application provides fusion proteins comprising a TGFβ superfamily receptor ectodomain. In some embodiments, the fusion proteins comprise a) a full-length antibody that specifically binds to PD-L1 and comprises two heavy chains and two light chains, wherein each of the two heavy chains comprises a heavy chain variable region (V_{H}) and each of the two light chains comprises a light chain variable region (V_{L}); b) a TGFβ superfamily receptor ectodomain.

In one aspect, the present application provides a fusion protein comprising: a) a full-length antibody that specifically binds to PD-L1 and comprises two heavy chains and two light chains, wherein each of the two heavy chains comprises a heavy chain variable region (V_{H}) and each of the two light chains comprises a light chain variable region (V_{L}); b) a TGFβ superfamily receptor ectodomain; wherein the TGFβ superfamily receptor ectodomain is fused to at least one of the heavy chains or light chains of the anti-PD-L1 antibody. In some embodiments, the TGFβ superfamily receptor ectodomain is fused to the at least one or both of the two light chains. In some embodiments, the TGFβ superfamily receptor ectodomain is fused to the C-terminus of the at least one or both of the two light chains. In some embodiments, the TGFβ superfamily receptor ectodomain is fused to the N-terminus of the at least one or both of the two light chains. In some embodiments, the TGFβ superfamily receptor ectodomain is fused to the at least one or both of the two heavy chains. In some embodiments, the TGFβ superfamily receptor ectodomain is fused to the C-terminus of the at least one or both of the two heavy chains. In some embodiments, the TGFβ superfamily receptor ectodomain is fused to the N-terminus of at least one or both of the two heavy chains.

In some embodiments of any one of the fusion proteins described above, the TGFβ superfamily receptor ectodomain comprises a TGFβ receptor type II ectodomain or a variant thereof. In some embodiments, the TGFβ receptor type II ectodomain or a variant thereof comprises a human TGFβ receptor type II ectodomain or a variant thereof. In some embodiments, the human TGFβ receptor type II ectodomain or a variant thereof comprises a variant that comprises an amino acid sequence that has at least about 90% sequence identity to SEQ ID NO: 2, wherein the variant comprises one or more amino acid substitution at position N47, N71, and/or N131 according to the numbering of SEQ ID NO: 2. In some embodiments, the variant comprises the amino acid sequence of SEQ ID NO: 3.

In some embodiments of any one of the fusion proteins described above, the TGFβ superfamily receptor ectodomain comprises a TGFβ receptor type I ectodomain or a variant thereof. In some embodiments, the TGFβ receptor type I ectodomain or a variant thereof comprises a human TGFβ receptor type I ectodomain or a variant thereof.

In some embodiments of any one of the fusion proteins described above, the V_{H} comprises: i) a HC-CDR1 comprising an amino acid sequence of SEQ ID NO: 6; ii) a HC-CDR2 comprising an amino acid sequence of SEQ ID NO: 7; and iii) a HC-CDR3 comprising an amino acid sequence of SEQ ID NO: 8.

In some embodiments of any one of the fusion proteins described above, the V_{L} comprises: i) a LC-CDR1 comprising an amino acid sequence of SEQ ID NO: 9; ii) a LC-CDR2 comprising an amino acid sequence of SEQ ID NO: 10; and iii) a LC-CDR3 comprising an amino acid sequence of SEQ ID NO: 11.

In some embodiments of any one of the fusion proteins described above, the TGFβ superfamily receptor ectodomain is fused to at least one of the heavy chains or light chains of the anti-PD-L1 antibody via a linker. In some embodiments, the linker is a peptide linker. In some embodiments, the peptide linker has a length of about 2 to about 25 amino acids. In some embodiments, the peptide linker has a length of about 2 to about 10 amino acids. In some embodiments, the linker is a GS linker. In some embodiments, the linker comprises a modified sequence derived from the hinge region of an IgG. In some embodiments, the linker comprises the amino acid sequence of SEQ ID NOs: 56-68.

In some embodiments of any one of the fusion proteins described above, the V_{H} comprises the amino acid sequence of SEQ ID NO: 12 or a variant thereof having at least about 80% (including for example at least about any of 80%, 85%, 90%, 95%, 96%, 97%, 98%, or 99%) sequence identity, and wherein the V_{L} comprises the amino acid sequence of SEQ ID NO: 13 or a variant thereof having at least about 80% (including for example at least about any of 80%, 85%, 90%, 95%, 96%, 97%, 98%, or 99%) sequence identity.

In some embodiments of any one of the fusion proteins described above, the heavy chain comprises the amino acid sequence of any one of SEQ ID NOs: 14-17 or a variant thereof having at least about 80% (including for example at least about any of 80%, 85%, 90%, 95%, 96%, 97%, 98%, or 99%) sequence identity, and wherein the light chain comprises the amino acid sequence of SEQ ID NO: 18 or a variant thereof having at least about 80% (including for example at least about any of 80%, 85%, 90%, 95%, 96%, 97%, 98%, or 99%) sequence identity. In some embodiments, the heavy chain comprises the amino acid sequence of SEQ ID NO: 16 or 17, and wherein the light chain comprises the amino acid sequence of SEQ ID NO: 18.

In some embodiments of any one of the fusion proteins described above, the TGFβ superfamily receptor ectodomain or the polypeptide fused to at least one or both of the two light chains comprises the amino acid sequence of any one of SEQ ID NOs: 27-38 or a variant thereof having at least about 80% (including for example at least about any of 80%, 85%, 90%, 95%, 96%, 97%, 98%, or 99%) sequence identity.

In some embodiments of any one of the fusion proteins described above, the TGFβ superfamily receptor ectodomain fused to at least one or both of the two heavy chains comprises the amino acid sequence of any one of SEQ ID NOs: 22-26 or a variant thereof having at least about 80% (including for example at least about any of 80%, 85%, 90%, 95%, 96%, 97%, 98%, or 99%) sequence identity.

In some embodiments of any one of the fusion proteins described above, the TGFβ superfamily receptor ectodomain is fused to both of the two heavy chains comprising the amino acid sequence of SEQ ID NO: 22, and the two light chains comprise the amino acid sequence of SEQ ID NO: 18.

In some embodiments of any one of the fusion proteins described above, the TGFβ superfamily receptor ectodomain is fused to both of the two heavy chains comprising the amino acid sequence of SEQ ID NO: 23, and the two light chains comprise the amino acid sequence of SEQ ID NO: 18.

In some embodiments of any one of the fusion proteins described above, the TGFβ superfamily receptor ectodomain is fused to both of the two light chains comprising the amino acid sequence of SEQ ID NO: 27, and both of the two heavy chains comprises the amino acid sequence of SEQ ID NO: 16 or 17.

In some embodiments of any one of the fusion proteins described above, the TGFβ superfamily receptor ectodomain is fused to both of the two light chains comprising the amino acid sequence of SEQ ID NO: 28, and both of the two heavy chains comprises the amino acid sequence of SEQ ID NO: 16 or 17.

In some embodiments of any one of the fusion proteins described above, the TGFβ superfamily receptor ectodomain is fused to both of the two heavy chains comprising the amino acid sequence of SEQ ID NO: 22, and the TGFβ superfamily receptor ectodomain is also fused to both of the two light chains comprising the amino acid sequence of SEQ ID NO: 28.

In some embodiments of any one of the fusion proteins described above, the TGFβ superfamily receptor ectodomain is fused to both of the two heavy chains comprising the amino acid sequence of SEQ ID NO: 23, and the TGFβ superfamily receptor ectodomain is also fused to both of the two light chains comprising the amino acid sequence of SEQ ID NO: 27.

In some embodiments of any one of the fusion proteins described above, the TGFβ superfamily receptor ectodomain is fused to both of the two heavy chains comprising the amino acid sequence of SEQ ID NO: 22, and the TGFβ superfamily receptor ectodomain is also fused to both of the two light chains comprising the amino acid sequence of SEQ ID NO: 27.

In some embodiments of any one of the fusion proteins described above, the TGFβ superfamily receptor ectodomain is fused to both of the two heavy chains comprising the amino acid sequence of SEQ ID NO: 23, and the TGFβ superfamily receptor ectodomain is also fused to both of the two light chains comprising the amino acid sequence of SEQ ID NO: 28.

In some embodiments of any one of the fusion proteins described above, the polypeptide is fused to both of the two heavy chains comprising the amino acid sequence of SEQ ID NO: 24, and both of the two light chains comprise the amino acid sequence of SEQ ID NO: 18.

In some embodiments of any one of the fusion proteins described above, the TGFβ superfamily receptor ectodomain is fused to both of the two heavy chains comprising the amino acid sequence of SEQ ID NO: 25, and both of the two light chains comprise the amino acid sequence of SEQ ID NO: 18.

In some embodiments of any one of the fusion proteins described above, the TGFβ superfamily receptor ectodomain is fused to both of the two heavy chains comprising the amino acid sequence of SEQ ID NO: 26, and both of the two light chains comprise the amino acid sequence of SEQ ID NO: 18.

In some embodiments of any one of the fusion proteins described above, the TGFβ superfamily receptor ectodomain is fused to both of the two light chains comprising the amino acid sequence of SEQ ID NO: 29, and both of the two heavy chains comprise the amino acid sequence of SEQ ID NO: 16 or 17.

In some embodiments of any one of the fusion proteins described above, the TGFβ superfamily receptor ectodomain is fused to both of the two light chains comprising the amino acid sequence of SEQ ID NO: 30, and both of the two heavy chains comprise the amino acid sequence of SEQ ID NO: 16 or 17.

In some embodiments of any one of the fusion proteins described above, the TGFβ superfamily receptor ectodomain is fused to both of the two light chains comprising the amino acid sequence of SEQ ID NO: 31, and both of the two heavy chains comprise the amino acid sequence of SEQ ID NO: 16 or 17.

In some embodiments of any one of the fusion proteins described above, the TGFβ superfamily receptor ectodomain is fused to both of the two light chains comprising the amino acid sequence of SEQ ID NO: 32, and both of the two heavy chains comprise the amino acid sequence of SEQ ID NO: 16 or 17.

In some embodiments of any one of the fusion proteins described above, the TGFβ superfamily receptor ectodomain is fused to both of the two light chains comprising the amino acid sequence of SEQ ID NO: 33, and both of the two heavy chains comprise the amino acid sequence of SEQ ID NO: 16 or 17.

In some embodiments of any one of the fusion proteins described above, the TGFβ superfamily receptor ectodomain is fused to both of the two light chains comprising the amino acid sequence of SEQ ID NO: 34, and both of the two heavy chains comprise the amino acid sequence of SEQ ID NO: 16 or 17.

In some embodiments of any one of the fusion proteins described above, the TGFβ superfamily receptor ectodomain is fused to both of the two light chains comprising the amino acid sequence of SEQ ID NO: 35 and both of the two heavy chains comprise the amino acid sequence of SEQ ID NO: 16 or 17.

In some embodiments of any one of the fusion proteins described above, the TGFβ superfamily receptor ectodomain is fused to both of the two light chains comprising the amino acid sequence of SEQ ID NO: 36 and both of the two heavy chains comprise the amino acid sequence of SEQ ID NO: 16 or 17.

In some embodiments of any one of the fusion proteins described above, the TGFβ superfamily receptor ectodomain is fused to both of the two light chains comprising the amino acid sequence of SEQ ID NO: 37 and both of the two heavy chains comprise the amino acid sequence of SEQ ID NO: 16 or 17.

In another aspect, the present application provides a polypeptide comprising a variant human TGFβ receptor type II ectodomain that has at least about 90% (including for example at least about any of 90%, 91%, 92%, 93%, 94%, 95%, 96%, 97%, 98%, or 99%) sequence identity to SEQ ID NO: 2, wherein the variant comprises one or more amino acid substitution at position N47, N71, and/or N131 according to the numbering of SEQ ID NO: 2. In some embodiments, the variant comprises the amino acid sequence of SEQ ID NO: 3.

In another aspect, the present application provides a fusion protein comprising a) any of the polypeptides described above; and b) a second moiety. In some embodiments, the second moiety comprises a half-life extending domain. In some embodiments, the second moiety comprises an Fc fragment. In some embodiments, the second moiety comprises an immune checkpoint inhibitor. In some embodiments, the immune checkpoint inhibitor comprises an anti-PD-L1 antibody moiety.

In another aspect, the present application provides a pharmaceutical composition comprising any of the fusion proteins or polypeptides.

In another aspect, the present application provides a nucleic acid encoding any of the fusion proteins or a portion thereof, or any of the polypeptides described above.

In another aspect, the present application provides a nucleic acid comprising the nucleic acid sequence selected from the group consisting of SEQ ID NOs: 19-21 and 38-53.

In another aspect, the present application provides a vector comprising any of the nucleic acids described above.

In another aspect, the present application provides a host cell comprising any of the nucleic acids or the vectors described above.

In another aspect, the present application provides a method of producing any of the fusion proteins or polypeptides described above, comprising: a) culturing the host cell of described above under conditions effective to express the fusion protein or the polypeptide; and b) obtaining the expressed fusion protein or the polypeptide from the host cell.

In another aspect, the present application provides a method of treating a disease or condition in an individual, comprising administering to the individual an effective amount of any one of the fusion proteins or the pharmaceutical compositions described above. In some embodiments, the disease or condition is a cancer. In some embodiments, the cancer is non-small cell lung cancer (NSCLC), small cell lung cancer (SCLC), cervical cancer, oropharyngeal cancer, anal cancer, vaginal or penile cancer, biliary tract cancer, cholangiocarcinoma, gallbladder cancer, head and neck cancer, pancreas cancer, prostate cancer, urothelial cancer, bladder cancer, genitourinary cancer, urogenital cancer, gastric cancer, breast cancer or colorectal cancer. In some embodiments, the cancer is advanced or metastatic cancer. In some embodiments, the method further comprises administering a second agent or therapy. In some embodiments, the fusion protein or the pharmaceutical composition is administered parenterally into the individual. In some embodiments, the individual is a human.

In another aspect, the present application provides a kit comprising any one of the pharmaceutical compositions and an instruction for treating a disease or condition.

### BRIEF DESCRIPTION OF THE DRAWINGS

FIG. 1 shows binding affinities of various fusion proteins to TGFβ1.

FIG. 2 shows binding affinities of various fusion proteins to TGFβ2.

FIG. 3 shows binding affinities of various fusion proteins to TGFβ3.

FIG. 4 shows binding affinities of various fusion proteins to PD-L1.

FIG. 5 shows results of PD-1/PD-L1 blockage assay as illustrated in Example 2.

FIG. 6 shows binding affinities of various fusion proteins to TGFβ1.

FIG. 7 shows binding affinities of various fusion proteins to TGFβ2.

FIG. 8 shows binding affinities of various fusion proteins to TGFβ3.

FIG. 9 shows binding affinities of various fusion proteins to PD-L1.

FIG. 10 shows results of PD-1/PD-L1 blockage assay as illustrated in Example 2.

FIG. 11 shows results of PD-1/PD-L1 blockage assay as illustrated in Example 2.

### DETAILED DESCRIPTION OF THE APPLICATION

The present application provides fusion proteins that comprise one or more TGFβ superfamily receptor ectodomain. In some embodiments, the fusion protein comprises a) an anti-PD-L1 full-length antibody and b) a TGFβ superfamily receptor ectodomain (such as a type I or type II ectodomain or a variant thereof). In some embodiments, the TGFβ superfamily receptor ectodomain is fused to C-terminus of the one or both of the light chains of the anti-PD-L1 full-length antibody. In some embodiments, the anti-PD-L1 full-length antibody comprises a) two heavy chains each comprising a heavy chain variable region (V_{H}) comprising: i) a HC-CDR1 comprising an amino acid sequence of SEQ ID NO: 6; ii) a HC-CDR2 comprising an amino acid sequence of SEQ ID NO: 7; and iii) a HC-CDR3 comprising an amino acid sequence of SEQ ID NO: 8, and b) two light chains each comprising a light chain variable region (V_{L}) comprising: i) a LC-CDR1 comprising an amino acid sequence of SEQ ID NO: 9; ii) a LC-CDR2 comprising an amino acid sequence of SEQ ID NO: 10; and iii) a LC-CDR3 comprising an amino acid sequence of SEQ ID NO: 11. The present application also provides a variant human TGFβ receptor type II ectodomain comprising an amino sequence that has at least about 90% sequence identity to SEQ ID NO: 2, wherein the variant comprises one or more amino acid substitution at position N47, N71, and/or N131 according to the numbering of SEQ ID NO: 2. The present application also provides methods of preparing the fusion proteins or polypeptides described herein and methods of treatment.

The present application is at least partly based upon the advantageous effects exhibited by the fusion proteins described herein (such as those that comprise an anti-PD-L1 full-length antibody and a TGFβ type II receptor fused to the light chains of the anti-PD-L1 antibody) as compared to reference fusion protein (such as M7824). Specifically, the fusion proteins described herein have shown lower binding affinity to TGFβ2, which plays physiological roles in normal cardiac function and early hematopoiesis, thereby reducing the toxicity effects. See, for example, Bhandary et al., J Am Heart Assoc. 2018 Oct. 16; 7(20):e010013.

### Definitions

Techniques and procedures described or referenced herein include those that are generally well understood and/or commonly employed using conventional methodology by those skilled in the art, such as, for example, the widely utilized methodologies described in Sambrook et al., Molecular Cloning: A Laboratory Manual (3d ed. 2001); Current Protocols in Molecular Biology (Ausubel et al. eds., 2003); Therapeutic Monoclonal Antibodies: From Bench to Clinic (An ed. 2009); Monoclonal Antibodies: Methods and Protocols (Albitar ed. 2010); and Antibody Engineering Vols 1 and 2 (Kontermann and Dithel eds., 2d ed. 2010).

Unless otherwise defined herein, technical and scientific terms used in the present description have the meanings that are commonly understood by those of ordinary skill in the art. For purposes of interpreting this specification, the following description of terms will apply and whenever appropriate, terms used in the singular will also include the plural and vice versa. In the event that any description of a term set forth conflicts with any document incorporated herein by reference, the description of the term set forth below shall control.

“Binding affinity” generally refers to the strength of the sum total of noncovalent interactions between a single binding site of a molecule (e.g., a binding protein such as an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a binding molecule X for its binding partner Y can generally be represented by the dissociation constant (K_{D}). Affinity can be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present disclosure. Specific illustrative embodiments include the following. In one embodiment, the “K_{D}” or “K_{D} value” may be measured by assays known in the art, for example by a binding assay. The K_{D} may be measured in a RIA, for example, performed with the Fab version of an antibody of interest and its antigen (Chen et al., 1999, J. Mol Biol 293:865-81). The K_{D} or K_{D} value may also be measured by using biolayer interferometry (BLI) or surface plasmon resonance (SPR) assays by Octet®, using, for example, an Octet® Red96 system, or by Biacore®, using, for example, a Biacore® TM-2000 or a Biacore® TM-3000. An “on-rate” or “rate of association” or “association rate” or “kon” may also be determined with the same biolayer interferometry (BLI) or surface plasmon resonance (SPR) techniques described above using, for example, the Octet® Red96, the Biacore® TM-2000, or the Biacore® TM-3000 system.

The terms “polypeptide” and “peptide” and “protein” are used interchangeably herein and refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification. Also included within the definition are, for example, polypeptides containing one or more analogs of an amino acid, including but not limited to, unnatural amino acids, as well as other modifications known in the art. It is understood that, because the polypeptides of this disclosure may be based upon antibodies or other members of the immunoglobulin superfamily, in certain embodiments, a “polypeptide” can occur as a single chain or as two or more associated chains.

The term “antibody” is used in its broadest sense and encompasses various antibody structures, including but not limited to monoclonal antibodies, polyclonal antibodies, multispecific antibodies (e.g., bispecific antibodies), full-length antibodies and antigen-binding fragments thereof, so long as they exhibit the desired antigen-binding activity. The term “antibody moiety” refers to a full-length antibody or an antigen-binding fragment thereof.

A full-length antibody comprises two heavy chains and two light chains. The variable regions of the light and heavy chains are responsible for antigen binding. The variable domains of the heavy chain and light chain may be referred to as “V_{H}” and “V_{L}”, respectively. The variable regions in both chains generally contain three highly variable loops called the complementarity determining regions (CDRs) (light chain (LC) CDRs including LC-CDR1, LC-CDR2, and LC-CDR3, heavy chain (HC) CDRs including HC-CDR1, HC-CDR2, and HC-CDR3). CDR boundaries for the antibodies and antigen-binding fragments disclosed herein may be defined or identified by the conventions of Kabat, Chothia, or Al-Lazikani (Al-Lazikani 1997; Chothia 1985; Chothia 1987; Chothia 1989; Kabat 1987; Kabat 1991). The three CDRs of the heavy or light chains are interposed between flanking stretches known as framework regions (FRs), which are more highly conserved than the CDRs and form a scaffold to support the hypervariable loops. The constant regions of the heavy and light chains are not involved in antigen binding, but exhibit various effector functions. Antibodies are assigned to classes based on the amino acid sequence of the constant region of their heavy chain. The five major classes or isotypes of antibodies are IgA, IgD, IgE, IgG, and IgM, which are characterized by the presence of α, δ, ε, γ, and μ heavy chains, respectively. Several of the major antibody classes are divided into subclasses such as lgG1 (γ1 heavy chain), lgG2 (γ2 heavy chain), lgG3 (γ3 heavy chain), lgG4 (γ4 heavy chain), lgA1 (α1 heavy chain), or lgA2 (α2 heavy chain).

The term “antigen-binding fragment” as used herein refers to an antibody fragment including, for example, a diabody, a Fab, a Fab′, a F(ab′)₂, an Fv fragment, a disulfide stabilized Fv fragment (dsFv), a (dsFv)₂, a bispecific dsFv (dsFv-dsFv′), a disulfide stabilized diabody (ds diabody), a single-chain Fv (scFv), an scFv dimer (bivalent diabody), a multispecific antibody formed from a portion of an antibody comprising one or more CDRs, a single domain antibody (e.g., a camelized single domain antibody), a nanobody, a domain antibody, a bivalent domain antibody, or any other antibody fragment that binds to an antigen but does not comprise a complete antibody structure. An antigen-binding fragment is capable of binding to the same antigen to which the parent antibody or a parent antibody fragment (e.g., a parent scFv) binds. In some embodiments, an antigen-binding fragment may comprise one or more CDRs from a particular human antibody grafted to a framework region from one or more different human antibodies.

“Single-chain Fv,” also abbreviated as “sFv” or “scFv,” are antibody fragments that comprise the V_{H} and V_{L} antibody domains connected into a single polypeptide chain. In some embodiments, the scFv polypeptide further comprises a polypeptide linker between the V_{H} and V_{L} domains which enables the scFv to form the desired structure for antigen binding. For a review of scFv, see Plückthun in The Pharmacology of Monoclonal Antibodies, vol. 113, Rosenburg and Moore eds., Springer-Verlag, New York, pp. 269-315 (1994).

As used herein, the term “CDR” or “complementarity determining region” is intended to mean the non-contiguous antigen combining sites found within the variable region of both heavy and light chain polypeptides. These particular regions have been described by Kabat et al., J. Biol. Chem. 252:6609-6616 (1977); Kabat et al., U.S. Dept. of Health and Human Services, “Sequences of proteins of immunological interest” (1991); Chothia et al., J. Mol. Biol. 196:901-917 (1987); Al-Lazikani B. et al., J. Mol. Biol., 273: 927-948 (1997); MacCallum et al., J. Mol. Biol. 262:732-745 (1996); Abhinandan and Martin, Mol. Immunol., 45: 3832-3839 (2008); Lefranc M. P. et al., Dev. Comp. Immunol., 27: 55-77 (2003); and Honegger and Pluckthun, J. Mol. Biol., 309:657-670 (2001), where the definitions include overlapping or subsets of amino acid residues when compared against each other. Nevertheless, application of either definition to refer to a CDR of an antibody or grafted antibodies or variants thereof is intended to be within the scope of the term as defined and used herein. The amino acid residues which encompass the CDRs as defined by each of the above cited references are set forth below in Table 1 as a comparison. CDR prediction algorithms and interfaces are known in the art, including, for example, Abhinandan and Martin, Mol. Immunol., 45: 3832-3839 (2008); Ehrenmann F. et al., Nucleic Acids Res., 38: D301-D307 (2010); and Adolf-Bryfogle J. et al., Nucleic Acids Res., 43: D432-D438 (2015). The contents of the references cited in this paragraph are incorporated herein by reference in their entireties for use in the present application and for possible inclusion in one or more claims herein.

TABLE 1
CDR DEFINITIONS

| | Kabat¹ | Chothia² | MacCallum³ | IMGT⁴ | AHo⁵ |
|---|---|---|---|---|---|
| V_{H} CDR1 | 31-35 | 26-32 | 30-35 | 27-38 | 25-40 |
| V_{H} CDR2 | 50-65 | 53-55 | 47-58 | 56-65 | 58-77 |
| V_{H} CDR3 | 95-102 | 96-101 | 93-101 | 105-117 | 109-137 |
| V_{L} CDR1 | 24-34 | 26-32 | 30-36 | 27-38 | 25-40 |
| V_{L} CDR2 | 50-56 | 50-52 | 46-55 | 56-65 | 58-77 |
| V_{L} CDR3 | 89-97 | 91-96 | 89-96 | 105-117 | 109-137 |

¹Residue numbering follows the nomenclature of Kabat et al., supra
²Residue numbering follows the nomenclature of Chothia et al., supra
³Residue numbering follows the nomenclature of MacCallum et al., supra
⁴Residue numbering follows the nomenclature of Lefranc et al., supra
⁵Residue numbering follows the nomenclature of Honegger and Plückthun, supra

The expression “variable-domain residue-numbering as in Kabat” or “amino-acid-position numbering as in Kabat,” and variations thereof, refers to the numbering system used for heavy-chain variable domains or light-chain variable domains of the compilation of antibodies in Kabat et al., supra. Using this numbering system, the actual linear amino acid sequence may contain fewer or additional amino acids corresponding to a shortening of, or insertion into, a FR or hypervariable region (HVR) of the variable domain. For example, a heavy-chain variable domain may include a single amino acid insert (residue 52a according to Kabat) after residue 52 of H2 and inserted residues (e.g. residues 82a, 82b, and 82c, etc. according to Kabat) after heavy-chain FR residue 82. The Kabat numbering of residues may be determined for a given antibody by alignment at regions of homology of the sequence of the antibody with a “standard” Kabat numbered sequence.

Unless indicated otherwise herein, the numbering of the residues in an immunoglobulin heavy chain is that of the EU index as in Kabat et al., supra with minor modification. Briefly, we added 5 more residues in super variable loop before the heavy chain CDR1. The “EU index as in Kabat” refers to the residue numbering of the human IgG1 EU antibody.

“Framework” or “FR” residues are those variable-domain residues other than the CDR residues as herein defined.

“Humanized” forms of non-human (e.g., rodent) antibodies are chimeric antibodies that contain minimal sequence derived from the non-human antibody. For the most part, humanized antibodies are human immunoglobulins (recipient antibody) in which residues from a hypervariable region (HVR) of the recipient are replaced by residues from a hypervariable region of a non-human species (donor antibody) such as mouse, rat, rabbit or non-human primate having the desired antibody specificity, affinity, and capability. In some instances, framework region (FR) residues of the human immunoglobulin are replaced by corresponding non-human residues. Furthermore, humanized antibodies can comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992).

A “human antibody” is an antibody that possesses an amino-acid sequence corresponding to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art, including phage-display libraries. Hoogenboom and Winter, J. Mol. Biol., 227:381 (1991); Marks et al., J. Mol. Biol., 222:581 (1991). Also available for the preparation of human monoclonal antibodies are methods described in Cole et al., Monoclonal Antibodies and Cancer Therapy, Alan R. Liss, p. 77 (1985); Boerner et al., J. Immunol., 147(1):86-95 (1991). See also van Dijk and van de Winkel, Curr. Opin. Pharmacol., 5: 368-74 (2001). Human antibodies can be prepared by administering the antigen to a transgenic animal that has been modified to produce such antibodies in response to antigenic challenge, but whose endogenous loci have been disabled, e.g., immunized xenomice (see, e.g., U.S. Pat. Nos. 6,075,181 and 6,150,584 regarding XENOMOUSE™ technology). See also, for example, Li et al., Proc. Natl. Acad. Sci. USA, 103:3557-3562 (2006) regarding human antibodies generated via a human B-cell hybridoma technology.

“Percent (%) amino acid sequence identity” or “homology” with respect to the polypeptide and antibody sequences identified herein is defined as the percentage of amino acid residues in a candidate sequence that are identical with the amino acid residues in the polypeptide being compared, after aligning the sequences considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, Megalign (DNASTAR), or MUSCLE software. Those skilled in the art can determine appropriate parameters for measuring alignment, including any algorithms needed to achieve maximal alignment over the full-length of the sequences being compared. For purposes herein, however, % amino acid sequence identity values are generated using the sequence comparison computer program MUSCLE (Edgar, R. C., Nucleic Acids Research 32(5):1792-1797, 2004; Edgar, R. C., BMC Bioinformatics 5(1): 113, 2004).

“Homologous” refers to the sequence similarity or sequence identity between two polypeptides or between two nucleic acid molecules. When a position in both of the two compared sequences is occupied by the same base or amino acid monomer subunit, e.g., if a position in each of two DNA molecules is occupied by adenine, then the molecules are homologous at that position. The percent of homology between two sequences is a function of the number of matching or homologous positions shared by the two sequences divided by the number of positions compared times 100. For example, if 6 of 10 of the positions in two sequences are matched or homologous then the two sequences are 60% homologous. By way of example, the DNA sequences ATTGCC and TATGGC share 50% homology. Generally, a comparison is made when two sequences are aligned to give maximum homology.

The term “constant domain” refers to the portion of an immunoglobulin molecule having a more conserved amino acid sequence relative to the other portion of the immunoglobulin, the variable domain, which contains the antigen-binding site. The constant domain contains the C_{H}1, C_{H}2 and C_{H}3 domains (collectively, C_{H}) of the heavy chain and the CHL (or C_{L}) domain of the light chain.

The “light chains” of antibodies (immunoglobulins) from any mammalian species can be assigned to one of two clearly distinct types, called kappa (“κ”) and lambda (“λ”), based on the amino acid sequences of their constant domains.

The “CH1 domain” (also referred to as “C1” of “H1” domain) usually extends from about amino acid 118 to about amino acid 215 (EU numbering system).

“Hinge region” is generally defined as a region in IgG corresponding to Glu216 to Pro230 of human IgG1 (Burton, Molec. Immunol. 22:161-206 (1985)). Hinge regions of other IgG isotypes may be aligned with the IgG1 sequence by placing the first and last cysteine residues forming inter-heavy chain S—S bonds in the same positions.

The “CH2 domain” of a human IgG Fc region (also referred to as “C2” domain) usually extends from about amino acid 231 to about amino acid 340. The CH2 domain is unique in that it is not closely paired with another domain. Rather, two N-linked branched carbohydrate chains are interposed between the two CH2 domains of an intact native IgG molecule. It has been speculated that the carbohydrate may provide a substitute for the domain-domain pairing and help stabilize the CH2 domain. Burton, MolecImmunol. 22:161-206 (1985).

The “CH₃ domain” (also referred to as “C2” domain) comprises the stretch of residues C-terminal to a CH2 domain in an Fc region (i.e. from about amino acid residue 341 to the C-terminal end of an antibody sequence, typically at amino acid residue 446 or 447 of an IgG).

The term “Fc region” or “fragment crystallizable region” herein is used to define a C-terminal region of an immunoglobulin heavy chain, including native-sequence Fc regions and variant Fc regions. Although the boundaries of the Fc region of an immunoglobulin heavy chain might vary, the human IgG heavy-chain Fc region is usually defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue. Suitable native-sequence Fc regions for use in the antibodies described herein include human IgG1, IgG2 (IgG2A, IgG2B), IgG3 and IgG4.

“Fc receptor” or “FcR” describes a receptor that binds the Fc region of an antibody. The preferred FcR is a native sequence human FcR. Moreover, a preferred FcR is one which binds an IgG antibody (a gamma receptor) and includes receptors of the FcγRT, FcγRII, and FcγRIII subclasses, including allelic variants and alternatively spliced forms of these receptors, FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain. (See M. Daëron, Annu. Rev. Immunol. 15:203-234 (1997). FcRs are reviewed in Ravetch and Kinet, Annu. Rev. Immunol. 9: 457-92 (1991); Capel et al., Immunomethods 4: 25-34 (1994); and de Haas et al., J. Lab. Clin. Med. 126: 330-41 (1995). Other FcRs, including those to be identified in the future, are encompassed by the term “FcR” herein.

The term “epitope” as used herein refers to the specific group of atoms or amino acids on an antigen to which an antibody or antibody moiety binds. Two antibodies or antibody moieties may bind the same epitope within an antigen if they exhibit competitive binding for the antigen.

As used herein, a first antibody or fragment thereof “competes” for binding to a target antigen with a second antibody or fragment thereof when the first antibody or fragment thereof inhibits the target antigen binding of the second antibody of fragment thereof by at least about 50% (such as at least about any one of 55%, 60%, 65%, 70%, 75%, 80%, 85%, 90%, 95%, 98% or 99%) in the presence of an equimolar concentration of the first antibody or fragment thereof, or vice versa. A high throughput process for “binning” antibodies based upon their cross-competition is described in PCT Publication No. WO 03/48731.

As use herein, the terms “specifically binds,” “specifically recognizing,” and “is specific for” refer to measurable and reproducible interactions, such as binding between a target and an antibody or antibody moiety, which is determinative of the presence of the target in the presence of a heterogeneous population of molecules, including biological molecules. For example, an antibody or antibody moiety that specifically recognizes a target (which can be an epitope) is an antibody or antibody moiety that binds this target with greater affinity, avidity, more readily, and/or with greater duration than its bindings to other targets. In some embodiments, the extent of binding of an antibody to an unrelated target is less than about 10% of the binding of the antibody to the target as measured, e.g., by a radioimmunoassay (RIA). In some embodiments, an antibody that specifically binds a target has a dissociation constant (K_{D}) of ≤10⁻⁵ M, ≤10⁻⁶ M, ≤10⁻⁷ M, ≤10⁻⁸ M, ≤10⁻⁹ M, ≤10⁻¹⁰ M, ≤10⁻¹¹ M, or ≤10⁻¹² M. In some embodiments, an antibody specifically binds an epitope on a protein that is conserved among the protein from different species. In some embodiments, specific binding can include, but does not require exclusive binding. Binding specificity of the antibody or antigen-binding domain can be determined experimentally by methods known in the art. Such methods comprise, but are not limited to Western blots, ELISA-, RIA-, ECL-, IRMA-, EIA-, BIACORE™-tests and peptide scans.

An “isolated” antibody (or construct) is one that has been identified, separated and/or recovered from a component of its production environment (e.g., natural or recombinant). Preferably, the isolated polypeptide is free of association with all other components from its production environment.

“Polynucleotide” or “nucleic acid,” as used interchangeably herein, refers to polymers of nucleotides of any length and includes DNA and RNA. The nucleotides can be deoxyribonucleotides, ribonucleotides, modified nucleotides or bases, and/or their analogs, or any substrate that can be incorporated into a polymer by DNA or RNA polymerase or by a synthetic reaction. A polynucleotide may comprise modified nucleotides, such as methylated nucleotides and their analogs. “Oligonucleotide,” as used herein, refers to short, generally single-stranded, synthetic polynucleotides that are generally, but not necessarily, fewer than about 200 nucleotides in length. The terms “oligonucleotide” and “polynucleotide” are not mutually exclusive. The description above for polynucleotides is equally and fully applicable to oligonucleotides.

An “isolated” nucleic acid molecule encoding a construct, antibody, or antigen-binding fragment thereof described herein is a nucleic acid molecule that is identified and separated from at least one contaminant nucleic acid molecule with which it is ordinarily associated in the environment in which it was produced. Preferably, the isolated nucleic acid is free of association with all components associated with the production environment. The isolated nucleic acid molecules encoding the polypeptides and antibodies described herein is in a form other than in the form or setting in which it is found in nature. Isolated nucleic acid molecules therefore are distinguished from nucleic acid encoding the polypeptides and antibodies described herein existing naturally in cells. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading frame. However, enhancers do not have to be contiguous. Linking is accomplished by ligation at convenient restriction sites. If such sites do not exist, the synthetic oligonucleotide adaptors or linkers are used in accordance with conventional practice.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, and may contain mutations. Mutant progeny that have the same function or biological activity as screened or selected for in the originally transformed cell are included herein.

As used herein, “treatment” or “treating” is an approach for obtaining beneficial or desired results, including clinical results. For purposes of this application, beneficial or desired clinical results include, but are not limited to, one or more of the following: alleviating one or more symptoms resulting from the disease, diminishing the extent of the disease, stabilizing the disease (e.g., preventing or delaying the worsening of the disease), preventing or delaying the spread (e.g., metastasis) of the disease, preventing or delaying the recurrence of the disease, delaying or slowing the progression of the disease, ameliorating the disease state, providing a remission (partial or total) of the disease, decreasing the dose of one or more other medications required to treat the disease, delaying the progression of the disease, increasing or improving the quality of life, increasing weight gain, and/or prolonging survival. Also encompassed by “treatment” is a reduction of pathological consequence of cancer (such as, for example, tumor volume). The methods of the application contemplate any one or more of these aspects of treatment.

In the context of cancer, the term “treating” includes any or all of: inhibiting growth of cancer cells, inhibiting replication of cancer cells, lessening of overall tumor burden and ameliorating one or more symptoms associated with the disease.

The terms “inhibition” or “inhibit” refer to a decrease or cessation of any phenotypic characteristic or to the decrease or cessation in the incidence, degree, or likelihood of that characteristic. To “reduce” or “inhibit” is to decrease, reduce or arrest an activity, function, and/or amount as compared to that of a reference. In certain embodiments, by “reduce” or “inhibit” is meant the ability to cause an overall decrease of 20% or greater. In another embodiment, by “reduce” or “inhibit” is meant the ability to cause an overall decrease of 50% or greater. In yet another embodiment, by “reduce” or “inhibit” is meant the ability to cause an overall decrease of 75%, 85%, 90%, 95%, or greater.

A “reference” as used herein, refers to any sample, standard, or level that is used for comparison purposes. A reference may be obtained from a healthy and/or non-diseased sample. In some examples, a reference may be obtained from an untreated sample. In some examples, a reference is obtained from a non-diseased or non-treated sample of an individual. In some examples, a reference is obtained from one or more healthy individuals who are not the individual or patient.

As used herein, “delaying development of a disease” means to defer, hinder, slow, retard, stabilize, suppress and/or postpone development of the disease (such as cancer). This delay can be of varying lengths of time, depending on the history of the disease and/or individual being treated. As is evident to one skilled in the art, a sufficient or significant delay can, in effect, encompass prevention, in that the individual does not develop the disease. For example, a late stage cancer, such as development of metastasis, may be delayed.

“Preventing” as used herein, includes providing prophylaxis with respect to the occurrence or recurrence of a disease in an individual that may be predisposed to the disease but has not yet been diagnosed with the disease.

As used herein, to “suppress” a function or activity is to reduce the function or activity when compared to otherwise same conditions except for a condition or parameter of interest, or alternatively, as compared to another condition. For example, an antibody which suppresses tumor growth reduces the rate of growth of the tumor compared to the rate of growth of the tumor in the absence of the antibody.

The terms “subject,” “individual,” and “patient” are used interchangeably herein to refer to a mammal, including, but not limited to, human, bovine, horse, feline, canine, rodent, or primate. In some embodiments, the individual is a human.

An “effective amount” of an agent refers to an amount effective, at dosages and for periods of time necessary, to achieve the desired therapeutic result. The specific dose may vary depending on one or more of: the particular agent chosen, the dosing regimen to be followed, whether it is administered in combination with other compounds, timing of administration, the tissue to be imaged, and the physical delivery system in which it is carried.

A “therapeutically effective amount” of a substance/molecule of the application, agonist or antagonist may vary according to factors such as the disease state, age, sex, and weight of the individual, and the ability of the substance/molecule, agonist or antagonist to elicit a desired response in the individual. A therapeutically effective amount is also one in which any toxic or detrimental effects of the substance/molecule, agonist or antagonist are outweighed by the therapeutically beneficial effects. A therapeutically effective amount may be delivered in one or more administrations.

The terms “pharmaceutical formulation” and “pharmaceutical composition” refer to a preparation which is in such form as to permit the biological activity of the active ingredient(s) to be effective, and which contains no additional components which are unacceptably toxic to an individual to which the formulation would be administered. Such formulations may be sterile.

A “pharmaceutically acceptable carrier” refers to a non-toxic solid, semisolid, or liquid filler, diluent, encapsulating material, formulation auxiliary, or carrier conventional in the art for use with a therapeutic agent that together comprise a “pharmaceutical composition” for administration to an individual. A pharmaceutically acceptable carrier is non-toxic to recipients at the dosages and concentrations employed and is compatible with other ingredients of the formulation. The pharmaceutically acceptable carrier is appropriate for the formulation employed.

A “sterile” formulation is aseptic or essentially free from living microorganisms and their spores.

Administration “in combination with” one or more further therapeutic agents includes simultaneous (concurrent) and consecutive or sequential administration in any order.

The term “concurrently” is used herein to refer to administration of two or more therapeutic agents, where at least part of the administration overlaps in time or where the administration of one therapeutic agent falls within a short period of time relative to administration of the other therapeutic agent. For example, the two or more therapeutic agents are administered with a time separation of no more than about 60 minutes, such as no more than about any of 30, 15, 10, 5, or 1 minutes.

The term “sequentially” is used herein to refer to administration of two or more therapeutic agents where the administration of one or more agent(s) continues after discontinuing the administration of one or more other agent(s). For example, administration of the two or more therapeutic agents are administered with a time separation of more than about 15 minutes, such as about any of 20, 30, 40, 50, or 60 minutes, 1 day, 2 days, 3 days, 1 week, 2 weeks, or 1 month, or longer.

As used herein, “in conjunction with” refers to administration of one treatment modality in addition to another treatment modality. As such, “in conjunction with” refers to administration of one treatment modality before, during or after administration of the other treatment modality to the individual.

The term “package insert” is used to refer to instructions customarily included in commercial packages of therapeutic products, that contain information about the indications, usage, dosage, administration, combination therapy, contraindications and/or warnings concerning the use of such therapeutic products.

An “article of manufacture” is any manufacture (e.g., a package or container) or kit comprising at least one reagent, e.g., a medicament for treatment of a disease or disorder (e.g., cancer), or a probe for specifically detecting a biomarker described herein. In certain embodiments, the manufacture or kit is promoted, distributed, or sold as a unit for performing the methods described herein.

It is understood that embodiments of the application described herein include “consisting” and/or “consisting essentially of” embodiments.

Reference to “about” a value or parameter herein includes (and describes) variations that are directed to that value or parameter per se. For example, description referring to “about X” includes description of “X”.

As used herein, reference to “not” a value or parameter generally means and describes “other than” a value or parameter. For example, the method is not used to treat cancer of type X means the method is used to treat cancer of types other than X.

The term “about X-Y” used herein has the same meaning as “about X to about Y.”

As used herein and in the appended claims, the singular forms “a,” “or,” and “the” include plural referents unless the context clearly dictates otherwise.

### II. Fusion Proteins and TGFβR Ectodomain Polypeptides

The present application provides fusion proteins that comprise one or more a TGFβ superfamily receptor ectodomain (such as any of these described herein) and a second moiety. In some embodiments, the fusion protein further comprises an anti-PD-L1 antibody moiety (such as an anti-PD-L1 full length antibody). In some embodiments, the fusion protein further comprises a half-life extending moiety.

Fusion Proteins that Comprise a TGFβ Superfamily Receptor Ectodomain and an Anti-PD-L1 Antibody Moiety

The present application in one aspect provides fusion proteins that comprise a TGFβ superfamily receptor ectodomain (such as any of the TGFβ superfamily receptor ectodomain as described herein) and an anti-PD-L1 antibody moiety (such as any of the anti-PD-L1 antibody moiety described herein).

In some embodiments, there is provided a fusion protein comprising a) a full-length antibody that specifically binds to PD-L1 and comprises two heavy chains and two light chains, wherein each of the two heavy chains comprises a heavy chain variable region (V_{H}) and each of the two light chains comprises a light chain variable region (V_{L}); b) a TGFβ superfamily receptor ectodomain; wherein the TGFβ superfamily receptor ectodomain is fused to at least one or both of the heavy chains or light chains of the anti-PD-L1 antibody. In some embodiments, full-length antibody comprises Fc fragment derived from a human IgG1. In some embodiments, the Fc fragment derived from human IgG1 comprises a K214R mutation and an A297N mutation. In some embodiments, the TGFβ superfamily receptor ectodomain comprises a TGFβ receptor type II ectodomain or a variant thereof. In some embodiments, the TGFβ receptor type II ectodomain or a variant thereof comprises a human TGFβ receptor type II ectodomain or a variant thereof. In some embodiments, the human TGFβ receptor type II ectodomain or a variant thereof comprises a variant human TGFβ receptor type II ectodomain that has at least about 90% sequence identity to SEQ ID NO: 2, wherein the variant comprises one or more amino acid substitution at position N47, N71, and/or N131 according to the numbering of SEQ ID NO: 2. In some embodiments, the variant comprises the amino acid sequence of SEQ ID NO: 3. In some embodiments, the TGFβ superfamily receptor ectodomain comprises a TGFβ receptor type I ectodomain or a variant thereof. In some embodiments, the TGFβ receptor type I ectodomain or a variant thereof comprises a human TGFβ receptor type I ectodomain or a variant thereof. In some embodiments, the human TGFβ receptor type I ectodomain or a variant thereof comprises an amino acid sequence of SEQ ID NO: 1 or a variant that has at least about 90% sequence identity. In some embodiments, the TGFβ superfamily receptor ectodomain is fused to at least one of the heavy chains or light chains of the anti-PD-L1 antibody via a linker. In some embodiments, the linker is a peptide linker. In some embodiments, the peptide linker has a length of about 2 to about 25 amino acids (such as about 2 to about 10 amino acids). In some embodiments, the linker comprises the amino acid sequence of SEQ ID NOs: 56-68.

In some embodiments, there is provided a fusion protein comprising a) a full-length antibody that specifically binds to PD-L1 and comprises two heavy chains and two light chains, wherein each of the two heavy chains comprises a heavy chain variable region (V_{H}) and each of the two light chains comprises a light chain variable region (V_{L}); b) a TGFβ superfamily receptor ectodomain; wherein the TGFβ superfamily receptor ectodomain is fused to at least one or both of the light chains of the anti-PD-L1 antibody. In some embodiments, the TGFβ superfamily receptor ectodomain is fused to the C-terminus of the at least one or both of the two light chains. In some embodiments, the TGFβ superfamily receptor ectodomain is fused to the N-terminus of the at least one or both of the two light chains. In some embodiments, the TGFβ superfamily receptor ectodomain is fused to both the N-terminus and the C-terminus of the at least one or both of the two light chains. In some embodiments, full-length antibody comprises Fc fragment derived from a human IgG1. In some embodiments, the Fc fragment derived from human IgG1 comprises a K214R mutation and an A297N mutation. In some embodiments, the TGFβ superfamily receptor ectodomain comprises a TGFβ receptor type II ectodomain or a variant thereof. In some embodiments, the TGFβ receptor type II ectodomain or a variant thereof comprises a human TGFβ receptor type II ectodomain or a variant thereof. In some embodiments, the human TGFβ receptor type II ectodomain or a variant thereof comprises a variant human TGFβ receptor type II ectodomain that has at least about 90% sequence identity to SEQ ID NO: 2, wherein the variant comprises one or more amino acid substitution at position N47, N71, and/or N131 according to the numbering of SEQ ID NO: 2. In some embodiments, the variant comprises the amino acid sequence of SEQ ID NO: 3. In some embodiments, the TGFβ superfamily receptor ectodomain comprises a TGFβ receptor type I ectodomain or a variant thereof. In some embodiments, the TGFβ receptor type I ectodomain or a variant thereof comprises a human TGFβ receptor type I ectodomain or a variant thereof. In some embodiments, the human TGFβ receptor type I ectodomain or a variant thereof comprises an amino acid sequence of SEQ ID NO: 1 or a variant that has at least about 90% sequence identity. In some embodiments, the TGFβ superfamily receptor ectodomain is fused to at least one of the heavy chains or light chains of the anti-PD-L1 antibody via a linker. In some embodiments, the linker is a peptide linker. In some embodiments, the peptide linker has a length of about 2 to about 25 amino acids (such as about 2 to about 10 amino acids). In some embodiments, the linker comprises the amino acid sequence of SEQ ID NOs: 56-68.

In some embodiments, there is provided a fusion protein comprising a) a full-length antibody that specifically binds to PD-L1 and comprises two heavy chains and two light chains, wherein each of the two heavy chains comprises a heavy chain variable region (V_{H}) and each of the two light chains comprises a light chain variable region (V_{L}); b) a TGFβ superfamily receptor ectodomain; wherein the TGFβ superfamily receptor ectodomain is fused to at least one or both of the heavy chains of the anti-PD-L1 antibody. In some embodiments, the TGFβ superfamily receptor ectodomain is fused to the C-terminus of the at least one or both of the two heavy chains. In some embodiments, the TGFβ superfamily receptor ectodomain is fused to the N-terminus of the at least one or both of the two heavy chains. In some embodiments, the TGFβ superfamily receptor ectodomain is fused to both the N-terminus and the C-terminus of the at least one or both of the two heavy chains. In some embodiments, full-length antibody comprises Fc fragment derived from a human IgG1. In some embodiments, the Fc fragment derived from human IgG1 comprises a K214R mutation and an A297N mutation. In some embodiments, the TGFβ superfamily receptor ectodomain comprises a TGFβ receptor type II ectodomain or a variant thereof. In some embodiments, the TGFβ receptor type II ectodomain or a variant thereof comprises a human TGFβ receptor type II ectodomain or a variant thereof. In some embodiments, the human TGFβ receptor type II ectodomain or a variant thereof comprises a variant human TGFβ receptor type II ectodomain that has at least about 90% sequence identity to SEQ ID NO: 2, wherein the variant comprises one or more amino acid substitution at position N47, N71, and/or N131 according to the numbering of SEQ ID NO: 2. In some embodiments, the variant comprises the amino acid sequence of SEQ ID NO: 3. In some embodiments, the TGFβ superfamily receptor ectodomain comprises a TGFβ receptor type I ectodomain or a variant thereof. In some embodiments, the TGFβ receptor type I ectodomain or a variant thereof comprises a human TGFβ receptor type I ectodomain or a variant thereof. In some embodiments, the human TGFβ receptor type I ectodomain or a variant thereof comprises an amino acid sequence of SEQ ID NO: 1 or a variant that has at least about 90% sequence identity. In some embodiments, the TGFβ superfamily receptor ectodomain is fused to at least one of the heavy chains or light chains of the anti-PD-L1 antibody via a linker. In some embodiments, the linker is a peptide linker. In some embodiments, the peptide linker has a length of about 2 to about 25 amino acids (such as about 2 to about 10 amino acids). In some embodiments, the linker comprises the amino acid sequence of SEQ ID NOs: 56-68.

In some embodiments, there is provided a fusion protein comprising a) a full-length antibody that specifically binds to PD-L1 and comprises two heavy chains and two light chains, wherein each of the two heavy chains comprises a heavy chain variable region (V_{H}) and each of the two light chains comprises a light chain variable region (V_{L}); b) a TGFβ superfamily receptor ectodomain; wherein the TGFβ superfamily receptor ectodomain is fused to i) at least one or both of the light chains of the anti-PD-L1 antibody and ii) at least one or both of the heavy chains of the anti-PD-L1 antibody. In some embodiments, the TGFβ superfamily receptor ectodomain is fused to the C-terminus of i) the two heavy chains and ii) two light chains. In some embodiments, the TGFβ superfamily receptor ectodomain is fused to the N-terminus of i) the two heavy chains and ii) two light chains. In some embodiments, the TGFβ superfamily receptor ectodomain is fused to i) the N-terminus of the two heavy chains and ii) the C-terminus of two light chains. In some embodiments, the TGFβ superfamily receptor ectodomain is fused to i) the C-terminus of the two heavy chains and ii) the N-terminus of two light chains. In some embodiments, full-length antibody comprises Fc fragment derived from a human IgG1. In some embodiments, the Fc fragment derived from human IgG1 comprises a K214R mutation and an A297N mutation. In some embodiments, the TGFβ superfamily receptor ectodomain comprises a TGFβ receptor type II ectodomain or a variant thereof. In some embodiments, the TGFβ receptor type II ectodomain or a variant thereof comprises a human TGFβ receptor type II ectodomain or a variant thereof. In some embodiments, the human TGFβ receptor type II ectodomain or a variant thereof comprises a variant human TGFβ receptor type II ectodomain that has at least about 90% sequence identity to SEQ ID NO: 2, wherein the variant comprises one or more amino acid substitution at position N47, N71, and/or N131 according to the numbering of SEQ ID NO: 2. In some embodiments, the variant comprises the amino acid sequence of SEQ ID NO: 3. In some embodiments, the TGFβ superfamily receptor ectodomain comprises a TGFβ receptor type I ectodomain or a variant thereof. In some embodiments, the TGFβ receptor type I ectodomain or a variant thereof comprises a human TGFβ receptor type I ectodomain or a variant thereof. In some embodiments, the human TGFβ receptor type I ectodomain or a variant thereof comprises an amino acid sequence of SEQ ID NO: 1 or a variant that has at least about 90% sequence identity. In some embodiments, the TGFβ superfamily receptor ectodomain is fused to at least one of the heavy chains or light chains of the anti-PD-L1 antibody via a linker. In some embodiments, the linker is a peptide linker. In some embodiments, the peptide linker has a length of about 2 to about 25 amino acids (such as about 2 to about 10 amino acids). In some embodiments, the linker comprises the amino acid sequence of SEQ ID NOs: 56-68.

In some embodiments, there is provided a fusion protein comprising a) a full-length antibody that specifically binds to PD-L1 and comprises two heavy chains and two light chains, wherein each of the two heavy chains comprises a heavy chain variable region (V_{H}) and each of the two light chains comprises a light chain variable region (V_{L}); b) a first TGFβ superfamily receptor ectodomain comprising a TGFβ receptor type I ectodomain or a variant thereof; and c) a second TGFβ superfamily receptor ectodomain comprising a TGFβ receptor type II ectodomain or a variant thereof, and wherein the first and the second TGFβ superfamily receptor ectodomains are both fused to one or both of the two heavy chains of the anti-PD-L1 antibody. In some embodiments, both of the first and the second TGFβ superfamily receptor ectodomains are fused to the N-terminus of the one or both of the two heavy chains of the anti-PD-L1 antibody. In some embodiments, both of the first and the second TGFβ superfamily receptor ectodomains are fused to the C-terminus of the one or both of the two heavy chains of the anti-PD-L1 antibody. In some embodiments, the first and the second TGFβ superfamily receptor ectodomains are linked to each other via a linker (such as any of the linkers described herein, such as a linker comprising an amino acid sequence of any of SEQ ID NOs: 56-68). In some embodiments, one of the first and the second TGFβ superfamily receptor ectodomains is fused to N-terminus of the one or both of the two heavy chains of the anti-PD-L1 antibody, and the other one of the first and the second TGFβ superfamily receptor ectodomains is fused to C-terminus of the one or both of the two heavy chains of the anti-PD-L1 antibody. In some embodiments, the TGFβ receptor type I ectodomain or a variant thereof is fused to N-terminus of the one or both of the two heavy chains of the anti-PD-L1 antibody; and the TGFβ receptor type II ectodomain or a variant thereof is fused to C-terminus of the one or both of the two heavy chains of the anti-PD-L1 antibody. In some embodiments, the TGFβ receptor type I ectodomain or a variant thereof is fused to C-terminus of the one or both of the two heavy chains of the anti-PD-L1 antibody; and the TGFβ receptor type II ectodomain or a variant thereof is fused to N-terminus of the one or both of the two heavy chains of the anti-PD-L1 antibody. In some embodiments, full-length antibody comprises Fc fragment derived from a human IgG1. In some embodiments, the Fc fragment derived from human IgG1 comprises a K214R mutation and an A297N mutation. In some embodiments, the TGFβ superfamily receptor ectodomain comprises a TGFβ receptor type II ectodomain or a variant thereof. In some embodiments, the TGFβ receptor type II ectodomain or a variant thereof comprises a human TGFβ receptor type II ectodomain or a variant thereof. In some embodiments, the human TGFβ receptor type II ectodomain or a variant thereof comprises a variant human TGFβ receptor type II ectodomain that has at least about 90% sequence identity to SEQ ID NO: 2, wherein the variant comprises one or more amino acid substitution at position N47, N71, and/or N131 according to the numbering of SEQ ID NO: 2. In some embodiments, the variant comprises the amino acid sequence of SEQ ID NO: 3. In some embodiments, the TGFβ superfamily receptor ectodomain comprises a TGFβ receptor type I ectodomain or a variant thereof. In some embodiments, the TGFβ receptor type I ectodomain or a variant thereof comprises a human TGFβ receptor type I ectodomain or a variant thereof. In some embodiments, the human TGFβ receptor type I ectodomain or a variant thereof comprises an amino acid sequence of SEQ ID NO: 1 or a variant that has at least about 90% sequence identity. In some embodiments, the TGFβ superfamily receptor ectodomain is fused to at least one of the heavy chains or light chains of the anti-PD-L1 antibody via a linker. In some embodiments, the linker is a peptide linker. In some embodiments, the peptide linker has a length of about 2 to about 25 amino acids (such as about 2 to about 10 amino acids). In some embodiments, the linker comprises the amino acid sequence of SEQ ID NOs: 56-68.

In some embodiments, there is provided a fusion protein comprising a) a full-length antibody that specifically binds to PD-L1 and comprises two heavy chains and two light chains, wherein each of the two heavy chains comprises a heavy chain variable region (V_{H}) and each of the two light chains comprises a light chain variable region (V_{L}); b) a first TGFβ superfamily receptor ectodomain comprising a TGFβ receptor type I ectodomain or a variant thereof; and c) a second TGFβ superfamily receptor ectodomain comprising a TGFβ receptor type II ectodomain or a variant thereof, and wherein the first and the second TGFβ superfamily receptor ectodomains are both fused to one or both of the two light chains of the anti-PD-L1 antibody. In some embodiments, both of the first and the second TGFβ superfamily receptor ectodomains are fused to the N-terminus of the one or both of the two light chains of the anti-PD-L1 antibody. In some embodiments, both of the first and the second TGFβ superfamily receptor ectodomains are fused to the C-terminus of the one or both of the two light chains of the anti-PD-L1 antibody. In some embodiments, the first and the second TGFβ superfamily receptor ectodomains are linked to each other via a linker (such as any of the linkers described herein, such as a linker comprising an amino acid sequence of any of SEQ ID NOs: 56-68). In some embodiments, one of the first and the second TGFβ superfamily receptor ectodomains is fused to N-terminus of the one or both of the two light chains of the anti-PD-L1 antibody, and the other one of the first and the second TGFβ superfamily receptor ectodomains is fused to C-terminus of the one or both of the two light chains of the anti-PD-L1 antibody. In some embodiments, the TGFβ receptor type I ectodomain or a variant thereof is fused to N-terminus of the one or both of the two light chains of the anti-PD-L1 antibody; and the TGFβ receptor type II ectodomain or a variant thereof is fused to C-terminus of the one or both of the two light chains of the anti-PD-L1 antibody. In some embodiments, the TGFβ receptor type I ectodomain or a variant thereof is fused to C-terminus of the one or both of the two light chains of the anti-PD-L1 antibody; and the TGFβ receptor type II ectodomain or a variant thereof is fused to N-terminus of the one or both of the two light chains of the anti-PD-L1 antibody. In some embodiments, full-length antibody comprises Fc fragment derived from a human IgG1. In some embodiments, the Fc fragment derived from human IgG1 comprises a K214R mutation and an A297N mutation. In some embodiments, the TGFβ superfamily receptor ectodomain comprises a TGFβ receptor type II ectodomain or a variant thereof. In some embodiments, the TGFβ receptor type II ectodomain or a variant thereof comprises a human TGFβ receptor type II ectodomain or a variant thereof. In some embodiments, the human TGFβ receptor type II ectodomain or a variant thereof comprises a variant human TGFβ receptor type II ectodomain that has at least about 90% sequence identity to SEQ ID NO: 2, wherein the variant comprises one or more amino acid substitution at position N47, N71, and/or N131 according to the numbering of SEQ ID NO: 2. In some embodiments, the variant comprises the amino acid sequence of SEQ ID NO: 3. In some embodiments, the TGFβ superfamily receptor ectodomain comprises a TGFβ receptor type I ectodomain or a variant thereof. In some embodiments, the TGFβ receptor type I ectodomain or a variant thereof comprises a human TGFβ receptor type I ectodomain or a variant thereof. In some embodiments, the human TGFβ receptor type I ectodomain or a variant thereof comprises an amino acid sequence of SEQ ID NO: 1 or a variant that has at least about 90% sequence identity. In some embodiments, the TGFβ superfamily receptor ectodomain is fused to at least one of the heavy chains or light chains of the anti-PD-L1 antibody via a linker. In some embodiments, the linker is a peptide linker. In some embodiments, the peptide linker has a length of about 2 to about 25 amino acids (such as about 2 to about 10 amino acids). In some embodiments, the linker comprises the amino acid sequence of SEQ ID NOs: 56-68.

In some embodiments, there is provided a fusion protein comprising a) a full-length antibody that specifically binds to PD-L1 and comprises two heavy chains and two light chains, wherein each of the two heavy chains comprises a heavy chain variable region (V_{H}) and each of the two light chains comprises a light chain variable region (V_{L}); b) a first TGFβ superfamily receptor ectodomain; and c) a second TGFβ superfamily receptor ectodomain, wherein the first TGFβ superfamily receptor ectodomain is fused to one or both of the two light chains of the anti-PD-L1 antibody, and wherein the second TGFβ superfamily receptor ectodomain is fused to one or both of the two heavy chains of the anti-PD-L1 antibody. In some embodiments, the first TGFβ superfamily receptor ectodomain is fused to N-terminus of one or both of the two light chains of the anti-PD-L1 antibody, and the second TGFβ superfamily receptor ectodomain is fused to N-terminus of one or both of the two heavy chains of the anti-PD-L1 antibody. In some embodiments, the first TGFβ superfamily receptor ectodomain is fused to C-terminus of one or both of the two light chains of the anti-PD-L1 antibody, and the second TGFβ superfamily receptor ectodomain is fused to C-terminus of one or both of the two heavy chains of the anti-PD-L1 antibody. In some embodiments, the first TGFβ superfamily receptor ectodomain is fused to N-terminus of one or both of the two light chains of the anti-PD-L1 antibody, and the second TGFβ superfamily receptor ectodomain is fused to C-terminus of one or both of the two heavy chains of the anti-PD-L1 antibody. In some embodiments, the first TGFβ superfamily receptor ectodomain is fused to C-terminus of one or both of the two light chains of the anti-PD-L1 antibody, and the second TGFβ superfamily receptor ectodomain is fused to N-terminus of one or both of the two heavy chains of the anti-PD-L1 antibody. In some embodiments, the first TGFβ receptor ectodomain comprises a TGFβ receptor type I ectodomain or a variant thereof, and the second TGFβ receptor ectodomain comprises a TGFβ receptor type II ectodomain or a variant thereof. In some embodiments, the first TGFβ receptor ectodomain comprises a TGFβ receptor type II ectodomain or a variant thereof, and the second TGFβ receptor ectodomain comprises a TGFβ receptor type I ectodomain or a variant thereof. In some embodiments, full-length antibody comprises Fc fragment derived from a human IgG1. In some embodiments, the Fc fragment derived from human IgG1 comprises a K214R mutation and an A297N mutation. In some embodiments, the TGFβ receptor type II ectodomain or a variant thereof comprises a human TGFβ receptor type II ectodomain or a variant thereof. In some embodiments, the human TGFβ receptor type II ectodomain or a variant thereof comprises a variant human TGFβ receptor type II ectodomain that has at least about 90% sequence identity to SEQ ID NO: 2, wherein the variant comprises one or more amino acid substitution at position N47, N71, and/or N131 according to the numbering of SEQ ID NO: 2. In some embodiments, the variant comprises the amino acid sequence of SEQ ID NO: 3. In some embodiments, the TGFβ superfamily receptor ectodomain comprises a TGFβ receptor type I ectodomain or a variant thereof. In some embodiments, the TGFβ receptor type I ectodomain or a variant thereof comprises a human TGFβ receptor type I ectodomain or a variant thereof. In some embodiments, the human TGFβ receptor type I ectodomain or a variant thereof comprises an amino acid sequence of SEQ ID NO: 1 or a variant that has at least about 90% sequence identity. In some embodiments, the TGFβ superfamily receptor ectodomain is fused to at least one of the heavy chains or light chains of the anti-PD-L1 antibody via a linker. In some embodiments, the linker is a peptide linker. In some embodiments, the peptide linker has a length of about 2 to about 25 amino acids (such as about 2 to about 10 amino acids). In some embodiments, the linker comprises the amino acid sequence of SEQ ID NOs: 56-68.

In some embodiments, there is provided a fusion protein comprising a) a full-length antibody that specifically binds to PD-L1 and comprises two heavy chains and two light chains, wherein each of the two heavy chains comprises a heavy chain variable region (V_{H}) and each of the two light chains comprises a light chain variable region (V_{L}); b) a TGFβ superfamily receptor ectodomain comprising a TGFβ receptor type II ectodomain or a variant thereof; wherein the TGFβ superfamily receptor ectodomain is fused to C-terminus of both of the light chains of the anti-PD-L1 antibody. In some embodiments, the fusion protein has a lower TGFβ2 binding affinity than a reference fusion protein (such as M7824). In some embodiments, the TGFβ receptor type II ectodomain or a variant thereof comprises a human TGFβ receptor type II ectodomain or a variant thereof. In some embodiments, the human TGFβ receptor type II ectodomain or a variant thereof comprises an amino acid sequence of SEQ ID NO: 2. In some embodiments, the human TGFβ receptor type II ectodomain or a variant thereof comprises a variant human TGFβ receptor type II ectodomain that has at least about 90% sequence identity to SEQ ID NO: 2, wherein the variant comprises one or more amino acid substitution at position N47, N71, and/or N131 according to the numbering of SEQ ID NO: 2. In some embodiments, the variant comprises the amino acid sequence of SEQ ID NO: 3. In some embodiments, the full-length anti-PD-L1 antibody comprises Fc fragment derived from a human IgG1. In some embodiments, the Fc fragment derived from human IgG1 comprises a K214R mutation and an A297N mutation. In some embodiments, the TGFβ superfamily receptor ectodomain is fused to the light chains of the anti-PD-L1 antibody via a linker. In some embodiments, the linker is a peptide linker. In some embodiments, the peptide linker has a length of about 2 to about 25 amino acids (such as about 2 to about 10 amino acids). In some embodiments, the linker comprises the amino acid sequence of SEQ ID NO: 56-68.

In some embodiments, there is provided a fusion protein comprising a) a full-length antibody that specifically binds to PD-L1 and comprises two heavy chains and two light chains, wherein each of the two heavy chains comprises a heavy chain variable region (V_{H}) and each of the two light chains comprises a light chain variable region (V_{L}); b) a TGFβ superfamily receptor ectodomain comprising a TGFβ receptor type I ectodomain or a variant thereof; wherein the TGFβ superfamily receptor ectodomain is fused to C-terminus of both of the light chains of the anti-PD-L1 antibody. In some embodiments, the fusion protein has a lower TGFβ2 binding affinity than a reference fusion protein (such as M7824). In some embodiments, the TGFβ receptor type I ectodomain or a variant thereof comprises a human TGFβ receptor type I ectodomain or a variant thereof. In some embodiments, the human TGFβ receptor type I ectodomain or a variant thereof comprises an amino acid sequence of SEQ ID NO: 1. In some embodiments, the full-length anti-PD-L1 antibody comprises Fc fragment derived from a human IgG1. In some embodiments, the Fc fragment derived from human IgG1 comprises a K214R mutation and an A297N mutation. In some embodiments, the TGFβ superfamily receptor ectodomain is fused to the light chains of the anti-PD-L1 antibody via a linker. In some embodiments, the linker is a peptide linker. In some embodiments, the peptide linker has a length of about 2 to about 25 amino acids (such as about 2 to about 10 amino acids). In some embodiments, the linker comprises the amino acid sequence of SEQ ID NOs: 56-68.

In some embodiments, there is provided a fusion protein comprising a) a full-length antibody that specifically binds to PD-L1 and comprises two heavy chains and two light chains, wherein each of the two heavy chains comprises a heavy chain variable region (V_{H}) and each of the two light chains comprises a light chain variable region (V_{L}); b) a TGFβ superfamily receptor ectodomain comprising the amino acid sequence of any one of SEQ ID NOs: 1-3; wherein the TGFβ superfamily receptor ectodomain is fused to C-terminus of both of the light chains of the anti-PD-L1 antibody. In some embodiments, the full-length anti-PD-L1 antibody comprises Fc fragment derived from a human IgG1. In some embodiments, the Fc fragment derived from human IgG1 comprises a K214R mutation and an A297N mutation. In some embodiments, the TGFβ superfamily receptor ectodomain is fused to the light chains of the anti-PD-L1 antibody via a linker. In some embodiments, the linker is a peptide linker. In some embodiments, the peptide linker has a length of about 2 to about 25 amino acids (such as about 2 to about 10 amino acids). In some embodiments, the linker comprises the amino acid sequence of SEQ ID NOs: 56-68.

In some embodiments, there is provided a fusion protein comprising a) a full-length antibody that specifically binds to PD-L1 and comprises two heavy chains and two light chains, wherein each of the two heavy chains comprises a heavy chain variable region (V_{H}) and each of the two light chains comprises a light chain variable region (V_{L}); b) a TGFβ superfamily receptor ectodomain comprising a TGFβ receptor type II ectodomain or a variant thereof; wherein the TGFβ superfamily receptor ectodomain is fused to C-terminus of both of the heavy chains of the anti-PD-L1 antibody. In some embodiments, the TGFβ receptor type II ectodomain or a variant thereof comprises a human TGFβ receptor type II ectodomain or a variant thereof. In some embodiments, the human TGFβ receptor type II ectodomain or a variant thereof comprises an amino acid sequence of SEQ ID NO: 2. In some embodiments, the human TGFβ receptor type II ectodomain or a variant thereof comprises a variant human TGFβ receptor type II ectodomain that has at least about 90% sequence identity to SEQ ID NO: 2, wherein the variant comprises one or more amino acid substitution at position N47, N71, and/or N131 according to the numbering of SEQ ID NO: 2. In some embodiments, the variant comprises the amino acid sequence of SEQ ID NO: 3. In some embodiments, the full-length anti-PD-L1 antibody comprises Fc fragment derived from a human IgG1. In some embodiments, the Fc fragment derived from human IgG1 comprises a K214R mutation and an A297N mutation. In some embodiments, the TGFβ superfamily receptor ectodomain is fused to the heavy chains of the anti-PD-L1 antibody via a linker. In some embodiments, the linker is a peptide linker. In some embodiments, the peptide linker has a length of about 2 to about 25 amino acids (such as about 2 to about 10 amino acids). In some embodiments, the linker comprises the amino acid sequence of SEQ ID NOs: 56-68.

In some embodiments, there is provided a fusion protein comprising a) a full-length antibody that specifically binds to PD-L1 and comprises two heavy chains and two light chains, wherein each of the two heavy chains comprises a heavy chain variable region (V_{H}) and each of the two light chains comprises a light chain variable region (V_{L}); b) a TGFβ superfamily receptor ectodomain comprising a TGFβ receptor type I ectodomain or a variant thereof; wherein the TGFβ superfamily receptor ectodomain is fused to C-terminus of both of the heavy chains of the anti-PD-L1 antibody. In some embodiments, the TGFβ receptor type I ectodomain or a variant thereof comprises a human TGFβ receptor type I ectodomain or a variant thereof. In some embodiments, the human TGFβ receptor type I ectodomain or a variant thereof comprises an amino acid sequence of SEQ ID NO: 1. In some embodiments, the full-length anti-PD-L1 antibody comprises Fc fragment derived from a human IgG1. In some embodiments, the Fc fragment derived from human IgG1 comprises a K214R mutation and an A297N mutation. In some embodiments, the TGFβ superfamily receptor ectodomain is fused to the heavy chains of the anti-PD-L1 antibody via a linker. In some embodiments, the linker is a peptide linker. In some embodiments, the peptide linker has a length of about 2 to about 25 amino acids (such as about 2 to about 10 amino acids). In some embodiments, the linker comprises the amino acid sequence of SEQ ID NOs: 56-68.

In some embodiments, there is provided a fusion protein comprising a) a full-length antibody that specifically binds to PD-L1 and comprises two heavy chains and two light chains, wherein each of the two heavy chains comprises a heavy chain variable region (V_{H}) and each of the two light chains comprises a light chain variable region (V_{L}); b) a TGFβ superfamily receptor ectodomain comprising the amino acid sequence of any one of SEQ ID NOs: 1-3; wherein the TGFβ superfamily receptor ectodomain is fused to C-terminus of both of the heavy chains of the anti-PD-L1 antibody. In some embodiments, the full-length anti-PD-L1 antibody comprises Fc fragment derived from a human IgG1. In some embodiments, the Fc fragment derived from human IgG1 comprises a K214R mutation and an A297N mutation. In some embodiments, the TGFβ superfamily receptor ectodomain is fused to the heavy chains of the anti-PD-L1 antibody via a linker. In some embodiments, the linker is a peptide linker. In some embodiments, the peptide linker has a length of about 2 to about 25 amino acids (such as about 2 to about 10 amino acids). In some embodiments, the linker comprises the amino acid sequence of SEQ ID NO: 56-68.

In some embodiments, there is provided a fusion protein comprising a) a full-length antibody that specifically binds to PD-L1 and comprises two heavy chains and two light chains, wherein each of the two heavy chains comprises a heavy chain variable region (V_{H}) and each of the two light chains comprises a light chain variable region (V_{L}); b) a first TGFβ superfamily receptor ectodomain comprising a TGFβ receptor type I ectodomain; and c) a second TGFβ superfamily receptor ectodomain comprising a TGFβ receptor type II ectodomain, wherein the first TGFβ superfamily receptor ectodomain is fused to C-terminus of the two light chains of the anti-PD-L1 antibody, optionally via a first linker, and wherein the second TGFβ superfamily receptor ectodomain is fused to C-terminus of the first TGFβ superfamily receptor ectodomain, optionally via a second linker. In some embodiments, there is provided a fusion protein comprising a) a full-length antibody that specifically binds to PD-L1 and comprises two heavy chains and two light chains, wherein each of the two heavy chains comprises a heavy chain variable region (V_{H}) and each of the two light chains comprises a light chain variable region (V_{L}); b) a first TGFβ superfamily receptor ectodomain comprising a TGFβ receptor type II ectodomain; and c) a second TGFβ superfamily receptor ectodomain comprising a TGFβ receptor type I ectodomain, wherein the first TGFβ superfamily receptor ectodomain is fused to C-terminus of the two light chains of the anti-PD-L1 antibody, optionally via a first linker, and wherein the second TGFβ superfamily receptor ectodomain is fused to C-terminus of the first TGFβ superfamily receptor ectodomain, optionally via a second linker. In some embodiments, the fusion protein has a lower TGFβ2 binding affinity than a reference fusion protein (such as M7824). In some embodiments, the TGFβ receptor type II ectodomain or a variant thereof comprises a human TGFβ receptor type II ectodomain or a variant thereof. In some embodiments, the human TGFβ receptor type II ectodomain or a variant thereof comprises the amino acid sequence of SEQ ID NO: 2 or a variant having at least about 90% sequence identity to SEQ ID NO: 2, wherein the variant comprises one or more amino acid substitution at position N47, N71, and/or N131 according to the numbering of SEQ ID NO: 2. In some embodiments, the variant comprises the amino acid sequence of SEQ ID NO: 3. In some embodiments, the TGFβ superfamily receptor ectodomain comprises a TGFβ receptor type I ectodomain or a variant thereof. In some embodiments, the TGFβ receptor type I ectodomain or a variant thereof comprises a human TGFβ receptor type I ectodomain or a variant thereof. In some embodiments, the human TGFβ receptor type I ectodomain or a variant thereof comprises an amino acid sequence of SEQ ID NO: 1 or a variant that has at least about 90% sequence identity. In some embodiments, the first and/or second linker is a peptide linker. In some embodiments, the peptide linker has a length of about 2 to about 25 amino acids (such as about 2 to about 10 amino acids). In some embodiments, the linker comprises the amino acid sequence of SEQ ID NOs: 56-68.

In some embodiments, there is provided a fusion protein comprising a) a full-length antibody that specifically binds to PD-L1 and comprises two heavy chains and two light chains, wherein each of the two heavy chains comprises a heavy chain variable region (V_{H}) and each of the two light chains comprises a light chain variable region (V_{L}); b) a first TGFβ superfamily receptor ectodomain comprising a TGFβ receptor type I ectodomain; and c) a second TGFβ superfamily receptor ectodomain comprising a TGFβ receptor type II ectodomain, wherein the first TGFβ superfamily receptor ectodomain is fused to C-terminus of the two heavy chains of the anti-PD-L1 antibody, optionally via a first linker, and wherein the second TGFβ superfamily receptor ectodomain is fused to C-terminus of the first TGFβ superfamily receptor ectodomain, optionally via a second linker. In some embodiments, there is provided a fusion protein comprising a) a full-length antibody that specifically binds to PD-L1 and comprises two heavy chains and two light chains, wherein each of the two heavy chains comprises a heavy chain variable region (V_{H}) and each of the two light chains comprises a light chain variable region (V_{L}); b) a first TGFβ superfamily receptor ectodomain comprising a TGFβ receptor type II ectodomain; and c) a second TGFβ superfamily receptor ectodomain comprising a TGFβ receptor type I ectodomain, wherein the first TGFβ superfamily receptor ectodomain is fused to C-terminus of the two heavy chains of the anti-PD-L1 antibody, optionally via a first linker, and wherein the second TGFβ superfamily receptor ectodomain is fused to C-terminus of the first TGFβ superfamily receptor ectodomain, optionally via a second linker. In some embodiments, the fusion protein has a lower TGFβ2 binding affinity than a reference fusion protein (such as M7824). In some embodiments, the TGFβ receptor type II ectodomain or a variant thereof comprises a human TGFβ receptor type II ectodomain or a variant thereof. In some embodiments, the human TGFβ receptor type II ectodomain or a variant thereof comprises the amino acid sequence of SEQ ID NO: 2 or a variant having at least about 90% sequence identity to SEQ ID NO: 2, wherein the variant comprises one or more amino acid substitution at position N47, N71, and/or N131 according to the numbering of SEQ ID NO: 2. In some embodiments, the variant comprises the amino acid sequence of SEQ ID NO: 3. In some embodiments, the TGFβ superfamily receptor ectodomain comprises a TGFβ receptor type I ectodomain or a variant thereof. In some embodiments, the TGFβ receptor type I ectodomain or a variant thereof comprises a human TGFβ receptor type I ectodomain or a variant thereof. In some embodiments, the human TGFβ receptor type I ectodomain or a variant thereof comprises an amino acid sequence of SEQ ID NO: 1 or a variant that has at least about 90% sequence identity. In some embodiments, the first and/or second linker is a peptide linker. In some embodiments, the peptide linker has a length of about 2 to about 25 amino acids (such as about 2 to about 10 amino acids). In some embodiments, the linker comprises the amino acid sequence of SEQ ID NOs: 56-68.

In some embodiments, there is provided a fusion protein comprising a) a full-length antibody that specifically binds to PD-L1 and comprises two heavy chains and two light chains, wherein each of the two heavy chains comprises a heavy chain variable region (V_{H}) and each of the two light chains comprises a light chain variable region (V_{L}); b) a first TGFβ superfamily receptor ectodomain comprising a TGFβ receptor type I ectodomain; and c) a second TGFβ superfamily receptor ectodomain comprising a TGFβ receptor type II ectodomain, wherein the first TGFβ superfamily receptor ectodomain is fused to C-terminus of the two heavy chains of the anti-PD-L1 antibody, optionally via a first linker, and wherein the second TGFβ superfamily receptor ectodomain is fused to C-terminus of the two light chains of the anti-PD-L1 antibody, optionally via a second linker. In some embodiments, there is provided a fusion protein comprising a) a full-length antibody that specifically binds to PD-L1 and comprises two heavy chains and two light chains, wherein each of the two heavy chains comprises a heavy chain variable region (V_{H}) and each of the two light chains comprises a light chain variable region (V_{L}); b) a first TGFβ superfamily receptor ectodomain comprising a TGFβ receptor type II ectodomain; and c) a second TGFβ superfamily receptor ectodomain comprising a TGFβ receptor type I ectodomain, wherein the first TGFβ superfamily receptor ectodomain is fused to C-terminus of the two heavy chains of the anti-PD-L1 antibody, optionally via a first linker, and wherein the second TGFβ superfamily receptor ectodomain is fused to C-terminus of the two light chains of the anti-PD-L1 antibody, optionally via a second linker. In some embodiments, the fusion protein has a lower TGFβ2 binding affinity than a reference fusion protein (such as M7824). In some embodiments, the TGFβ receptor type II ectodomain or a variant thereof comprises a human TGFβ receptor type II ectodomain or a variant thereof. In some embodiments, the human TGFβ receptor type II ectodomain or a variant thereof comprises the amino acid sequence of SEQ ID NO: 2 or a variant having at least about 90% sequence identity to SEQ ID NO: 2, wherein the variant comprises one or more amino acid substitution at position N47, N71, and/or N131 according to the numbering of SEQ ID NO: 2. In some embodiments, the variant comprises the amino acid sequence of SEQ ID NO: 3. In some embodiments, the TGFβ superfamily receptor ectodomain comprises a TGFβ receptor type I ectodomain or a variant thereof. In some embodiments, the TGFβ receptor type I ectodomain or a variant thereof comprises a human TGFβ receptor type I ectodomain or a variant thereof. In some embodiments, the human TGFβ receptor type I ectodomain or a variant thereof comprises an amino acid sequence of SEQ ID NO: 1 or a variant that has at least about 90% sequence identity. In some embodiments, the first and/or second linker is a peptide linker. In some embodiments, the peptide linker has a length of about 2 to about 25 amino acids (such as about 2 to about 10 amino acids). In some embodiments, the linker comprises the amino acid sequence of SEQ ID NOs: 56-68.

In some embodiments, there is provided a fusion protein comprising a) a full-length antibody that specifically binds to PD-L1 and comprises two heavy chains and two light chains, wherein each of the two heavy chains comprises a heavy chain variable region (V_{H}) and each of the two light chains comprises a light chain variable region (V_{L}); b) a TGFβ superfamily receptor ectodomain comprising the amino acid sequence of any one of SEQ ID NOs: 1-3 or a variant having at least about 90% sequence identity, wherein the TGFβ superfamily receptor ectodomain is fused to both the C-terminus of both of the heavy chains, and the C-terminus of both of the light chains of the anti-PD-L1 antibody.

In some embodiments, the fusion protein has a lower TGFβ2 binding affinity than a reference fusion protein (such as M7824, AVID200, or SHR-1701). In some embodiments, the K_{D} of the binding between the fusion protein and TGFβ2 is at least about 50%, 60%, 70%, 80%, 90%, 1-fold, 1.5-fold, 2-fold, 2.5-fold, 3-fold, 3.5-fold, 4-fold, 4.5-fold, 5-fold, 6-fold, 7-fold, 8-fold, 9-fold, or 10-fold more than the K_{D} of the binding between the reference fusion protein (such as M7824) and TGFβ2. In some embodiments, the EC50 of the fusion protein for binding to TGFβ2 is at least about 50%, 60%, 70%, 80%, 90%, 1-fold, 1.5-fold, 2-fold, 2.5-fold, 3-fold, 3.5-fold, 4-fold, 4.5-fold, 5-fold, 6-fold, 7-fold, 8-fold, 9-fold, or 10-fold more than the EC50 of reference fusion protein (such as M7824) for binding to TGFβ2.

In some embodiments, the fusion protein has a comparable TGFβ1 binding affinity as a reference fusion protein (such as M7824, AVID200, or SHR-1701). For example, in some embodiments, the K_{D} of the binding between the fusion protein and TGFβ1 is within about 50% to about 200% (such as about 50% to about 150%, about 75% to about 125%) of the K_{D} of the binding between the reference fusion protein (such as M7824) and TGFβ1. In some embodiments, the EC50 of the binding between the fusion protein and TGFβ1 is within about 50% to about 200% (such as about 50% to about 150%, about 75% to about 125%) of the EC50 of the binding between the reference fusion protein (such as M7824) and TGFβ1.

In some embodiments, the fusion protein has a comparable TGFβ3 binding affinity as a reference fusion protein (such as M7824, AVID200, or SHR-1701). For example, in some embodiments, the K_{D} of the binding between the fusion protein and TGFβ3 is within about 50% to about 200% (such as about 50% to about 150%, about 75% to about 125%) of the K_{D} of the binding between the reference fusion protein (such as M7824) and TGFβ3. In some embodiments, the EC50 of the binding between the fusion protein and TGFβ3 is within about 50% to about 200% (such as about 50% to about 150%, about 75% to about 125%) of the EC50 of the binding between the reference fusion protein (such as M7824) and TGFβ3.

In some embodiments, the fusion protein has a comparable TGFβ1 and/or TGFβ3 binding affinity as a reference fusion protein (such as M7824) while has a lower TGFβ2 binding affinity than the reference fusion protein.

### TGFβ Superfamily Receptor Ectodomain

Transforming growth factor beta (TGF-β, or TGF-beta) is a multifunctional cytokine consisting of three distinct mammalian isoforms including TGF-β1, TGF-β2 and TGF-β3. TGF-β proteins are produced by many cell types. It is expressed as a latent molecule including the latency-associated peptide (LAP) and transforming growth factor beta (TGF-β). Once activated following release of LAP by plasmin-mediated cleavage, the active TGF-β binds to TGF-β receptor (TGFβR) including TGFβR1 and TGFβR2, triggering the translocation of SMAD proteins from cytoplasm to nucleus followed by specific mRNAs generation and relative proteins expression to regulate the growth and differentiation of various cell types. Therefore, TGF-β signaling plays pivotal roles in various processes including normal mammary development and immune response.

TGF-β ligands bind to three isoforms of the TGF-β receptor (TGFβR1, TGFβR2 and TGFβR3) with different affinities. As TGF-βR1 and TGF-βR3 bind TGFβR2 with higher affinity than TGFβR1, TGF-β and TGFβR2 forms complex first and TGFβR1 is then recruited to the complex that includes one dimeric TGF-β molecule, two TGFβR1 and two TGFβR3 molecules forming a symmetric 2:2:2 complex. In addition, three TGF-β isoforms shows differential binding affinities for the TGF-β type II receptor (TGFβR2). In most cells, TGF-β1 and TGF-β3 bind to TGFβR2 with much higher affinity than TGF-β2. High levels of TGF-β1 and TGF-β3 were found in many cancers like breast, gastric and lung cancer, and blocking their interaction with TGF-β receptor will suppress tumor growth and metastasis. In contrast, TGF-β2 plays physiological roles in normal cardiac function and early hematopoiesis, and blocking of TGF-32 will lead to cardiac toxicity that has been confirmed by pan-TGF-β pathway inhibitor of small molecules in clinical trials. Thus, the ideal TGF-β blocker should selectively block the TGF-β1 and TGF-β3 signaling pathway minimizing TGF-β2 neutralization.

TGFβ inhibits local anti-tumor immunity in the tumor microenvironment that limits the effects of immune checkpoint inhibition. To overcome the resistance of immune checkpoint inhibitors, bifunctional constructs are designed herein by linking TGFβ receptor to anti-PD-L1 mAb that could improve immunotherapy efficiency. More importantly, the antibody fusion proteins are optimized to minimize the side-effect from TGF-β2 neutralization.

Single-chain recombinant traps against TGF-β superfamily growth-factors were designed from the extracellular portion of their cognate natural receptors. The extracellular segment of all these TGF-β superfamily receptors contain a single structured domain that belongs to the snake-toxin family according to SCOP (Andreeva et al., 2008, Nucl. Acid Res. 36: D419) and Pfam (Finn et al., 2006, Nucl Acid Res. 34: D247) structural classifications. The complete extracellular portion of these receptors typically includes unstructured segments flanking their folded ligand-binding domain. These unstructured extracellular portions were apparent from the experimentally determined 3D structures available from the PDB database (Berman et al., 2000, Nucl. Acid Res. 28: 235), e.g., crystal structures for type II TGF-β receptor ectodomain (Hart et al., 2002 Nat. Struct. Biol. 9: 203; Boesen et al., 2002, Structure 10: 913; Groppe et al., 2008, Mol. Cell 29: 157), type I TGF-β receptor ectodomain (Groppe et al., 2008, Mol. Cell 29:157), type IIaactivin receptor ectodomain (Allendorph et al., 2006, Proc. Natl. Acad. Sci. USA 103: 7643), type IIbactivin receptor ectodomain (Thompson et al., 2003, EMBO J. 22: 1555; Greenwald et al., 2004, Mol. Cell 15: 485), type I BMP receptor ectodomain (Kirsch et al., 2000, Nat. Struct. Biol. 7: 492), or the NMR structure of the type II TGF-β receptor ectodomain (Deep et al., 2003, Biochemistry 42: 10126)].

A. TGFβ Superfamily Receptor Type I Ectodomain

In some embodiments, the TGFβ superfamily receptor ectodomain comprises a TGFβ superfamily receptor type I ectodomain or a variant thereof.

In some embodiments, the TGFβ receptor type I ectodomain or a variant thereof comprises a human TGFβ receptor type I ectodomain or a variant thereof. In some embodiments, the human TGFβ receptor type I ectodomain or a variant thereof comprises a variant human TGFβ receptor type I ectodomain that has at least about 80% (such as at least about any of 80%, 85%, 90%, 95%, 96%, 97%, 98%, or 99%) sequence identity to SEQ ID NO: 1.

In some embodiments, the TGFβ receptor type I ectodomain or a variant thereof comprises a truncated form of the TGFβ receptor type I ectodomain. Exemplary TGFβ receptor type I ectodomains include those described in, for example, WO2017037634, which is incorporated by reference in its entirety. In some embodiments, the truncated form comprises the amino acid sequence of SEQ ID NO: 89 or 90 or a variant thereof.

B. TGFβ Superfamily Receptor Type II Ectodomain

In some embodiments, the TGFβ superfamily receptor ectodomain comprises a TGFβ superfamily receptor type II ectodomain or a variant thereof.

In some embodiments, the TGFβ receptor type II ectodomain or a variant thereof comprises a human TGFβ receptor type II ectodomain or a variant thereof. In some embodiments, the human TGFβ receptor type II ectodomain comprises a variant human TGFβ receptor type II ectodomain that has at least about 80% (such as at least about any of 80%, 85%, 90%, 95%, 96%, 97%, 98%, or 99%) sequence identity to SEQ ID NO: 2, wherein the variant comprises one or more amino acid substitution at position N47, N71, and/or N131 according to the numbering of SEQ ID NO: 2. In some embodiments, the variant comprises a N47Q mutation according to the numbering of SEQ ID NO: 2. In some embodiments, the variant comprises a N71Q mutation according to the numbering of SEQ ID NO: 2. In some embodiments, the variant comprises a N131Q mutation according to the numbering of SEQ ID NO: 2. In some embodiments, the variant comprises the amino acid sequence of SEQ ID NO: 3.

In some embodiments, the TGFβ receptor type II ectodomain or a variant thereof comprises a truncated form of the TGFβ receptor type II ectodomain (e.g., SEQ ID NO: 2 or 3). Exemplary TGFβ receptor type II ectodomains (such as truncated forms) are described in, for example, WO2018205985 and WO2017037634, which are incorporated by reference in their entirety. In some embodiments, the truncated form lacks the first 1-26 (such as first 14-21) amino acids at the N-terminus of the TGFβ receptor type II ectodomain (e.g., SEQ ID NO: 2 or 3). In some embodiments, the truncated form lacks the first 14 amino acids at the N-terminus of the TGFβ receptor type II ectodomain (e.g., SEQ ID NO: 2 or 3). In some embodiments, the truncated form lacks the first 19 amino acids at the N-terminus of the TGFβ receptor type II ectodomain (e.g., SEQ ID NO: 2 or 3). In some embodiments, the truncated form lacks the first 21 amino acids at the N-terminus of the TGFβ receptor type II ectodomain (e.g., SEQ ID NO: 2 or 3). In some embodiments, the truncated form lacks the first 26 amino acids at the N-terminus of the TGFβ receptor type II ectodomain (e.g., SEQ ID NO: 2 or 3). In some embodiments, the truncated form comprises the amino acid sequence of any one of SEQ ID NOs: 91-93 or a variant thereof.

### TGFβR Ectodomain Polypeptides and Fusion Proteins Comprising Such Polypeptides

The present application also provides non-naturally occurring polypeptides comprising a variant TGFβ receptor type II ectodomain. In some embodiments, the polypeptide comprises a variant human TGFβ receptor type II ectodomain that has at least about 80% (such as at least about any of 80%, 85%, 90%, 95%, 96%, 97%, 98%, or 99%) sequence identity to SEQ ID NO: 2, wherein the variant comprises one or more amino acid substitution at position N47, N71, and/or N131 according to the numbering of SEQ ID NO: 2. In some embodiments, the variant comprises a N47Q mutation according to SEQ ID NO: 2. In some embodiments, the variant comprises a N71Q mutation according to SEQ ID NO: 2. In some embodiments, the variant comprises a N131Q mutation according to SEQ ID NO: 2.

In some embodiments, the variant comprises the amino acid sequence of SEQ ID NO: 3.

In some embodiments, variant TGFβ receptor type II ectodomain comprises a truncated form of the TGFβ receptor type II ectodomain. In some embodiments, the truncated form comprises the amino acid sequence of SEQ ID NO: 91 or a variant thereof. In some embodiments, the truncated form comprises the amino acid sequence of SEQ ID NO: 92. In some embodiments, the truncated form comprises the amino acid sequence of SEQ ID NO: 93.

The present application also provides fusion proteins that comprise any of the polypeptides described above. In some embodiments, the fusion protein further comprises a second moiety. In some embodiments, the second moiety comprises a half-life extending domain.

In some embodiments, there if provided a fusion protein comprising a) a TGFβ superfamily receptor ectodomain comprising a variant human TGFβ receptor type II ectodomain that has at least about 80% (such as at least about any of 80%, 85%, 90%, 95%, 96%, 97%, 98%, or 99%) sequence identity to SEQ ID NO: 2, wherein the variant comprises one or more amino acid substitution at position N47, N71, and/or N131 according to the numbering of SEQ ID NO: 2 and b) a second moiety (such as a half-life extending moiety). In some embodiments, the second moiety is fused to the C-terminus of the TGFβ superfamily receptor ectodomain. In some embodiments, the second moiety is fused to the N-terminus of the TGFβ superfamily receptor ectodomain. In some embodiments, the second moiety is fused to the TGFβ superfamily receptor ectodomain via a linker (such as any of the linkers described herein).

In some embodiments, there if provided a fusion protein comprising a) a truncated form of human TGFβ receptor type II ectodomain that comprises the amino acid sequence of any one of SEQ ID NOs: 91-93, or a variant that has at least about 80% (such as at least about any of 80%, 85%, 90%, 95%, 96%, 97%, 98%, or 99%) sequence identity; and b) a second moiety (such as a half-life extending moiety). In some embodiments, the second moiety is fused to the C-terminus of the TGFβ superfamily receptor ectodomain. In some embodiments, the second moiety is fused to the N-terminus of the TGFβ superfamily receptor ectodomain. In some embodiments, the second moiety is fused to the TGFβ superfamily receptor ectodomain via a linker (such as any of the linkers described herein).

### Half-Life Extending Moiety

In some embodiments, the half-life extending moiety is an Fc fragment. In some embodiments, the half-life extending moiety is an albumin binding moiety (e.g., an albumin binding antibody moiety).

In some embodiments, the half-life extending moiety is an Fc fragment (such as any of the Fc fragments or variants thereof described herein). The term “Fc region,” “Fc domain” or “Fc” refers to a C-terminal non-antigen binding region of an immunoglobulin heavy chain that contains at least a portion of the constant region. The term includes native Fc regions and variant Fc regions. In some embodiments, a human IgG heavy chain Fc region extends from Cys226 to the carboxyl-terminus of the heavy chain. However, the C-terminal lysine (Lys447) of the Fc region may or may not be present, without affecting the structure or stability of the Fc region. Unless otherwise specified herein, numbering of amino acid residues in the IgG or Fc region is according to the EU numbering system for antibodies, also called the EU index, as described in Kabat et al., Sequences of Proteins of Immunological Interest, 5th Ed. Public Health Service, National Institutes of Health, Bethesda, Md., 1991.

In some embodiments, the Fc fragment is from an immunoglobulin selected from the group consisting of IgG, IgA, IgD, IgE, IgM, and combinations and hybrids thereof. In some embodiments, the Fc fragment is from an immunoglobulin selected from the group consisting of IgG1, IgG2, IgG3, IgG4, and combinations and hybrids thereof.

In some embodiments, the Fc fragment has a reduced effector function as compared to corresponding wildtype Fc fragment (such as at least about 30%, 40%, 50%, 60%, 70%, 80%, 85%, 90%, or 95% reduced effector function as measured by the level of antibody-dependent cellular cytotoxicity (ADCC)).

In some embodiments, the Fc fragment is an IgG1 Fc fragment. In some embodiments, the IgG1 Fc fragment comprises a L234A mutation and/or a L235Amutation. In some embodiments, the Fc fragment is an IgG2 or IgG4 Fc fragment. In some embodiments, the Fc fragment is an IgG4 Fc fragment comprising a S228P, F234A, and/or a L235A mutation. In some embodiments, the Fc fragment comprises a N297A mutation. In some embodiments, the Fc fragment comprises a N297G mutation.

In some embodiments, the TGFβ superfamily receptor ectodomain and the half-life extending moiety is linked via a linker (such as any of the linkers described in the “Linkers” section).

### Anti-PD-L1 Antibody Moiety

The anti-PD-L1 antibody moiety (such as a full-length antibody) described in the present application include any antibody moiety that specifically bind to PD-L1, for example, those described in WO2019129211, which is incorporated by reference in its entirety.

In some embodiments, the anti-PD-L1 antibody moiety comprises a full-length antibody comprising two heavy chains and two light chains. In some embodiments, the full-length antibody has an Fc fragment selected from the group consisting of Fc fragments from IgG, IgA, IgD, IgE, IgM, and combinations and hybrids thereof. In some embodiments, the Fc fragment is selected from the group consisting of Fc fragments from IgG1, IgG2, IgG3, IgG4, and combinations and hybrids thereof. In some embodiments, the Fc fragment is an IgG1 or IgG4 Fc fragment.

In some embodiments, the PD-L1 is a human PD-L1.

In some embodiments, the anti-PD-L1 antibody comprises a full-length antibody that competes for binding to PD-L1 with an antibody moiety comprising a heavy chain variable region (V_{H}) and a light chain variable region (V_{L}); wherein a) the V_{H} comprises: i) a HC-CDR1 comprising an amino acid sequence of SEQ ID NO: 6; ii) a HC-CDR2 comprising an amino acid sequence of SEQ ID NO: 7; and iii) a HC-CDR3 comprising an amino acid sequence of SEQ ID NO: 8; and, b) the V_{L} comprises: i) a LC-CDR1 comprising an amino acid sequence of SEQ ID NO: 9; ii) a LC-CDR2 comprising an amino acid sequence of SEQ ID NO: 10; and iii) a LC-CDR3 comprising an amino acid sequence of SEQ ID NO: 11.

In some embodiments, the anti-PD-L1 antibody comprises a full-length antibody comprising a heavy chain variable region (V_{H}) and a light chain variable region (V_{L}); wherein a) the V_{H} comprises: i) a HC-CDR1 comprising an amino acid sequence of SEQ ID NO: 6, or a variant thereof comprising up to about 3 (such as any of about 1, 2, 3) amino acid substitutions; ii) a HC-CDR2 comprising an amino acid sequence of SEQ ID NO: 7, or a variant thereof comprising up to about 3 (such as any of about 1, 2, 3) amino acid substitutions; and iii) a HC-CDR3 comprising an amino acid sequence of SEQ ID NO: 8, or a variant thereof comprising up to about 3 (such as any of about 1, 2, 3) amino acid substitutions; and, b) the V_{L} comprises: i) a LC-CDR1 comprising an amino acid sequence of SEQ ID NO: 9, or a variant thereof comprising up to about 3 (such as any of about 1, 2, 3) amino acid substitutions; ii) a LC-CDR2 comprising an amino acid sequence of SEQ ID NO: 10, or a variant thereof comprising up to about 3 (such as any of about 1, 2, 3) amino acid substitutions; and iii) a LC-CDR3 comprising an amino acid sequence of SEQ ID NO: 11, or a variant thereof comprising up to about 3 (such as any of about 1, 2, 3) amino acid substitutions.

In some embodiments, the anti-PD-L1 antibody comprises a full-length antibody comprising a heavy chain variable region (V_{H}) and a light chain variable region (V_{L}); wherein a) the V_{H} comprises a HC-CDR1, a HC-CDR2, and a HC-CDR3, respectively comprising the amino acid sequences of a CDR1, a CDR2, and a CDR3 within a heavy variable region (V_{H}) having the sequence set forth in SEQ ID No: 12, or a variant thereof having at least about 80% (including for example at least about any of 80%, 85%, 90%, 95%, 96%, 97%, 98%, or 99%) sequence identity to the sequence set forth in SEQ ID NO: 12; and b) the V_{L} comprises a LC-CDR1, a LC-CDR2, and a LC-CDR3, respectively comprising the amino acid sequences of a CDR1, a CDR2, and a CDR3 within a light chain variable region (V_{L}) having the sequence set forth in SEQ ID NO: 13, or a variant thereof having at least about 80% (including for example at least about any of 80%, 85%, 90%, 95%, 96%, 97%, 98%, or 99%) sequence identity to the sequence set forth in SEQ ID NO: 13.

In some embodiments, the anti-PD-L1 antibody comprises a full-length antibody comprising a heavy chain variable region (V_{H}) and a light chain variable region (V_{L}), wherein the V_{H} comprises the sequence set forth in SEQ ID No: 12, or a variant thereof having at least about 80% (including for example at least about any of 80%, 85%, 90%, 95%, 96%, 97%, 98%, or 99%) sequence identity to the sequence set forth in SEQ ID NO: 12; and/or the V_{L} comprises the sequence set forth in SEQ ID NO: 13, or a variant thereof having at least about 80% (including for example at least about any of 80%, 85%, 90%, 95%, 96%, 97%, 98%, or 99%) sequence identity to the sequence set forth in SEQ ID NO: 13.

In some embodiments, the amino acid substitutions described above are limited to “exemplary substitutions” shown in Table 2 of this application. In some embodiments, the amino acid substitutions are limited to “preferred substitutions” shown in Table 2 of this application.

### Antibody Moieties (e.g., Anti-PD-L1 Antibody Moiety)

The antibody moieties described herein (such as anti-PD-L1 antibody moiety) can have any one or more of the features described below.

In some embodiments, the antibody moiety comprises an Fc fragment. In some embodiments, the antibody moiety comprises a scFv. In some embodiments the antibody moiety comprises a scFv fused to an Fc fragment. In some embodiments, the antibody moiety comprises a scFv fused to an Fc fragment via a peptide linker. In some embodiments, the Fc fragment is a human IgG1 Fc fragment. In some embodiments, the Fc fragment comprises one or more mutations to increase clearance or decrease half-life.

In some embodiments, the Fc fragment comprises an immunoglobulin IgG heavy chain constant region comprising a hinge region (starting at Cys226), an IgG CH2 domain and CH3 domain. The term “hinge region” or “hinge sequence” as used herein refers to the amino acid sequence located between the linker and the CH2 domain. In some embodiments, the fusion protein comprises an Fc fragment comprising a hinge region. In some embodiments, the Fc fragment of the fusion protein starts at the hinge region and extends to the C-terminus of the IgG heavy chain. In some embodiments, the fusion protein comprises an Fc fragment that does not comprise the hinge region.

In some embodiments, the antibody moiety comprises an Fc fragment selected from the group consisting of Fc fragments from IgG, IgA, IgD, IgE, IgM, and combinations and hybrids thereof. In some embodiments, the Fc fragment is derived from a human IgG. In some embodiments, the Fc fragment comprises the Fc region of human IgG1, IgG2, IgG3, IgG4, or a combination or hybrid IgG. In some embodiments, the Fc fragment is an IgG1 Fc fragment. In some embodiments, the Fc fragment comprises the CH2 and CH3 domains of IgG1. In some embodiments, the Fc fragment is an IgG4 Fc fragment. In some embodiments, the Fc fragment comprises the CH2 and CH3 domains of IgG4. IgG4 Fc is known to exhibit less effector activity than IgG1 Fc, and thus may be desirable for some applications. In some embodiments, the Fc fragment is derived from of a mouse immunoglobulin.

In some embodiments, the IgG CH2 domain starts at Ala231. In some embodiments, the CH3 domain starts at Gly341. It is understood that the C-terminus Lys residue of human IgG can be optionally absent. It is also understood that conservative amino acid substitutions of the Fc region without affecting the desired structure and/or stability of Fc is contemplated within the scope of the invention.

Heterodimerization of non-identical polypeptides in the Fc fragments of the antibody moieties can be facilitated by methods known in the art, including without limitation, heterodimerization by the knob-into-hole technology. The structure and assembly method of the knob-into-hole technology can be found in, e.g., U.S. Pat. Nos. 5,821,333, 7,642,228, US 2011/0287009 and PCT/US2012/059810, hereby incorporated by reference in their entireties. This technology was developed by introducing a “knob” (or a protuberance) by replacing a small amino acid residue with a large one in the CH3 domain of one Fc and introducing a “hole” (or a cavity) in the CH3 domain of the other Fc by replacing one or more large amino acid residues with smaller ones. In some embodiments, one chain of the Fc fragment in the fusion protein comprises a knob, and the second chain of the Fc fragment comprises a hole.

The preferred residues for the formation of a knob are generally naturally occurring amino acid residues and are preferably selected from arginine (R), phenylalanine (F), tyrosine (Y) and tryptophan (W). Most preferred are tryptophan and tyrosine. In one embodiment, the original residue for the formation of the knob has a small side chain volume, such as alanine, asparagine, aspartic acid, glycine, serine, threonine or valine. Exemplary amino acid substitutions in the CH3 domain for forming the knob include without limitation the T366W, T366Y or F405W substitution.

The preferred residues for the formation of a hole are usually naturally occurring amino acid residues and are preferably selected from alanine (A), serine (S), threonine (T) and valine (V). In one embodiment, the original residue for the formation of the hole has a large side chain volume, such as tyrosine, arginine, phenylalanine or tryptophan. Exemplary amino acid substitutions in the CH3 domain for generating the hole include without limitation the T366S, L368A, F405A, Y407A, Y407T and Y407V substitutions. In certain embodiments, the knob comprises T366W substitution, and the hole comprises the T366S/L368A/Y407V substitutions. It is understood that other modifications to the Fc region known in the art that facilitate heterodimerization are also contemplated and encompassed by the instant application.

Other antibody moiety variants comprises any of the variants described herein (e.g., Fc variants, effector function variants, glycosylation variants, cysteine engineered variants), or combinations thereof, are contemplated.

a) Antibody Affinity

Binding specificity of the antibody moieties can be determined experimentally by methods known in the art. Such methods comprise, but are not limited to Western blots, ELISA-, RIA-, ECL-, IRMA-, EIA-, BIACORE™-tests and peptide scans.

In some embodiments, the K_{D} of the binding between the antibody moiety and the target antigen (e.g., PD-L1) is about 10⁻⁷ M to about 10⁻¹² M, about 10⁻⁷ M to about 10⁻⁸ M, about 10⁻⁸ M to about 10⁻⁹ M, about 10⁻⁹ M to about 10⁻¹⁰ M, about 10⁻¹⁰ M to about 10⁻¹¹ M, about 10⁻¹¹ M to about 10⁻¹² M, about 10⁻⁷ M to about 10⁻¹² M, about 10⁻⁸ M to about 10⁻¹² M, about 10⁻⁹ M to about 10⁻¹² M, about 10⁻¹⁰ M to about 10⁻¹² M, about 10⁻⁷ M to about 10⁻¹¹ M, about 10⁻⁸ M to about 10⁻¹¹ M, about 10⁻⁹ M to about 10⁻¹¹ M, about 10⁻⁷ M to about 10⁻¹⁰ M, about 10⁻⁸ M to about 10⁻¹⁰ M, or about 10⁻⁷ M to about 10⁻⁹ M in some embodiments, the K_{D} of the binding between the antibody moiety and the target antigen (e.g., PD-L1) is stronger than about any one of 10⁻⁷ M, 10⁻⁸ M, 10⁻⁹ M, 10⁻¹⁰ M, 10⁻¹¹ M, or 10⁻¹² M. In some embodiments, the target antigen (e.g., PD-L1) is a human antigen.

In some embodiments, the Kₒₙ of the binding between the antibody moiety and the target antigen (e.g., PD-L1) is about 10³ M⁻¹s⁻¹ to about 10⁸ M⁻¹s⁻¹, about 10³ M⁻¹s⁻¹ to about 10⁴ M⁻¹s⁻¹, about 10⁴ M⁻¹s⁻¹ to about 10⁵ M⁻¹s⁻¹, about 10⁵ M⁻¹s⁻¹ to about 10⁶ M⁻¹s⁻¹, about 10⁶ M⁻¹s⁻¹ to about 10⁷ M⁻¹s⁻¹, or about 10⁷ M⁻¹s⁻¹ to about 10⁸ M⁻¹s⁻¹. In some embodiments, the Kₒₙ of the binding between the antibody moiety and the target antigen (e.g., PD-L1) is about 10³ M⁻¹s⁻¹ to about 10⁵ M⁻¹s⁻¹, about 10⁴ M⁻¹s⁻¹ to about 10⁶ M⁻¹s⁻¹ about 10⁵ M⁻¹s⁻¹ to about 10⁷ M⁻¹s⁻¹, about 10⁶ M⁻¹s⁻¹ to about 10⁸ M⁻¹s⁻¹, about 10⁴ M⁻¹s⁻¹ to about 10⁷ M⁻¹s⁻¹, or about 10⁵ M⁻¹s⁻¹ to about 10⁸ some embodiments, the Kₒₙ of the binding between the antibody moiety and the target antigen (e.g., PD-L1) is no more than about any one of 10³ M⁻¹s⁻¹, 10⁴ M⁻¹s⁻¹, 10⁵ M⁻¹s⁻¹, 10⁶ M⁻¹s⁻¹, 10⁷ M⁻¹s⁻¹ or 10⁸ M⁻¹s⁻¹. In some embodiments, the target antigen (e.g., PD-L1) is human antigen.

In some embodiments, the K_{off} of the binding between the antibody moiety and the target antigen (e.g., PD-L1) is about 1 s⁻¹ to about 10⁻⁶s⁻¹, about 1 s⁻¹ to about 10⁻²s⁻¹, about 10⁻²s⁻¹ to about 10⁻³s⁻¹, about 10⁻³s⁻¹ to about 10⁻⁴s⁻¹, about 10⁻⁴s⁻¹ to about 10⁻⁵s⁻¹, about 10⁻⁵s⁻¹ to about 10⁻⁶s⁻¹, about 1 s⁻¹ to about 10⁻⁵s⁻¹, about 10⁻²s⁻¹ to about 10⁻⁶s⁻¹, about 10⁻³s⁻¹ to about 10⁻⁶s⁻¹, about 10⁻⁴s⁻¹ to about 10⁻⁶s⁻¹, about 10⁻²s⁻¹ to about 10⁻⁵s⁻¹, or about 10⁻³s⁻¹ to about 10⁻⁵s⁻¹. In some embodiments, the K_{off} of the binding between the antibody moiety and the target antigen (e.g., PD-L1) is at least about any one of 1 s⁻¹, 10⁻² s⁻¹, 10⁻³s⁻¹, 10⁻⁴s⁻¹, 10⁻⁵s⁻¹ or 10⁻⁶s⁻¹. In some embodiments, the target antigen (e.g., PD-L1) is human antigen.

b) Chimeric or Humanized Antibodies

In some embodiments, the antibody moiety is a chimeric antibody. Certain chimeric antibodies are described, e.g., in U.S. Pat. No. 4,816,567; and Morrison et al., Proc. Natl. Acad. Sci. USA, 81:6851-6855 (1984)). In some embodiments, a chimeric antibody comprises a non-human variable region (e.g., a variable region derived from mouse) and a human constant region. In some embodiments, a chimeric antibody is a “class switched” antibody in which the class or subclass has been changed from that of the parent antibody. Chimeric antibodies include antigen-binding fragments thereof.

In some embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing SDR (a-CDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to: framework regions selected using the “best-fit” method (see, e.g., Sims et al. J. Immunol. 151:2296 (1993)); Framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

c) Human Antibodies

In some embodiments, the antibody moiety is a human antibody (known as human domain antibody, or human DAb). Human antibodies can be produced using various techniques known in the art. Human antibodies are described generally in van Dijk and van de Winkel, Curr. Opin. Pharmoacol. 5: 368-74 (2001), Lonberg, Curr. Opin. Immunol. 20:450-459 (2008), and Chen, Mol. Immunol. 47(4):912-21 (2010). Transgenic mice or rats capable of producing fully human single-domain antibodies (or DAb) are known in the art. See, e.g., US20090307787A1, U.S. Pat. No. 8,754,287, US20150289489A1, US20100122358A1, and WO2004049794.

Human antibodies (e.g., human DAbs) may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE™ technology; U.S. Pat. No. 5,770,429 describing HuMAB® technology; U.S. Pat. No. 7,041,870 describing K-M MOUSE® technology, and U.S. Patent Application Publication No. US 2007/0061900, describing VELOCIMOUSE® technology). Human variable regions from intact antibodies generated by such animals may be further modified, e.g., by combining with a different human constant region.

Human antibodies (e.g., human DAbs) can also be made by hybridoma-based methods. Human myeloma and mouse-human heteromyeloma cell lines for the production of human monoclonal antibodies have been described (See, e.g., Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991)). Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, XiandaiMianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3):185-91 (2005).

Human antibodies (e.g., human DAbs) may also be generated by isolating Fv clone variable domain sequences selected from human-derived phage display libraries. Such variable domain sequences may then be combined with a desired human constant domain. Techniques for selecting human antibodies from antibody libraries are described below.

d) Library-Derived Antibodies

The antibody moieties may be isolated by screening combinatorial libraries for antibodies with the desired activity or activities. For example, a variety of methods are known in the art for generating phage display libraries and screening such libraries for antibodies possessing the desired binding characteristics. Such methods are reviewed, e.g., in Hoogenboom et al. in Methods in Molecular Biology 178:1-37 (O'Brien et al., ed., Human Press, Totowa, N.J., 2001) and further described, e.g., in the McCafferty et al., Nature 348:552-554; Clackson et al., Nature 352: 624-628 (1991); Marks et al., J. Mol. Biol. 222: 581-597 (1992); Marks and Bradbury, in Methods in Molecular Biology 248:161-175 (Lo, ed., Human Press, Totowa, N.J., 2003); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132(2004). Methods for constructing single-domain antibody libraries have been described, for example, see U.S. Pat. No. 7,371,849.

In certain phage display methods, repertoires of V_{H} and V_{L} genes are separately cloned by polymerase chain reaction (PCR) and recombined randomly in phage libraries, which can then be screened for antigen-binding phage as described in Winter et al., Ann. Rev. Immunol., 12: 433-455 (1994). Phage typically displays antibody fragments, either as scFv fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self-antigens without any immunization as described by Griffiths et al., EMBO J, 12: 725-734 (1993). Finally, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter, J. Mol. Biol., 227: 381-388 (1992). Patent publications describing human antibody phage libraries include, for example: U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360.

Antibodies or antibody fragments isolated from human antibody libraries are considered human antibodies or human antibody fragments herein.

e) Substitution, Insertion, Deletion and Variants

In some embodiments, antibody variants having one or more amino acid substitutions are provided. Sites of interest for substitutional mutagenesis include the HVRs (or CDRs) and FRs. Conservative substitutions are shown in Table 2 under the heading of “Preferred substitutions.” More substantial changes are provided in Table 2 under the heading of “exemplary substitutions,” and as further described below in reference to amino acid side chain classes. Amino acid substitutions may be introduced into an antibody of interest and the products screened for a desired activity, e.g., retained/improved antigen binding, decreased immunogenicity, or improved ADCC or CDC.

TABLE 2
Amino acid substitutions

| Original | | Preferred |
|---|---|---|
| Residue | Exemplary Substitutions | Substitutions |
| Ala (A) | Val; Leu; Ile | Val |
| Arg (R) | Lys; Gin; Asn | Lys |
| Asn (N) | Gin; His; Asp, Lys; Arg | Gin |
| Asp (D) | Glu; Asn | Glu |
| Cys (C) | Ser; Ala | Ser |
| Gin (Q) | Asn; Glu | Asn |
| Glu (E) | Asp; Gin | Asp |
| Gly (G) | Ala | Ala |
| His (H) | Asn; Gin; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gin; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; and (6) aromatic: Trp, Tyr, Phe.

Non-conservative substitutions will entail exchanging a member of one of these classes for another class.

One type of substitutional variant involves substituting one or more hypervariable region residues of a parent antibody (e.g., a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques such as those described herein. Briefly, one or more HVR residues are mutated and the variant antibodies displayed on phage and screened for a particular biological activity (e.g. binding affinity).

Alterations (e.g., substitutions) may be made in HVRs, e.g., to improve antibody affinity. Such alterations may be made in HVR “hotspots,” i.e., residues encoded by codons that undergo mutation at high frequency during the somatic maturation process (see, e.g., Chowdhury, Methods Mol. Biol. 207:179-196 (2008)), and/or SDRs (a-CDRs), with the resulting variant V_{H} or V_{L} being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, N.J., (2001)). In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves HVR-directed approaches, in which several HVR residues (e.g., 4-6 residues at a time) are randomized. HVR residues involved in antigen binding may be specifically identified, e.g., using alanine scanning mutagenesis or modeling. CDR-H3 and CDR-L3 in particular are often targeted.

In some embodiments, substitutions, insertions, or deletions may occur within one or more HVRs so long as such alterations do not substantially reduce the ability of the antibody to bind antigen. For example, conservative alterations (e.g., conservative substitutions as provided herein) that do not substantially reduce binding affinity may be made in HVRs. Such alterations may be outside of HVR “hotspots” or CDRs. In some embodiments of the variant V_{H}H sequences provided above, each HVR either is unaltered, or contains no more than one, two or three amino acid substitutions.

A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as Arg, Asp, His, Lys, and Glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for ADEPT) or a polypeptide which increases the serum half-life of the antibody.

f) Glycosylation Variants

In some embodiments, the antibody moiety is altered to increase or decrease the extent to which the antibody moiety is glycosylated. Addition or deletion of glycosylation sites to an antibody may be conveniently accomplished by altering the amino acid sequence such that one or more glycosylation sites is created or removed.

Where the antibody moiety comprises an Fc region, the carbohydrate attached thereto may be altered. Native antibodies produced by mammalian cells typically comprise a branched, biantennary oligosaccharide that is generally attached by an N-linkage to Asn297 of the C_{H}2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in the antibody moiety may be made in order to create antibody variants with certain improved properties.

In some embodiments, the antibody moiety has a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g., complex, hybrid and high mannose structures) as measured by MALDI-TOF mass spectrometry, as described in WO 2008/077546, for example. Asn297 refers to the asparagine residue located at about position 297 in the Fc region (EU numbering of Fc region residues); however, Asn297 may also be located about ±3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87: 614 (2004). Examples of cell lines capable of producing defucosylated antibodies include Lec13 CHO cells deficient in protein fucosylation (Ripka et al. Arch. Biochem. Biophys. 249:533-545 (1986); US Patent Application No. US 2003/0157108 A1, Presta, L; and WO 2004/056312 A1, Adams et al., especially at Example 11), and knockout cell lines, such as alpha-1,6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94(4):680-688 (2006); and WO2003/085107).

In some embodiments, the antibody moiety has bisected oligosaccharides, e.g., in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878 (Jean-Mairet et al.); U.S. Pat. No. 6,602,684 (Umana et al.); and US 2005/0123546 (Umana et al.). Antibody variants with at least one galactose residue in the oligosaccharide attached to the Fc region are also provided. Such antibody variants may have improved CDC function. Such antibody variants are described, e.g., in WO 1997/30087 (Patel et al.); WO 1998/58964 (Raju, S.); and WO 1999/22764 (Raju, S.).

g) Fc Region Variants

In some embodiments, one or more amino acid modifications may be introduced into the Fc region of the antibody moiety, thereby generating an Fc region variant. The Fc region variant may comprise a human Fc region sequence (e.g., a human IgG1, IgG2, IgG3 or IgG4 Fc region) comprising an amino acid modification (e.g. a substitution) at one or more amino acid positions.

In some embodiments, the Fc fragment possesses some but not all effector functions, which make it a desirable candidate for applications in which the half-life of the antibody moiety in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express FcγRIII only, whereas monocytes express FcγRI, FcγRII and FcγRIII FcR expression on hematopoietic cells is summarized in Table 2 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g. Hellstrom, I. et al. Proc. Nat'l Acad. Sci. USA 83:7059-7063 (1986)) and Hellstrom, I et al., Proc. Nat'l Acad. Sci. USA 82:1499-1502 (1985); 5,821,337 (see Bruggemann, M. et al., J. Exp. Med. 166:1351-1361 (1987)). Alternatively, non-radioactive assays methods may be employed (see, for example, ACTI™ non-radioactive cytotoxicity assay for flow cytometry (CellTechnology, Inc. Mountain View, Calif.; and CytoTox 96 non-radioactive cytotoxicity assay (Promega, Madison, Wis.). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. Proc. Nat'l Acad. Sci. USA 95:652-656 (1998). C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)).FcRn binding and in vivo clearance/half-life determinations can also be performed using methods known in the art (see, e.g., Petkova, S. B. et al., Int'l. Immunol. 18(12):1759-1769 (2006)).

Antibodies with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Certain antibody variants with improved or diminished binding to FcRs are described. (See, e.g., U.S. Pat. No. 6,737,056; WO 2004/056312, and Shields et al., J. Biol. Chem. 9(2): 6591-6604 (2001).)

In some embodiments, the Fc fragment is an IgG1 Fc fragment. In some embodiments, the IgG1 Fc fragment comprises a L234A mutation and/or a L235Amutation. In some embodiments, the Fc fragment is an IgG2 or IgG4 Fc fragment. In some embodiments, the Fc fragment is an IgG4 Fc fragment comprising a S228P, F234A, and/or a L235A mutation.

In some embodiments, the antibody moiety comprises an Fc region with one or more amino acid substitutions which improve ADCC, e.g., substitutions at positions 298, 333, and/or 334 of the Fc region (EU numbering of residues).

In some embodiments, alterations are made in the Fc region that result in altered (i.e., either improved or diminished) C1q binding and/or Complement Dependent Cytotoxicity (CDC), e.g., as described in U.S. Pat. No. 6,194,551, WO 99/51642, and Idusogie et al. J. Immunol. 164: 4178-4184 (2000).

In some embodiments, the antibody moiety variant comprising a variant Fc region comprising one or more amino acid substitutions which alters half-life and/or changes binding to the neonatal Fc receptor (FcRn). Antibodies with increased half-lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934A1 (Hinton et al.). Those antibodies comprise an Fc region with one or more substitutions therein which alters binding of the Fc region to FcRn. Such Fc variants include those with substitutions at one or more of Fc region residues, e.g., substitution of Fc region residue 434 (U.S. Pat. No. 7,371,826).

See also Duncan & Winter, Nature 322:738-40 (1988); U.S. Pat. Nos. 5,648,260; 5,624,821; and WO 94/29351 concerning other examples of Fc region variants.

h) Cysteine Engineered Antibody Variants

In some embodiments, it may be desirable to create cysteine engineered antibody moieties, e.g., “thioMAbs,” in which one or more residues of an antibody are substituted with cysteine residues. In particular embodiments, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. In some embodiments, any one or more of the following residues may be substituted with cysteine: A118 (EU numbering) of the heavy chain; and S400 (EU numbering) of the heavy chain Fc region. Cysteine engineered antibody moieties may be generated as described, e.g., in U.S. Pat. No. 7,521,541.

i) Antibody Derivatives

In some embodiments, the antibody moiety described herein may be further modified to comprise additional nonproteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer are attached, they can be the same or different molecules. In general, the number and/or type of polymers used for derivatization can be determined based on considerations including, but not limited to, the particular properties or functions of the antibody to be improved, whether the antibody derivative will be used in diagnosis under defined conditions, etc.

In some embodiments, the antibody moiety may be further modified to comprise one or more biologically active protein, polypeptides or fragments thereof. “Bioactive” or “biologically active”, as used herein interchangeably, means showing biological activity in the body to carry out a specific function. For example, it may mean the combination with a particular biomolecule such as protein, DNA, etc., and then promotion or inhibition of the activity of such biomolecule. In some embodiments, the bioactive protein or fragments thereof include proteins and polypeptides that are administered to patients as the active drug substance for prevention of or treatment of a disease or condition, as well as proteins and polypeptides that are used for diagnostic purposes, such as enzymes used in diagnostic tests or in vitro assays, as well as proteins and polypeptides that are administered to a patient to prevent a disease such as a vaccine.

### Linkers

In some embodiments, the fusion proteins described herein comprise one or more linkers between two moieties (e.g., the TGFβ superfamily receptor ectodomain and the anti-PD-L1 antibody moiety described above). The length, the degree of flexibility and/or other properties of the linker(s) used in the fusion proteins may have some influence on properties, including but not limited to the affinity, specificity or avidity for one or more particular antigens or epitopes. For example, longer linkers may be selected to ensure that two adjacent domains do not sterically interfere with one another. In some embodiment, a linker (such as peptide linker) comprises flexible residues (such as glycine and serine) so that the adjacent domains are free to move relative to each other. For example, a glycine-serine doublet can be a suitable peptide linker. In some embodiments, the linker is a non-peptide linker. In some embodiments, the linker is a peptide linker. In some embodiments, the linker is a non-cleavable linker. In some embodiments, the linker is a cleavable linker.

Other linker considerations include the effect on physical or pharmacokinetic properties of the resulting compound, such as solubility, lipophilicity, hydrophilicity, hydrophobicity, stability (more or less stable as well as planned degradation), rigidity, flexibility, immunogenicity, modulation of antibody binding, the ability to be incorporated into a micelle or liposome, and the like.

In some embodiments, the anti-PD-L1 antibody moiety comprises a full-length antibody. In some embodiments, the TGFβ superfamily receptor ectodomain is fused to the full length antibody via a linker.

In some embodiments, the linker is a peptide linker as described below. In some embodiments, the peptide linker has a length of about one to about fifty, about two to about fourth, about three to about thirty, or about four to about twenty amino acids.

In some embodiments, the linker is a cleavable linker. In some embodiments, the linker is a non-cleavable linker.

In some embodiments, the linker is a GS linker.

In some embodiments, the linker comprises an amino acid sequence of any one of SEQ ID NOs: 72-80 and 90-96. In some embodiments, the linker comprises an amino acid sequence of any one of SEQ ID NOs: 72-80. In some embodiments, the linker comprises an amino acid sequence of any one of SEQ ID NOs: 72-77.

### Peptide Linkers

The peptide linker may have a naturally occurring sequence, or a non-naturally occurring sequence. For example, a sequence derived from the hinge region of heavy chain only antibodies may be used as the linker. See, for example, WO1996/34103.

The peptide linker can be of any suitable length. In some embodiments, the peptide linker is at least about any of 1, 2, 3, 4, 5, 6, 7, 8, 9, 10, 11, 12, 13, 14, 15, 16, 17, 18, 19, 20, 25, 30, 35, 40, 50, 75, 100 or more amino acids long. In some embodiments, the peptide linker is no more than about any of 100, 75, 50, 40, 35, 30, 25, 20, 19, 18, 17, 16, 15, 14, 13, 12, 11, 10, 9, 8, 7, 6, 5 or fewer amino acids long. In some embodiments, the length of the peptide linker is any of about 1 amino acid to about 10 amino acids, about 1 amino acid to about 20 amino acids, about 1 amino acid to about 30 amino acids, about 5 amino acids to about 15 amino acids, about 10 amino acids to about 25 amino acids, about 5 amino acids to about 30 amino acids, about 10 amino acids to about 30 amino acids long, about 30 amino acids to about 50 amino acids, about 50 amino acids to about 100 amino acids, or about 1 amino acid to about 100 amino acids.

An essential technical feature of such peptide linker is that said peptide linker does not comprise any polymerization activity. The characteristics of a peptide linker, which comprise the absence of the promotion of secondary structures, are known in the art and described, e.g., in Dall′Acqua et al. (Biochem. (1998) 37, 9266-9273), Cheadle et al. (Mol Immunol (1992) 29, 21-30) and Raag and Whitlow (FASEB (1995) 9(1), 73-80). A particularly preferred amino acid in context of the “peptide linker” is Gly. Furthermore, peptide linkers that also do not promote any secondary structures are preferred. The linkage of the domains to each other can be provided by, e.g., genetic engineering. Methods for preparing fusion proteins and polypeptides and expressing them in mammalian cells or bacteria are well-known in the art (e.g. WO 99/54440, Ausubel, Current Protocols in Molecular Biology, Green Publishing Associates and Wiley Interscience, N. Y. 1989 and 1994 or Sambrook et al., Molecular Cloning: A Laboratory Manual, Cold Spring Harbor Laboratory Press, Cold Spring Harbor, N. Y., 2001).

The peptide linker can be a stable linker, which is not cleavable by proteases, especially by Matrix metalloproteinases (MMPs).

The linker can also be a flexible linker. Exemplary flexible linkers include glycine polymers (G)ₙ (SEQ ID NO: 85), glycine-serine polymers (including, for example, (GS)ₙ (SEQ ID NO: 86), (GSGGS)ₙ (SEQ ID NO: 87), (GGGGS)ₙ (SEQ ID NO: 82), and (GGGS)ₙ (SEQ ID NO: 88), where n is an integer of at least one), glycine-alanine polymers, alanine-serine polymers, and other flexible linkers known in the art. Glycine and glycine-serine polymers are relatively unstructured, and therefore may be able to serve as a neutral tether between components. Glycine accesses significantly more phi-psi space than even alanine, and is much less restricted than residues with longer side chains (see Scheraga, Rev. Computational Chem. 11 173-142 (1992)). The ordinarily skilled artisan will recognize that design of an antibody fusion protein can include linkers that are all or partially flexible, such that the linker can include a flexible linker portion as well as one or more portions that confer less flexible structure to provide a desired antibody fusion protein structure. In some embodiments, the linker is a GS linker.

In some embodiments, the peptide linker comprises the hinge region of an IgG, such as the hinge region of human IgG1. In some embodiments, the peptide linker comprises the hinge region of an IgG, such as the hinge region of human IgG1. In some embodiments, the peptide linker comprises a modified sequence derived from the hinge region of an IgG. In some embodiments, the linker comprises an amino acid sequence of any one of SEQ ID NOs: 57-58 and 63-68.

In some embodiments, the TGFβ superfamily receptor ectodomain and the anti-PD-L1 antibody moiety are linked together by a linker of sufficient length to enable the TGFβ superfamily receptor ectodomain and anti-PD-L1 antigen binding domain to fold in such a way as to permit binding to TGFβ (such as TGFβ1 or TGFβ3) and PD-L1. In some embodiments, the linker has the amino acid sequence of (GGGGS)ₙ(SEQ ID NO: 82), wherein n is an integer between 1 and 8, e.g. (GGGGS)₆ (SEQ ID NO: 83; hereinafter referred to as “(G₄S)6” or “GS6”). In some embodiments, the peptide linker comprise the amino acid sequence of (GSTSGSGKPGSGEGS)ₙ (SEQ ID NO: 84), wherein n is an integer between 1 and 3.

In some embodiments, the linker has an amino acid sequence selected from the group consisting of SEQ ID NOs: 56-68 and 82-88. In some embodiments, the linker has an amino acid sequence selected from the group consisting of SEQ ID NOs: 56-68.

### Non-Peptide Linkers

Coupling of two moieties may be accomplished by any chemical reaction that will bind the two molecules so long as both components retain their respective activities, e.g., binding to TGFβ (e.g., TGFβ1 and/or TGFβ3) and PD-L1, respectively. This linkage can include many chemical mechanisms, for instance covalent binding, affinity binding, intercalation, coordinate binding and complexation. In some embodiments, the binding is covalent binding. Covalent binding can be achieved either by direct condensation of existing side chains or by the incorporation of external bridging molecules. Many bivalent or polyvalent linking agents may be useful in coupling protein molecules in this context. For example, representative coupling agents can include organic compounds such as thioesters, carbodiimides, succinimide esters, diisocyanates, glutaraldehyde, diazobenzenes and hexamethylene diamines. This listing is not intended to be exhaustive of the various classes of coupling agents known in the art but, rather, is exemplary of the more common coupling agents (see Killen and Lindstrom, Jour. Immun. 133:1335-2549 (1984); Jansen et al., Immunological Reviews 62:185-216 (1982); and Vitetta et al., Science 238:1098 (1987)).

Linkers the can be applied in the present application are described in the literature (see, for example, Ramakrishnan, S. et al., Cancer Res. 44:201-208 (1984) describing use of MBS (M-maleimidobenzoyl-N-hydroxysuccinimide ester). In some embodiments, non-peptide linkers used herein include: (i) EDC (1-ethyl-3-(3-dimethylamino-propyl) carbodiimide hydrochloride; (ii) SMPT (4-succinimidyloxycarbonyl-alpha-methyl-alpha-(2-pridyl-dithio)-toluene (Pierce Chem. Co., Cat. (21558G); (iii) SPDP (succinimidyl-6 [3-(2-pyridyldithio) propionamido] hexanoate (Pierce Chem. Co., Cat #21651G); (iv) Sulfo-LC-SPDP (sulfosuccinimidyl 6 [3-(2-pyridyldithio)-propianamide] hexanoate (Pierce Chem. Co. Cat. #2165-G); and (v) sulfo-NHS (N-hydroxysulfo-succinimide: Pierce Chem. Co., Cat. #24510) conjugated to EDC.

The linkers described above contain components that have different attributes, thus may lead to fusion proteins with differing physio-chemical properties. For example, sulfo-NHS esters of alkyl carboxylates are more stable than sulfo-NHS esters of aromatic carboxylates. NHS-ester containing linkers are less soluble than sulfo-NHS esters. Further, the linker SMPT contains a sterically hindered disulfide bond, and can form antibody fusion protein with increased stability. Disulfide linkages, are in general, less stable than other linkages because the disulfide linkage is cleaved in vitro, resulting in less antibody fusion protein available. Sulfo-NHS, in particular, can enhance the stability of carbodimide couplings. Carbodimide couplings (such as EDC) when used in conjunction with sulfo-NHS, forms esters that are more resistant to hydrolysis than the carbodimide coupling reaction alone.

### Nucleic Acids

Nucleic acid molecules encoding the various fusion proteins, polypeptides, or antibody moieties described herein are also contemplated. In some embodiments, there is provided a nucleic acid (or a set of nucleic acids) encoding one or more of the fusion proteins, polypeptide, or antibody moieties described herein or a portion thereof.

Also contemplated here are isolated host cell comprising a fusion protein or polypeptide, an isolated nucleic acid encoding the polypeptide or the polypeptide components of the fusion protein, or a vector comprising a nucleic acid encoding the polypeptide or polypeptide components of the fusion protein described herein.

The present application also includes variants to these nucleic acid sequences. For example, the variants include nucleotide sequences that hybridize to the nucleic acid sequences encoding polypeptide or the fusion protein described herein under at least moderately stringent hybridization conditions.

The present application also provides vectors in which a nucleic acid of the present application is inserted.

The nucleic acids of the present invention may also be used for nucleic acid immunization and gene therapy, using standard gene delivery protocols. Methods for gene delivery are known in the art. See, e.g., U.S. Pat. Nos. 5,399,346, 5,580,859, 5,589,466, incorporated by reference herein in their entireties. In some embodiments, the invention provides a gene therapy vector.

The nucleic acid can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the expression vector may be provided to a cell in the form of a viral vector. Viral vector technology is well known in the art and is described, for example, in Sambrook et al. (2001, Molecular Cloning: A Laboratory Manual, Cold Spring Harbor Laboratory, New York), and in other virology and molecular biology manuals. Viruses which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers (see, e.g., WO 01/96584; WO 01/29058; and U.S. Pat. No.

### III. Methods of Preparation

In some embodiments, there is provided a method of preparing a fusion protein (e.g., fusion protein that comprises a TGFβ superfamily receptor ectodomain binds to PD-L1) or a polypeptide described herein. In some embodiments, there is provided a method of preparing a composition such as polynucleotide, nucleic acid construct, vector, host cell, or culture medium that is produced during the preparation of the fusion protein or the polypeptide. The fusion protein, polypeptide, or composition described herein may be prepared by a number of processes as generally described below and more specifically in the Examples.

In some embodiments, there is provided a method of producing any of the fusion proteins or polypeptides described herein, comprising: a) culturing the host cell expressing the fusion protein or a portion thereof, or the polypeptide, under conditions effective to express the fusion protein or a portion thereof, or the polypeptide; and b) obtaining the expressed fusion protein or a portion thereof, or the polypeptide from the host cell. In some embodiments, the method further comprises purifying the expressed fusion protein or a portion thereof, or polypeptide.

### Antibody Expression and Production

The antibody moieties (including anti-PD-L1 antibodies) described herein can be prepared using any known methods in the art, including those described below and in the Examples.

### Monoclonal Antibodies

Monoclonal antibodies are obtained from a population of substantially homogeneous antibodies, i.e., the individual antibodies comprising the population are identical except for possible naturally occurring mutations and/or post-translational modifications (e.g., isomerizations, amidations) that may be present in minor amounts. Thus, the modifier “monoclonal” indicates the character of the antibody as not being a mixture of discrete antibodies. For example, the monoclonal antibodies may be made using the hybridoma method first described by Kohler et al., Nature, 256:495 (1975), or may be made by recombinant DNA methods (U.S. Pat. No. 4,816,567). In the hybridoma method, a mouse or other appropriate host animal, such as a hamster or a llama, is immunized as hereinabove described to elicit lymphocytes that produce or are capable of producing antibodies that will specifically bind the protein used for immunization. Alternatively, lymphocytes may be immunized in vitro. Lymphocytes then are fused with myeloma cells using a suitable fusing agent, such as polyethylene glycol, to form a hybridoma cell (Goding, Monoclonal Antibodies: Principles and Practice, pp. 59-103 (Academic Press, 1986). Also see Example 1 for immunization in Camels.

The immunizing agent will typically include the antigenic protein or a fusion variant thereof. Generally, either peripheral blood lymphocytes (“PBLs”) are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell. Goding, Monoclonal Antibodies: Principles and Practice, Academic Press (1986), pp. 59-103.

Immortalized cell lines are usually transformed mammalian cells, particularly myeloma cells of rodent, bovine and human origin. Usually, rat or mouse myeloma cell lines are employed. The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which are substances that prevent the growth of HGPRT-deficient cells.

Preferred immortalized myeloma cells are those that fuse efficiently, support stable high-level production of antibody by the selected antibody-producing cells, and are sensitive to a medium such as HAT medium. Among these, preferred are murine myeloma lines, such as those derived from MOPC-21 and MPC-11 mouse tumors available from the Salk Institute Cell Distribution Center, San Diego, Calif. USA, and SP-2 cells (and derivatives thereof, e.g., X63-Ag8-653) available from the American Type Culture Collection, Manassas, Va. USA. Human myeloma and mouse-human heteromyeloma cell lines also have been described for the production of human monoclonal antibodies (Kozbor, J. Immunol., 133:3001 (1984); Brodeur et al., Monoclonal Antibody Production Techniques and Applications, pp. 51-63 (Marcel Dekker, Inc., New York, 1987)).

Culture medium in which hybridoma cells are growing is assayed for production of monoclonal antibodies directed against the antigen. Preferably, the binding specificity of monoclonal antibodies produced by hybridoma cells is determined by immunoprecipitation or by an in vitro binding assay, such as radioimmunoassay (RIA) or enzyme-linked immunosorbent assay (ELISA).

The culture medium in which the hybridoma cells are cultured can be assayed for the presence of monoclonal antibodies directed against the desired antigen. Preferably, the binding affinity and specificity of the monoclonal antibody can be determined by immunoprecipitation or by an in vitro binding assay, such as radioimmunoassay (RIA) or enzyme-linked assay (ELISA). Such techniques and assays are known in the in art. For example, binding affinity may be determined by the Scatchard analysis of Munson et al., Anal. Biochem., 107:220 (1980).

After hybridoma cells are identified that produce antibodies of the desired specificity, affinity, and/or activity, the clones may be subcloned by limiting dilution procedures and grown by standard methods (Goding, supra). Suitable culture media for this purpose include, for example, D-MEM or RPMI-1640 medium. In addition, the hybridoma cells may be grown in vivo as tumors in a mammal.

The monoclonal antibodies secreted by the subclones are suitably separated from the culture medium, ascites fluid, or serum by conventional immunoglobulin purification procedures such as, for example, protein A-Sepharose, hydroxylapatite chromatography, gel electrophoresis, dialysis, or affinity chromatography.

Monoclonal antibodies may also be made by recombinant DNA methods, such as those described in U.S. Pat. No. 4,816,567, and as described above. DNA encoding the monoclonal antibodies is readily isolated and sequenced using conventional procedures (e.g., by using oligonucleotide probes that are capable of binding specifically to genes encoding the heavy and light chains of murine antibodies). The hybridoma cells serve as a preferred source of such DNA. Once isolated, the DNA may be placed into expression vectors, which are then transfected into host cells such as E. coli cells, simian COS cells, Chinese hamster ovary (CHO) cells, or myeloma cells that do not otherwise produce immunoglobulin protein, in order to synthesize monoclonal antibodies in such recombinant host cells. Review articles on recombinant expression in bacteria of DNA encoding the antibody include Skerra et al., Curr. Opinion in Immunol., 5: 256-262 (1993) and Plückthun, Immunol. Revs. 130:151-188 (1992).

In a further embodiment, antibodies can be isolated from antibody phage libraries generated using the techniques described in McCafferty et al., Nature, 348:552-554 (1990). Clackson et al., Nature, 352:624-628 (1991) and Marks et al., J. Mol. Biol., 222:581-597 (1991) describe the isolation of murine and human antibodies, respectively, using phage libraries. Subsequent publications describe the production of high affinity (nM range) human antibodies by chain shuffling (Marks et al., Bio/Technology, 10:779-783 (1992)), as well as combinatorial infection and in vivo recombination as a strategy for constructing very large phage libraries (Waterhouse et al., Nucl. Acids Res., 21:2265-2266 (1993)). Thus, these techniques are viable alternatives to traditional monoclonal antibody hybridoma techniques for isolation of monoclonal antibodies.

The DNA also may be modified, for example, by substituting the coding sequence for human heavy- and light-chain constant domains in place of the homologous murine sequences (U.S. Pat. No. 4,816,567; Morrison, et al., Proc. Natl Acad. Sci. USA, 81:6851 (1984)), or by covalently joining to the immunoglobulin coding sequence all or part of the coding sequence for a non-immunoglobulin polypeptide. Typically, such non-immunoglobulin polypeptides are substituted for the constant domains of an antibody, or they are substituted for the variable domains of one antigen-combining site of an antibody to create a chimeric bivalent antibody comprising one antigen-combining site having specificity for an antigen and another antigen-combining site having specificity for a different antigen.

The monoclonal antibodies described herein may by monovalent, the preparation of which is well known in the art. For example, one method involves recombinant expression of immunoglobulin light chain and a modified heavy chain. The heavy chain is truncated generally at any point in the Fc region so as to prevent heavy chain crosslinking. Alternatively, the relevant cysteine residues may be substituted with another amino acid residue or are deleted so as to prevent crosslinking. In vitro methods are also suitable for preparing monovalent antibodies. Digestion of antibodies to produce fragments thereof, particularly Fab fragments, can be accomplished using routine techniques known in the art.

Chimeric or hybrid antibodies also may be prepared in vitro using known methods in synthetic protein chemistry, including those involving crosslinking agents. For example, immunotoxins may be constructed using a disulfide-exchange reaction or by forming a thioether bond. Examples of suitable reagents for this purpose include iminothiolate and methyl mercaptobutyrimidate.

### Nucleic Acid Molecules Encoding Fusion Proteins or Polypeptides

In some embodiments, there is provided a polynucleotide encoding any one of the fusion proteins (or a portion thereof) or polypeptides described herein. In some embodiments, there is provided a polynucleotide prepared using any one of the methods as described herein. In some embodiments, a nucleic acid molecule comprises a polynucleotide that encodes a heavy chain or a light chain of an antibody moiety (e.g., anti-PD-L1 antibody moiety, e.g., anti-PD-L1 full-length antibody). In some embodiments, a nucleic acid molecule comprises both a polynucleotide that encodes a heavy chain and a polynucleotide that encodes a light chain, of an antibody moiety (e.g., anti-PD-L1 antibody moiety, e.g., anti-PD-L1 full-length antibody). In some embodiments, a first nucleic acid molecule comprises a first polynucleotide that encodes a heavy chain and a second nucleic acid molecule comprises a second polynucleotide that encodes a light chain.

In some embodiments, the heavy chain and the light chain are expressed from one nucleic acid molecule, or from two separate nucleic acid molecules, as two separate polypeptides. In some embodiments, a single polynucleotide encodes a single polypeptide comprising both a heavy chain and a light chain linked together.

In some embodiments, a polynucleotide encoding a heavy chain or light chain of an antibody moiety (e.g., anti-PD-L1 antibody moiety, e.g., anti-PD-L1 full-length antibody) comprises a nucleotide sequence that encodes a leader sequence, which, when translated, is located at the N terminus of the heavy chain or light chain. As discussed above, the leader sequence may be the native heavy or light chain leader sequence, or may be another heterologous leader sequence.

In some embodiments, a nucleic acid molecule comprises a polynucleotide that encodes a TGFβR ectodomain polypeptide (such as any of the polypeptides described herein).

In some embodiments, the polynucleotide is a DNA. In some embodiments, the polynucleotide is an RNA. In some embodiments, the RNA is an mRNA. In some embodiments, the polynucleotide comprises a nucleic acid sequence having at least about 80% (such as at least about any one of 80%, 85%, 90%, 95%, 96%, 97%, 98%, or 99%) sequence identity to the nucleic acid sequence of any one of SEQ ID Nos: 19-21 and 38-53.

Nucleic acid molecules may be constructed using recombinant DNA techniques conventional in the art. In some embodiments, a nucleic acid molecule is an expression vector that is suitable for expression in a selected host cell.

### Nucleic Acid Construct

In some embodiments, there is provided a nucleic acid construct comprising any one of the polynucleotides described herein. In some embodiments, there is provided a nucleic acid construct prepared using any method described herein.

In some embodiments, the nucleic acid construct further comprises a promoter operably linked to the polynucleotide. In some embodiments, the polynucleotide corresponds to a gene, wherein the promoter is a wild-type promoter for the gene.

### Vectors

In some embodiments, there is provided a vector comprising any polynucleotides that encode any of the antibody moieties or fusion proteins described herein or nucleic acid construct described herein. In some embodiments, there is provided a vector prepared using any method described herein. Vectors comprising polynucleotides that encode any of the fusion proteins or polypeptides described herein are also provided. Such vectors include, but are not limited to, DNA vectors, phage vectors, viral vectors, retroviral vectors, etc. In some embodiments, a vector comprises a) a first polynucleotide sequence encoding a heavy chain or a light chain of an anti-PD-L1 full-length antibody fused with a TBFb superfamily receptor ectodomain, and b) a second polynucleotide sequence encoding a light chain or a heavy chain of an anti-PD-L1 full-length antibody that pairs with the first nucleotide. In some embodiments, the first vector and second vector are transfected into host cells in similar amounts (such as similar molar amounts or similar mass amounts). In some embodiments, a mole- or mass-ratio of between 5:1 and 1:5 of the first vector and the second vector is transfected into host cells. In some embodiments, a mass ratio of between 1:1 and 1:5 for the vector encoding the heavy chain and the vector encoding the light chain is used. In some embodiments, a mass ratio of 1:2 for the vector encoding the heavy chain and the vector encoding the light chain is used.

In some embodiments, a vector is selected that is optimized for expression of polypeptides in CHO or CHO-derived cells (e.g., CHO-3E7 cells), or in NSO cells. Exemplary such vectors are described, e.g., in Running Deer et al., Biotechnol. Prog. 20:880-889 (2004).

### Host Cells

In some embodiments, there is provided a host cell comprising any polypeptide, nucleic acid construct and/or vector described herein. In some embodiments, there is provided a host cell prepared using any method described herein. In some embodiments, the host cell is capable of producing any of antibody moieties or fusion proteins described herein under a fermentation condition.

In some embodiments, the fusion proteins and polypeptides described herein may be expressed in prokaryotic cells, such as bacterial cells; or in eukaryotic cells, such as fungal cells (such as yeast), plant cells, insect cells, and mammalian cells. Such expression may be carried out, for example, according to procedures known in the art. Exemplary eukaryotic cells that may be used to express polypeptides include, but are not limited to, COS cells, including COS 7 cells; 293 cells, including 293-6E cells; CHO cells, including CHO—S, CHO-3E7, DG44. Lec13 CHO cells, and FUT8 CHO cells; PER.C6® cells (Crucell); and NSO cells. In some embodiments, the antibody moieties and fusion proteins described herein may be expressed in yeast. See, e.g., U.S. Publication No. US 2006/0270045 A1. In some embodiments, a particular eukaryotic host cell is selected based on its ability to make desired post-translational modifications to the heavy chains and/or light chains of the antibody moiety. For example, in some embodiments, CHO cells produce polypeptides that have a higher level of sialylation than the same polypeptide produced in 293 cells.

Introduction of one or more nucleic acids into a desired host cell may be accomplished by any method, including but not limited to, calcium phosphate transfection, DEAE-dextran mediated transfection, cationic lipid-mediated transfection, electroporation, transduction, infection, etc. Non-limiting exemplary methods are described, e.g., in Sambrook et al., Molecular Cloning, A Laboratory Manual, 3^{rd} ed. Cold Spring Harbor Laboratory Press (2001). Nucleic acids may be transiently or stably transfected in the desired host cells, according to any suitable method.

The invention also provides host cells comprising any of the polynucleotides or vectors described herein. In some embodiments, the invention provides a host cell comprising a fusion protein or polypeptide described herein or a portion thereof. Any host cells capable of over-expressing heterologous DNAs can be used for the purpose of isolating the genes encoding the antibody, polypeptide or protein of interest. Non-limiting examples of mammalian host cells include but not limited to COS, HeLa, and CHO cells. See also PCT Publication No. WO 87/04462. Suitable non-mammalian host cells include prokaryotes (such as E. coli or B. subtillis) and yeast (such as S. cerevisae, S. pombe; or K. lactis).

### Culture Medium

In some embodiments, there is provided a culture medium comprising any antibody moiety, polynucleotide, fusion protein, nucleic acid construct, vector, and/or host cell described herein. In some embodiments, there is provided a culture medium prepared using any method described herein.

In some embodiments, the medium comprises hypoxanthine, aminopterin, and/or thymidine (e.g., HAT medium). In some embodiments, the medium does not comprise serum. In some embodiments, the medium comprises serum. In some embodiments, the medium is a D-MEM or RPMI-1640 medium.

### Purification of Fusion Proteins or Polypeptides

The fusion proteins or polypeptides described herein may be purified by any suitable method. Such methods include, but are not limited to, the use of affinity matrices or hydrophobic interaction chromatography. Suitable affinity ligands include the ROR1 ECD and ligands that bind antibody constant regions. For example, a Protein A, Protein G, Protein A/G, or an antibody affinity column may be used to bind the constant region and to purify a fusion protein comprising an Fc fragment. Hydrophobic interactive chromatography, for example, a butyl or phenyl column, may also suitable for purifying some polypeptides such as antibodies. Ion exchange chromatography (e.g. anion exchange chromatography and/or cation exchange chromatography) may also suitable for purifying some polypeptides such as antibodies. Mixed-mode chromatography (e.g. reversed phase/anion exchange, reversed phase/cation exchange, hydrophilic interaction/anion exchange, hydrophilic interaction/cation exchange, etc.) may also suitable for purifying some polypeptides such as antibodies. Many methods of purifying polypeptides are known in the art.

### V. Methods of Treatments

Also provided here are methods of treating a disease or condition in an individual. The methods comprise administering a fusion protein or polypeptide described herein into individuals (e.g., mammals such as humans).

In some embodiments, there is provided a method of treating a disease or condition (e.g., cancer) in an individual, comprising administering to the individual an effective amount of a fusion protein comprising a) a full-length antibody that specifically binds to PD-L1 and comprises two heavy chains and two light chains, wherein each of the two heavy chains comprises a heavy chain variable region (V_{H}) and each of the two light chains comprises a light chain variable region (V_{L}); b) a TGFβ superfamily receptor ectodomain; wherein the TGFβ superfamily receptor ectodomain is fused to at least one or both of the heavy chains or light chains of the anti-PD-L1 antibody. In some embodiments, full-length antibody comprises Fc fragment derived from a human IgG1. In some embodiments, the Fc fragment derived from human IgG1 comprises a K214R mutation and an A297N mutation. In some embodiments, the TGFβ superfamily receptor ectodomain comprises a TGFβ receptor type II ectodomain or a variant thereof. In some embodiments, the TGFβ receptor type II ectodomain or a variant thereof comprises a human TGFβ receptor type II ectodomain or a variant thereof. In some embodiments, the human TGFβ receptor type II ectodomain or a variant thereof comprises a variant human TGFβ receptor type II ectodomain that has at least about 90% sequence identity to SEQ ID NO: 2, wherein the variant comprises one or more amino acid substitution at position N47, N71, and/or N131 according to the numbering of SEQ ID NO: 2. In some embodiments, the variant comprises the amino acid sequence of SEQ ID NO: 3. In some embodiments, the TGFβ superfamily receptor ectodomain comprises a TGFβ receptor type I ectodomain or a variant thereof. In some embodiments, the TGFβ receptor type I ectodomain or a variant thereof comprises a human TGFβ receptor type I ectodomain or a variant thereof. In some embodiments, the human TGFβ receptor type I ectodomain or a variant thereof comprises an amino acid sequence of SEQ ID NO: 1 or a variant that has at least about 90% sequence identity. In some embodiments, the TGFβ superfamily receptor ectodomain is fused to at least one of the heavy chains or light chains of the anti-PD-L1 antibody via a linker. In some embodiments, the linker is a peptide linker. In some embodiments, the peptide linker has a length of about 2 to about 25 amino acids (such as about 2 to about 10 amino acids). In some embodiments, the linker comprises the amino acid sequence of SEQ ID NOs: 56-68.

In some embodiments, there is provided a method of treating a disease or condition (e.g., cancer) in an individual, comprising administering to the individual an effective amount of a fusion protein comprising a) a full-length antibody that specifically binds to PD-L1 and comprises two heavy chains and two light chains, wherein each of the two heavy chains comprises a heavy chain variable region (V_{H}) and each of the two light chains comprises a light chain variable region (V_{L}); b) a TGFβ superfamily receptor ectodomain; wherein the TGFβ superfamily receptor ectodomain is fused to at least one or both of the light chains of the anti-PD-L1 antibody. In some embodiments, the TGFβ superfamily receptor ectodomain is fused to the C-terminus of the at least one or both of the two light chains. In some embodiments, the TGFβ superfamily receptor ectodomain is fused to the N-terminus of the at least one or both of the two light chains. In some embodiments, the TGFβ superfamily receptor ectodomain is fused to both the N-terminus and the C-terminus of the at least one or both of the two light chains. In some embodiments, full-length antibody comprises Fc fragment derived from a human IgG1. In some embodiments, the Fc fragment derived from human IgG1 comprises a K214R mutation and an A297N mutation. In some embodiments, the TGFβ superfamily receptor ectodomain comprises a TGFβ receptor type II ectodomain or a variant thereof. In some embodiments, the TGFβ receptor type II ectodomain or a variant thereof comprises a human TGFβ receptor type II ectodomain or a variant thereof. In some embodiments, the human TGFβ receptor type II ectodomain or a variant thereof comprises a variant human TGFβ receptor type II ectodomain that has at least about 90% sequence identity to SEQ ID NO: 2, wherein the variant comprises one or more amino acid substitution at position N47, N71, and/or N131 according to the numbering of SEQ ID NO: 2. In some embodiments, the variant comprises the amino acid sequence of SEQ ID NO: 3. In some embodiments, the TGFβ superfamily receptor ectodomain comprises a TGFβ receptor type I ectodomain or a variant thereof. In some embodiments, the TGFβ receptor type I ectodomain or a variant thereof comprises a human TGFβ receptor type I ectodomain or a variant thereof. In some embodiments, the human TGFβ receptor type I ectodomain or a variant thereof comprises an amino acid sequence of SEQ ID NO: 1 or a variant that has at least about 90% sequence identity. In some embodiments, the TGFβ superfamily receptor ectodomain is fused to at least one of the heavy chains or light chains of the anti-PD-L1 antibody via a linker. In some embodiments, the linker is a peptide linker. In some embodiments, the peptide linker has a length of about 2 to about 25 amino acids (such as about 2 to about 10 amino acids). In some embodiments, the linker comprises the amino acid sequence of SEQ ID NOs: 56-68.

In some embodiments, there is provided a method of treating a disease or condition (e.g., cancer) in an individual, comprising administering to the individual an effective amount of a fusion protein comprising a) a full-length antibody that specifically binds to PD-L1 and comprises two heavy chains and two light chains, wherein each of the two heavy chains comprises a heavy chain variable region (V_{H}) and each of the two light chains comprises a light chain variable region (V_{L}); b) a TGFβ superfamily receptor ectodomain; wherein the TGFβ superfamily receptor ectodomain is fused to at least one or both of the heavy chains of the anti-PD-L1 antibody. In some embodiments, the TGFβ superfamily receptor ectodomain is fused to the C-terminus of the at least one or both of the two heavy chains. In some embodiments, the TGFβ superfamily receptor ectodomain is fused to the N-terminus of the at least one or both of the two heavy chains. In some embodiments, the TGFβ superfamily receptor ectodomain is fused to both the N-terminus and the C-terminus of the at least one or both of the two heavy chains. In some embodiments, full-length antibody comprises Fc fragment derived from a human IgG1. In some embodiments, the Fc fragment derived from human IgG1 comprises a K214R mutation and an A297N mutation. In some embodiments, the TGFβ superfamily receptor ectodomain comprises a TGFβ receptor type II ectodomain or a variant thereof. In some embodiments, the TGFβ receptor type II ectodomain or a variant thereof comprises a human TGFβ receptor type II ectodomain or a variant thereof. In some embodiments, the human TGFβ receptor type II ectodomain or a variant thereof comprises a variant human TGFβ receptor type II ectodomain that has at least about 90% sequence identity to SEQ ID NO: 2, wherein the variant comprises one or more amino acid substitution at position N47, N71, and/or N131 according to the numbering of SEQ ID NO: 2. In some embodiments, the variant comprises the amino acid sequence of SEQ ID NO: 3. In some embodiments, the TGFβ superfamily receptor ectodomain comprises a TGFβ receptor type I ectodomain or a variant thereof. In some embodiments, the TGFβ receptor type I ectodomain or a variant thereof comprises a human TGFβ receptor type I ectodomain or a variant thereof. In some embodiments, the human TGFβ receptor type I ectodomain or a variant thereof comprises an amino acid sequence of SEQ ID NO: 1 or a variant that has at least about 90% sequence identity. In some embodiments, the TGFβ superfamily receptor ectodomain is fused to at least one of the heavy chains or light chains of the anti-PD-L1 antibody via a linker. In some embodiments, the linker is a peptide linker. In some embodiments, the peptide linker has a length of about 2 to about 25 amino acids (such as about 2 to about 10 amino acids). In some embodiments, the linker comprises the amino acid sequence of SEQ ID NOs: 56-68.

In some embodiments, there is provided a method of treating a disease or condition (e.g., cancer) in an individual, comprising administering to the individual an effective amount of a fusion protein comprising a) a full-length antibody that specifically binds to PD-L1 and comprises two heavy chains and two light chains, wherein each of the two heavy chains comprises a heavy chain variable region (V_{H}) and each of the two light chains comprises a light chain variable region (V_{L}); b) a TGFβ superfamily receptor ectodomain; wherein the TGFβ superfamily receptor ectodomain is fused to i) at least one or both of the light chains of the anti-PD-L1 antibody and ii) at least one or both of the heavy chains of the anti-PD-L1 antibody. In some embodiments, the TGFβ superfamily receptor ectodomain is fused to the C-terminus of i) the two heavy chains and ii) two light chains. In some embodiments, the TGFβ superfamily receptor ectodomain is fused to the N-terminus of i) the two heavy chains and ii) two light chains. In some embodiments, the TGFβ superfamily receptor ectodomain is fused to i) the N-terminus of the two heavy chains and ii) the C-terminus of two light chains. In some embodiments, the TGFβ superfamily receptor ectodomain is fused to i) the C-terminus of the two heavy chains and ii) the N-terminus of two light chains. In some embodiments, full-length antibody comprises Fc fragment derived from a human IgG1. In some embodiments, the Fc fragment derived from human IgG1 comprises a K214R mutation and an A297N mutation. In some embodiments, the TGFβ superfamily receptor ectodomain comprises a TGFβ receptor type II ectodomain or a variant thereof. In some embodiments, the TGFβ receptor type II ectodomain or a variant thereof comprises a human TGFβ receptor type II ectodomain or a variant thereof. In some embodiments, the human TGFβ receptor type II ectodomain or a variant thereof comprises a variant human TGFβ receptor type II ectodomain that has at least about 90% sequence identity to SEQ ID NO: 2, wherein the variant comprises one or more amino acid substitution at position N47, N71, and/or N131 according to the numbering of SEQ ID NO: 2. In some embodiments, the variant comprises the amino acid sequence of SEQ ID NO: 3. In some embodiments, the TGFβ superfamily receptor ectodomain comprises a TGFβ receptor type I ectodomain or a variant thereof. In some embodiments, the TGFβ receptor type I ectodomain or a variant thereof comprises a human TGFβ receptor type I ectodomain or a variant thereof. In some embodiments, the human TGFβ receptor type I ectodomain or a variant thereof comprises an amino acid sequence of SEQ ID NO: 1 or a variant that has at least about 90% sequence identity. In some embodiments, the TGFβ superfamily receptor ectodomain is fused to at least one of the heavy chains or light chains of the anti-PD-L1 antibody via a linker. In some embodiments, the linker is a peptide linker. In some embodiments, the peptide linker has a length of about 2 to about 25 amino acids (such as about 2 to about 10 amino acids). In some embodiments, the linker comprises the amino acid sequence of SEQ ID NOs: 56-68.

In some embodiments, there is provided a method of treating a disease or condition (e.g., cancer) in an individual, comprising administering to the individual an effective amount of a fusion protein comprising a) a full-length antibody that specifically binds to PD-L1 and comprises two heavy chains and two light chains, wherein each of the two heavy chains comprises a heavy chain variable region (V_{H}) and each of the two light chains comprises a light chain variable region (V_{L}); b) a first TGFβ superfamily receptor ectodomain comprising a TGFβ receptor type I ectodomain or a variant thereof; and c) a second TGFβ superfamily receptor ectodomain comprising a TGFβ receptor type II ectodomain or a variant thereof, and wherein the first and the second TGFβ superfamily receptor ectodomains are both fused to one or both of the two heavy chains of the anti-PD-L1 antibody. In some embodiments, both of the first and the second TGFβ superfamily receptor ectodomains are fused to the N-terminus of the one or both of the two heavy chains of the anti-PD-L1 antibody. In some embodiments, both of the first and the second TGFβ superfamily receptor ectodomains are fused to the C-terminus of the one or both of the two heavy chains of the anti-PD-L1 antibody. In some embodiments, the first and the second TGFβ superfamily receptor ectodomains are linked to each other via a linker (such as any of the linkers described herein, such as a linker comprising an amino acid sequence of any of SEQ ID NOs: 56-68). In some embodiments, one of the first and the second TGFβ superfamily receptor ectodomains is fused to N-terminus of the one or both of the two heavy chains of the anti-PD-L1 antibody, and the other one of the first and the second TGFβ superfamily receptor ectodomains is fused to C-terminus of the one or both of the two heavy chains of the anti-PD-L1 antibody. In some embodiments, the TGFβ receptor type I ectodomain or a variant thereof is fused to N-terminus of the one or both of the two heavy chains of the anti-PD-L1 antibody; and the TGFβ receptor type II ectodomain or a variant thereof is fused to C-terminus of the one or both of the two heavy chains of the anti-PD-L1 antibody. In some embodiments, the TGFβ receptor type I ectodomain or a variant thereof is fused to C-terminus of the one or both of the two heavy chains of the anti-PD-L1 antibody; and the TGFβ receptor type II ectodomain or a variant thereof is fused to N-terminus of the one or both of the two heavy chains of the anti-PD-L1 antibody. In some embodiments, full-length antibody comprises Fc fragment derived from a human IgG1. In some embodiments, the Fc fragment derived from human IgG1 comprises a K214R mutation and an A297N mutation. In some embodiments, the TGFβ superfamily receptor ectodomain comprises a TGFβ receptor type II ectodomain or a variant thereof. In some embodiments, the TGFβ receptor type II ectodomain or a variant thereof comprises a human TGFβ receptor type II ectodomain or a variant thereof. In some embodiments, the human TGFβ receptor type II ectodomain or a variant thereof comprises a variant human TGFβ receptor type II ectodomain that has at least about 90% sequence identity to SEQ ID NO: 2, wherein the variant comprises one or more amino acid substitution at position N47, N71, and/or N131 according to the numbering of SEQ ID NO: 2. In some embodiments, the variant comprises the amino acid sequence of SEQ ID NO: 3. In some embodiments, the TGFβ superfamily receptor ectodomain comprises a TGFβ receptor type I ectodomain or a variant thereof. In some embodiments, the TGFβ receptor type I ectodomain or a variant thereof comprises a human TGFβ receptor type I ectodomain or a variant thereof. In some embodiments, the human TGFβ receptor type I ectodomain or a variant thereof comprises an amino acid sequence of SEQ ID NO: 1 or a variant that has at least about 90% sequence identity. In some embodiments, the TGFβ superfamily receptor ectodomain is fused to at least one of the heavy chains or light chains of the anti-PD-L1 antibody via a linker. In some embodiments, the linker is a peptide linker. In some embodiments, the peptide linker has a length of about 2 to about 25 amino acids (such as about 2 to about 10 amino acids). In some embodiments, the linker comprises the amino acid sequence of SEQ ID NOs: 56-68.

In some embodiments, there is provided a method of treating a disease or condition (e.g., cancer) in an individual, comprising administering to the individual an effective amount of a fusion protein comprising a) a full-length antibody that specifically binds to PD-L1 and comprises two heavy chains and two light chains, wherein each of the two heavy chains comprises a heavy chain variable region (V_{H}) and each of the two light chains comprises a light chain variable region (V_{L}); b) a first TGFβ superfamily receptor ectodomain comprising a TGFβ receptor type I ectodomain or a variant thereof; and c) a second TGFβ superfamily receptor ectodomain comprising a TGFβ receptor type II ectodomain or a variant thereof, and wherein the first and the second TGFβ superfamily receptor ectodomains are both fused to one or both of the two light chains of the anti-PD-L1 antibody. In some embodiments, both of the first and the second TGFβ superfamily receptor ectodomains are fused to the N-terminus of the one or both of the two light chains of the anti-PD-L1 antibody. In some embodiments, both of the first and the second TGFβ superfamily receptor ectodomains are fused to the C-terminus of the one or both of the two light chains of the anti-PD-L1 antibody. In some embodiments, the first and the second TGFβ superfamily receptor ectodomains are linked to each other via a linker (such as any of the linkers described herein, such as a linker comprising an amino acid sequence of any of SEQ ID NOs: 56-68). In some embodiments, one of the first and the second TGFβ superfamily receptor ectodomains is fused to N-terminus of the one or both of the two light chains of the anti-PD-L1 antibody, and the other one of the first and the second TGFβ superfamily receptor ectodomains is fused to C-terminus of the one or both of the two light chains of the anti-PD-L1 antibody. In some embodiments, the TGFβ receptor type I ectodomain or a variant thereof is fused to N-terminus of the one or both of the two light chains of the anti-PD-L1 antibody; and the TGFβ receptor type II ectodomain or a variant thereof is fused to C-terminus of the one or both of the two light chains of the anti-PD-L1 antibody. In some embodiments, the TGFβ receptor type I ectodomain or a variant thereof is fused to C-terminus of the one or both of the two light chains of the anti-PD-L1 antibody; and the TGFβ receptor type II ectodomain or a variant thereof is fused to N-terminus of the one or both of the two light chains of the anti-PD-L1 antibody. In some embodiments, full-length antibody comprises Fc fragment derived from a human IgG1. In some embodiments, the Fc fragment derived from human IgG1 comprises a K214R mutation and an A297N mutation. In some embodiments, the TGFβ superfamily receptor ectodomain comprises a TGFβ receptor type II ectodomain or a variant thereof. In some embodiments, the TGFβ receptor type II ectodomain or a variant thereof comprises a human TGFβ receptor type II ectodomain or a variant thereof. In some embodiments, the human TGFβ receptor type II ectodomain or a variant thereof comprises a variant human TGFβ receptor type II ectodomain that has at least about 90% sequence identity to SEQ ID NO: 2, wherein the variant comprises one or more amino acid substitution at position N47, N71, and/or N131 according to the numbering of SEQ ID NO: 2. In some embodiments, the variant comprises the amino acid sequence of SEQ ID NO: 3. In some embodiments, the TGFβ superfamily receptor ectodomain comprises a TGFβ receptor type I ectodomain or a variant thereof. In some embodiments, the TGFβ receptor type I ectodomain or a variant thereof comprises a human TGFβ receptor type I ectodomain or a variant thereof. In some embodiments, the human TGFβ receptor type I ectodomain or a variant thereof comprises an amino acid sequence of SEQ ID NO: 1 or a variant that has at least about 90% sequence identity. In some embodiments, the TGFβ superfamily receptor ectodomain is fused to at least one of the heavy chains or light chains of the anti-PD-L1 antibody via a linker. In some embodiments, the linker is a peptide linker. In some embodiments, the peptide linker has a length of about 2 to about 25 amino acids (such as about 2 to about 10 amino acids). In some embodiments, the linker comprises the amino acid sequence of SEQ ID NOs: 56-68.

In some embodiments, there is provided a method of treating a disease or condition (e.g., cancer) in an individual, comprising administering to the individual an effective amount of a fusion protein comprising a) a full-length antibody that specifically binds to PD-L1 and comprises two heavy chains and two light chains, wherein each of the two heavy chains comprises a heavy chain variable region (V_{H}) and each of the two light chains comprises a light chain variable region (V_{L}); b) a first TGFβ superfamily receptor ectodomain; and c) a second TGFβ superfamily receptor ectodomain, wherein the first TGFβ superfamily receptor ectodomain is fused to one or both of the two light chains of the anti-PD-L1 antibody, and wherein the second TGFβ superfamily receptor ectodomain is fused to one or both of the two heavy chains of the anti-PD-L1 antibody. In some embodiments, the first TGFβ superfamily receptor ectodomain is fused to N-terminus of one or both of the two light chains of the anti-PD-L1 antibody, and the second TGFβ superfamily receptor ectodomain is fused to N-terminus of one or both of the two heavy chains of the anti-PD-L1 antibody. In some embodiments, the first TGFβ superfamily receptor ectodomain is fused to C-terminus of one or both of the two light chains of the anti-PD-L1 antibody, and the second TGFβ superfamily receptor ectodomain is fused to C-terminus of one or both of the two heavy chains of the anti-PD-L1 antibody. In some embodiments, the first TGFβ superfamily receptor ectodomain is fused to N-terminus of one or both of the two light chains of the anti-PD-L1 antibody, and the second TGFβ superfamily receptor ectodomain is fused to C-terminus of one or both of the two heavy chains of the anti-PD-L1 antibody. In some embodiments, the first TGFβ superfamily receptor ectodomain is fused to C-terminus of one or both of the two light chains of the anti-PD-L1 antibody, and the second TGFβ superfamily receptor ectodomain is fused to N-terminus of one or both of the two heavy chains of the anti-PD-L1 antibody. In some embodiments, the first TGFβ receptor ectodomain comprises a TGFβ receptor type I ectodomain or a variant thereof, and the second TGFβ receptor ectodomain comprises a TGFβ receptor type II ectodomain or a variant thereof. In some embodiments, the first TGFβ receptor ectodomain comprises a TGFβ receptor type II ectodomain or a variant thereof, and the second TGFβ receptor ectodomain comprises a TGFβ receptor type I ectodomain or a variant thereof. In some embodiments, full-length antibody comprises Fc fragment derived from a human IgG1. In some embodiments, the Fc fragment derived from human IgG1 comprises a K214R mutation and an A297N mutation. In some embodiments, the TGFβ superfamily receptor ectodomain comprises a TGFβ receptor type II ectodomain or a variant thereof. In some embodiments, the TGFβ receptor type II ectodomain or a variant thereof comprises a human TGFβ receptor type II ectodomain or a variant thereof. In some embodiments, the human TGFβ receptor type II ectodomain or a variant thereof comprises a variant human TGFβ receptor type II ectodomain that has at least about 90% sequence identity to SEQ ID NO: 2, wherein the variant comprises one or more amino acid substitution at position N47, N71, and/or N131 according to the numbering of SEQ ID NO: 2. In some embodiments, the variant comprises the amino acid sequence of SEQ ID NO: 3. In some embodiments, the TGFβ superfamily receptor ectodomain comprises a TGFβ receptor type I ectodomain or a variant thereof. In some embodiments, the TGFβ receptor type I ectodomain or a variant thereof comprises a human TGFβ receptor type I ectodomain or a variant thereof. In some embodiments, the human TGFβ receptor type I ectodomain or a variant thereof comprises an amino acid sequence of SEQ ID NO: 1 or a variant that has at least about 90% sequence identity. In some embodiments, the TGFβ superfamily receptor ectodomain is fused to at least one of the heavy chains or light chains of the anti-PD-L1 antibody via a linker. In some embodiments, the linker is a peptide linker. In some embodiments, the peptide linker has a length of about 2 to about 25 amino acids (such as about 2 to about 10 amino acids). In some embodiments, the linker comprises the amino acid sequence of SEQ ID NOs: 56-68.

In some embodiments, there is also provided a method of overcoming or reducing resistance of an immune checkpoint inhibitor (such as an anti-PD-L1 antibody) comprising administering any of the fusion proteins or polypeptides described herein.

As described herein, the fusion proteins of the present application have exhibited lower TGFβ2 binding affinity, which potentially result in a better safety profile (e.g., more moderate toxicity associated with TGFβ2 neutralization) as compared to methods that involve a reference fusion protein (such as M7824). In some embodiments, the methods described herein are used in patients with cardiac diseases to minimize cardiac toxicity resulted from the treatment.

In some embodiments, the side effects associated with TGFβ2 neutralization after treatment are to a lesser extent as compared to a corresponding treatment that involves a reference fusion protein (such as M7824). In some embodiments, the side effects associated with TGFβ2 neutralization comprise cardiac toxicity associated with TGFβ2 neutralization. In some embodiments, the cardiac toxicity can be measured by assessing a) chest pain, b) heart rhythm changes, c) fatigue, d) shortness of breath, e) weight gain, and/or f) swelling before and after treatment.

### Disease or Condition

The fusion proteins or polypeptides described herein can be used for treating any disease or condition. In some embodiments, the disease or condition is a cancer. In some embodiments, the disease or condition is or is derived from an infectious disease (such as a virus infection).

In some embodiments, the fusion protein or polypeptide is used in a method for treating a cancer. Cancers that may be treated using any of the methods described herein include tumors that are not vascularized, or not yet substantially vascularized, as well as vascularized tumors. Types of cancers to be treated with the fusion proteins as described in this application include, but are not limited to, carcinoma, blastoma, sarcoma, benign and malignant tumors, and malignancies e.g., sarcomas, carcinomas, and melanomas. Adult tumors/cancers and pediatric tumors/cancers are also included. In some embodiments, the cancer is a solid tumor. In some embodiments, the cancer is a hematological malignancy.

Examples of cancers that may be treated by the methods of this application include, but are not limited to, anal cancer, astrocytoma (e.g., cerebellar and cerebral), basal cell carcinoma, bladder cancer, bone cancer, (osteosarcoma and malignant fibrous histiocytoma), brain tumor (e.g., glioma, brain stem glioma, cerebellar or cerebral astrocytoma (e.g., astrocytoma, malignant glioma, medulloblastoma, and glioblastoma), breast cancer, cervical cancer, colon cancer, colorectal cancer, endometrial cancer (e.g., uterine cancer), esophageal cancer, eye cancer (e.g., intraocular melanoma and retinoblastoma), gastric (stomach) cancer, gastrointestinal stromal tumor (GIST), head and neck cancer (such as head and neck squamous cancer), hepatocellular (liver) cancer (e.g., hepatic carcinoma and heptoma), liver cancer, biliary tract cancer (such as cholangiocarcinoma and/or gallbladder cancer), prostate cancer, lung cancer (e.g., small cell lung cancer, non-small cell lung cancer, adenocarcinoma of the lung, and squamous carcinoma of the lung), medulloblastoma, melanoma, mesothelioma, myelodysplastic syndromes, nasopharyngeal cancer, neuroblastoma, ovarian cancer, pancreatic cancer, parathyroid cancer, cancer of the peritoneal, pituitary tumor, rectal cancer, renal cancer, renal pelvis and ureter cancer (transitional cell cancer), rhabdomyosarcoma, skin cancer (e.g., non-melanoma (e.g., squamous cell carcinoma), melanoma, and Merkel cell carcinoma), small intestine cancer, squamous cell cancer, testicular cancer, thyroid cancer, and tuberous sclerosis. Additional examples of cancers can be found in The Merck Manual of Diagnosis and Therapy, 19th Edition, § on Hematology and Oncology, published by Merck Sharp & Dohme Corp., 2011 (ISBN 978-0-911910-19-3); The Merck Manual of Diagnosis and Therapy, 20th Edition, § on Hematology and Oncology, published by Merck Sharp & Dohme Corp., 2018 (ISBN 978-0-911-91042-1) (2018 digital online edition at internet website of Merck Manuals); and SEER Program Coding and Staging Manual 2016, each of which are incorporated by reference in their entirety for all purposes.

In some embodiments, the cancer is non-small cell lung cancer (NSCLC), small cell lung cancer (SCLC), cervical cancer, oropharyngeal cancer, anal cancer, vaginal or penile cancer, biliary tract cancer, cholangiocarcinoma, gallbladder cancer, head and neck cancer, pancreas cancer, prostate cancer, urothelial cancer, bladder cancer, genitourinary cancer, urogenital cancer, gastric cancer, breast cancer or colorectal cancer.

In some embodiments, the cancer is Her2 positive breast cancer. In some embodiments, the cancer is triple negative breast cancer (such as metastatic triple negative breast cancer). In some embodiments, the cancer is metastatic hormone receptor positive, Her2 negative breast cancer.

In some embodiments, the cancer is associated with human papillomavirus (HPV).

In various embodiments, the cancer is early stage cancer, non-metastatic cancer, primary cancer, advanced cancer, locally advanced cancer, metastatic cancer, cancer in remission, recurrent cancer, cancer in an adjuvant setting, cancer in a neoadjuvant setting, or cancer substantially refractory to a therapy.

In some embodiments, the disease or condition is or is derived from an infectious disease (such as a viral infection). In some embodiments, the disease or condition is Recurrent Respiratory Papillomatosis. In some embodiment, the virus is human papilloma virus (HPV).

In some embodiments, the diseased tissue has an abnormal expression of PD-L1. For example the expression of PD-L1 in the diseased tissue is at least about 5%, 10%, 20%, 30%, 40%, 50%, 60%, 70%, 80%, 90%, 100%, 125%, 150%, 175%, 200%, 300%, 400%, or 500% more than the PD-L1 expression level in a reference tissue. In some embodiments, the reference tissue is the corresponding tissue derived from an individual who does not have the disease or condition. In some embodiments, the PD-L1 expression level in a reference tissue is measured by averaging the PD-L1 expression level in reference tissues in a group of individuals who do not have the disease or condition.

### Dosing and Method of Administration

The dose of the fusion protein or polypeptide described herein used for treating a disease or disorder as described herein administered into the individual may vary with the particular fusion protein or polypeptide, the mode of administration, and the type of disease or condition being treated. In some embodiments, the type of disease or condition is a cancer. In some embodiments, the effective amount of the fusion protein or polypeptide is an amount that is effective to result in an objective response (such as a partial response or a complete response). In some embodiments, the effective amount of the fusion protein or polypeptide is an amount that is sufficient to result in a complete response in the individual. In some embodiments, the effective amount of the fusion protein or polypeptide is an amount that is sufficient to result in a partial response in the individual. In some embodiments, the effective amount of the fusion protein or polypeptide is an amount that is sufficient to produce an overall response rate of more than about any of 20%, 25%, 30%, 35%, 40%, 45%, 50%, 55%, 60%, 64%, 65%, 70%, 75%, 80%, 85%, or 90% among a population of individuals treated with the fusion protein or polypeptide. Responses of an individual to the treatment of the methods described herein can be determined, for example, based on RECIST levels.

In some embodiments, the effective amount of the fusion protein or polypeptide is an amount that is sufficient to prolong progress-free survival of the individual. In some embodiments, the effective amount of the fusion protein or polypeptide is an amount that is sufficient to prolong overall survival of the individual. In some embodiments, the effective amount of the fusion protein or polypeptide is an amount that is sufficient to produce clinical benefit of more than about any of 50%, 60%, 70%, or 77% among a population of individuals treated with the fusion protein or polypeptide.

In some embodiments, the effective amount of the fusion protein or polypeptide alone or in combination with a second, third, and/or fourth agent, is an amount sufficient to decrease the size of a tumor, decrease the number of cancer cells, or decrease the growth rate of a tumor by at least about any of 10%, 20%, 30%, 40%, 50%, 60%, 70%, 80%, 90%, 95% or 100% compared to the corresponding tumor size, number of cancer cells, or tumor growth rate in the same subject prior to treatment or compared to the corresponding activity in other subjects not receiving the treatment (e.g., receiving a placebo treatment). Standard methods can be used to measure the magnitude of this effect, such as in vitro assays with purified enzyme, cell-based assays, animal models, or human testing.

In some embodiments, the effective amount of the fusion protein or polypeptide is an amount that is below the level that induces a toxicological effect (i.e., an effect above a clinically acceptable level of toxicity) or is at a level where a potential side effect can be controlled or tolerated when the composition is administered to the individual.

In some embodiments, the effective amount of the fusion protein or polypeptide is an amount that is close to a maximum tolerated dose (MTD) of the composition following the same dosing regimen. In some embodiments, the effective amount of the fusion protein or polypeptide is more than about any of 80%, 90%, 95%, or 98% of the MTD.

In some embodiments, the effective amount of the fusion protein or polypeptide is an amount that slows or inhibits the progression of the disease or condition (for example, by at least about 5%, 10%, 15%, 20%, 30%, 40%, 50%) as compared to that of the individual not receiving the treatment.

In some embodiments, the effective amount of the fusion protein or polypeptide is an amount that reduces the side effects (auto-immune response) of a condition (e.g., transplantation) (for example, by at least about 5%, 10%, 15%, 20%, 30%, 40%, or 50%) as compared to that of the individual not receiving the treatment.

In some embodiments of any of the above aspects, the effective amount of the fusion protein or polypeptide is in the range of about 0.001 μg/kg to about 100 mg/kg of total body weight, for example, about 0.005 μg/kg to about 50 mg/kg, about 0.01 μg/kg to about 10 mg/kg, or about 0.01 μg/kg to about 1 mg/kg.

In some embodiments, the treatment comprises more than one administration of any one of the fusion proteins or polypeptides (such as about two, three, four, five, six, seven, eight, night, or ten administrations of the fusion protein or polypeptide). In some embodiments, two administrations are carried out within about a week. In some embodiments, a second administration is carried out at least about 1, 2, 3, 4, 5, 6, or 7 days after the completion of the first administration.

The fusion protein can be administered to an individual (such as human) via various routes, including, for example, intravenous, intra-arterial, intraperitoneal, intrapulmonary, oral, inhalation, intravesicular, intramuscular, intra-tracheal, subcutaneous, intraocular, intrathecal, transmucosal, and transdermal. In some embodiments, the fusion protein is included in a pharmaceutical composition while administered into the individual. In some embodiments, sustained continuous release formulation of the composition may be used. In some embodiments, the composition is administered intravenously. In some embodiments, the composition is administered intraperitoneally. In some embodiments, the composition is administered intravenously. In some embodiments, the composition is administered intraperitoneally. In some embodiments, the composition is administered intramuscularly. In some embodiments, the composition is administered subcutaneously. In some embodiments, the composition is administered intravenously. In some embodiments, the composition is administered orally.

### Combination Therapy

This application also provides methods of administering any one of fusion protein or polypeptide into an individual for treating a disease or condition (such as cancer), wherein the method further comprises administering a second agent or therapy. In some embodiments, the second agent or therapy is a standard or commonly used agent or therapy for treating the disease or condition. In some embodiments, the second agent or therapy comprises a chemotherapeutic agent. In some embodiments, the second agent or therapy comprises a surgery. In some embodiments, the second agent or therapy comprises a radiation therapy. In some embodiments, the second agent or therapy comprises an immunotherapy. In some embodiments, the second agent or therapy comprises a hormonal therapy. In some embodiments, the second agent or therapy comprises a tyrosine kinase inhibitor.

In some embodiments, the fusion protein or polypeptide is administered simultaneously with the second agent or therapy. In some embodiments, the fusion protein or polypeptide is administered concurrently with the second agent or therapy. In some embodiments, the fusion protein or polypeptide is administered sequentially with the second agent or therapy. In some embodiments, the fusion protein or polypeptide is administered in the same unit dosage form as the second agent or therapy. In some embodiment, the fusion protein or polypeptide is administered in a different unit dosage form from the second agent or therapy.

### VI. Compositions, Kits and Articles of Manufacture

Also provided herein are compositions (such as formulations) comprising any one of the fusion proteins or polypeptides described herein, a nucleic acid encoding any of the fusion proteins or a portion thereof, a vector comprising the nucleic acid encoding one of the fusion proteins, or a host cell comprising the nucleic acid or vector.

Suitable formulations of the fusion proteins or polypeptides described herein can be obtained by mixing the fusion protein having the desired degree of purity with optional pharmaceutically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed. Lyophilized formulations adapted for subcutaneous administration are described in WO97/04801. Such lyophilized formulations may be reconstituted with a suitable diluent to a high protein concentration and the reconstituted formulation may be administered subcutaneously to the individual to be imaged, diagnosed, or treated herein.

The formulations to be used for in vivo administration must be sterile. This is readily accomplished by, e.g., filtration through sterile filtration membranes.

Also provided are kits comprising any one of the fusion proteins or polypeptides described herein. The kits may be useful for any of the methods of treatment described herein.

In some embodiments, the kit further comprises a device capable of delivering any of the fusion proteins or polypeptides into an individual. One type of device, for applications such as parenteral delivery, is a syringe that is used to inject the composition into the body of a subject. Inhalation devices may also be used for certain applications.

In some embodiments, the kit further comprises a therapeutic agent for treating a disease or condition, e.g., a cancer.

The kits of the present application are in suitable packaging. Suitable packaging includes, but is not limited to, vials, bottles, jars, flexible packaging (e.g., sealed Mylar or plastic bags), and the like. Kits may optionally provide additional components such as buffers and interpretative information.

The present application thus also provides articles of manufacture. The article of manufacture can comprise a container and a label or package insert on or associated with the container. Suitable containers include vials (such as sealed vials), bottles, jars, flexible packaging, and the like. Generally, the container holds a composition, and may have a sterile access port (for example the container may be an intravenous solution bag or a vial having a stopper pierceable by a hypodermic injection needle).

Those skilled in the art will recognize that several embodiments are possible within the scope and spirit of this invention. The invention will now be described in greater detail by reference to the following non-limiting examples. The following examples further illustrate the invention but, of course, should not be construed as in any way limiting its scope.

### EXEMPLARY EMBODIMENTS

Embodiment 1. A fusion protein comprising: a) a full-length antibody that specifically binds to PD-L1 and comprises two heavy chains and two light chains, wherein each of the two heavy chains comprises a heavy chain variable region (V_{H}) and each of the two light chains comprises a light chain variable region (V_{L}); b) a TGFβ superfamily receptor ectodomain; wherein the TGFβ superfamily receptor ectodomain is fused to at least one of the heavy chains or light chains of the anti-PD-L1 antibody.

Embodiment 2. The fusion protein of embodiment 1, wherein the TGFβ superfamily receptor ectodomain is fused to the at least one or both of the two light chains.

Embodiment 3. The fusion protein of embodiment 2, wherein the TGFβ superfamily receptor ectodomain is fused to the C-terminus of the at least one or both of the two light chains.

Embodiment 4. The fusion protein of embodiment 2 or embodiment 3, wherein the TGFβ superfamily receptor ectodomain is fused to the N-terminus of the at least one or both of the two light chains.

Embodiment 5. The fusion protein of any one of embodiments 1-4, wherein the TGFβ superfamily receptor ectodomain is fused to the at least one or both of the two heavy chains.

Embodiment 6. The fusion protein of embodiment 5, wherein the TGFβ superfamily receptor ectodomain is fused to the C-terminus of the at least one or both of the two heavy chains.

Embodiment 7. The fusion protein of embodiment 5 or embodiment 6, wherein the TGFβ superfamily receptor ectodomain is fused to the N-terminus of at least one or both of the two heavy chains.

Embodiment 8. The fusion protein of any one of embodiments 1-7, wherein the TGFβ superfamily receptor ectodomain comprises a TGFβ receptor type II ectodomain or a variant thereof.

Embodiment 9. The fusion protein of embodiment 8, wherein the TGFβ receptor type II ectodomain or a variant thereof comprises a human TGFβ receptor type II ectodomain or a variant thereof.

Embodiment 10. The fusion protein of embodiment 9, wherein the human TGFβ receptor type II ectodomain or a variant thereof comprises a amino acid sequence that has at least about 90% sequence identity to SEQ ID NO: 2, wherein the variant comprises one or more amino acid substitution at position N47, N71, and/or N131 according to the numbering of SEQ ID NO: 2.

Embodiment 11. The fusion protein of embodiment 10, wherein the variant comprises a N47Q mutation according to the numbering of SEQ ID NO: 2.

Embodiment 12. The fusion protein of embodiment 10 or embodiment 11, wherein the variant comprises a N71Q mutation according to the numbering of SEQ ID NO: 2.

Embodiment 13. The fusion protein of any one of embodiments 10-12, wherein the variant comprises a N131Q mutation according to the numbering of SEQ ID NO: 2.

Embodiment 14. The fusion protein of any one of embodiments 10-13, wherein the variant comprises the amino acid sequence of SEQ ID NO: 3.

Embodiment 15. The fusion protein of any one of embodiments 1-14, wherein the TGFβ superfamily receptor ectodomain comprises a TGFβ receptor type I ectodomain or a variant thereof.

Embodiment 16. The fusion protein of embodiment 15, wherein the TGFβ receptor type I ectodomain or a variant thereof comprises a human TGFβ receptor type I ectodomain or a variant thereof.

Embodiment 17. The fusion protein of any one of embodiments 1-16, wherein: a) the V_{H} comprises: i) a HC-CDR1 comprising an amino acid sequence of SEQ ID NO: 6; ii) a HC-CDR2 comprising an amino acid sequence of SEQ ID NO: 7; and iii) a HC-CDR3 comprising an amino acid sequence of SEQ ID NO: 8; and, b) the V_{L} comprises: i) a LC-CDR1 comprising an amino acid sequence of SEQ ID NO: 9; ii) a LC-CDR2 comprising an amino acid sequence of SEQ ID NO: 10; and iii) a LC-CDR3 comprising an amino acid sequence of SEQ ID NO: 11.

Embodiment 18. The fusion protein of any one of embodiments 1-17, wherein the TGFβ superfamily receptor ectodomain is fused to at least one of the heavy chains or light chains of the anti-PD-L1 antibody via a linker.

Embodiment 19. The fusion protein of embodiment 18, wherein the linker is a peptide linker.

Embodiment 20. The fusion protein of embodiment 19, wherein the peptide linker has a length of about 2 to about 25 amino acids.

Embodiment 21. The fusion protein of embodiment 20, wherein the peptide linker has a length of about 2 to about 10 amino acids.

Embodiment 22. The fusion protein of any one of embodiments 18-21, wherein the linker is a GS linker or comprises a modified sequence derived from the hinge region of an IgG.

Embodiment 23. The fusion protein of any one of embodiments 18-22, wherein the linker comprises the amino acid sequence of SEQ ID NO: 56-68.

Embodiment 24. The fusion protein of any one of embodiments 1-23, wherein the V_{H} comprises the amino acid sequence of SEQ ID NO: 12 or a variant thereof having at least about 80% sequence identity, and wherein the V_{L} comprises the amino acid sequence of SEQ ID NO: 13 or a variant thereof having at least about 80% sequence identity.

Embodiment 25. The fusion protein of any one of embodiments 1-24, wherein the heavy chain comprises the amino acid sequence of any one of SEQ ID NOs: 14-17 or a variant thereof having at least about 80% sequence identity, and wherein the light chain comprises the amino acid sequence of SEQ ID NO: 18 or a variant thereof having at least about 80% sequence identity.

Embodiment 26. The fusion protein of embodiment 25, wherein the heavy chain comprises the amino acid sequence of SEQ ID NO: 16 or 17, and wherein the light chain comprises the amino acid sequence of SEQ ID NO: 18.

Embodiment 27. The fusion protein of any one of embodiments 2-26, wherein the TGFβ superfamily receptor ectodomain or the polypeptide fused to at least one or both of the two light chains comprises the amino acid sequence of any one of SEQ ID NOs: 27-38 or a variant thereof having at least about 80% sequence identity.

Embodiment 28. The fusion protein of any one of embodiments 5-27, wherein the TGFβ superfamily receptor ectodomain fused to at least one or both of the two heavy chains comprises the amino acid sequence of any one of SEQ ID NOs: 22-26 or a variant thereof having at least about 80% sequence identity.

Embodiment 29. The fusion protein of embodiment 27 or embodiment 28, wherein: a) the TGFβ superfamily receptor ectodomain is fused to both of the two heavy chains comprising the amino acid sequence of SEQ ID NO: 22, and the two light chains comprise the amino acid sequence of SEQ ID NO: 18; b) the TGFβ superfamily receptor ectodomain is fused to both of the two heavy chains comprising the amino acid sequence of SEQ ID NO: 23, and the two light chains comprise the amino acid sequence of SEQ ID NO: 18; c) the TGFβ superfamily receptor ectodomain is fused to both of the two light chains comprising the amino acid sequence of SEQ ID NO: 27, and both of the two heavy chains comprises the amino acid sequence of SEQ ID NO: 16 or 17; d) the TGFβ superfamily receptor ectodomain is fused to both of the two light chains comprising the amino acid sequence of SEQ ID NO: 28, and both of the two heavy chains comprises the amino acid sequence of SEQ ID NO: 16 or 17; e) the TGFβ superfamily receptor ectodomain is fused to both of the two heavy chains comprising the amino acid sequence of SEQ ID NO: 22, and the TGFβ superfamily receptor ectodomain is also fused to both of the two light chains comprising the amino acid sequence of SEQ ID NO: 28; f) the TGFβ superfamily receptor ectodomain is fused to both of the two heavy chains comprising the amino acid sequence of SEQ ID NO: 23, and the TGFβ superfamily receptor ectodomain is also fused to both of the two light chains comprising the amino acid sequence of SEQ ID NO: 27; g) the TGFβ superfamily receptor ectodomain is fused to both of the two heavy chains comprising the amino acid sequence of SEQ ID NO: 22, and the TGFβ superfamily receptor ectodomain is also fused to both of the two light chains comprising the amino acid sequence of SEQ ID NO: 27; h) the TGFβ superfamily receptor ectodomain is fused to both of the two heavy chains comprising the amino acid sequence of SEQ ID NO: 23, and the TGFβ superfamily receptor ectodomain is also fused to both of the two light chains comprising the amino acid sequence of SEQ ID NO: 28; i) the polypeptide is fused to both of the two heavy chains comprising the amino acid sequence of SEQ ID NO: 24, and both of the two light chains comprise the amino acid sequence of SEQ ID NO: 18; j) the TGFβ superfamily receptor ectodomain is fused to both of the two heavy chains comprising the amino acid sequence of SEQ ID NO: 25, and both of the two light chains comprise the amino acid sequence of SEQ ID NO: 18; k) the TGFβ superfamily receptor ectodomain is fused to both of the two heavy chains comprising the amino acid sequence of SEQ ID NO: 26, and both of the two light chains comprise the amino acid sequence of SEQ ID NO: 18; l) the TGFβ superfamily receptor ectodomain is fused to both of the two light chains comprising the amino acid sequence of SEQ ID NO: 29, and both of the two heavy chains comprise the amino acid sequence of SEQ ID NO: 16 or 17; m) the TGFβ superfamily receptor ectodomain is fused to both of the two light chains comprising the amino acid sequence of SEQ ID NO: 30, and both of the two heavy chains comprise the amino acid sequence of SEQ ID NO: 16 or 17; n) the TGFβ superfamily receptor ectodomain is fused to both of the two light chains comprising the amino acid sequence of SEQ ID NO: 31, and both of the two heavy chains comprise the amino acid sequence of SEQ ID NO: 16 or 17; o) the TGFβ superfamily receptor ectodomain is fused to both of the two light chains comprising the amino acid sequence of SEQ ID NO: 32, and both of the two heavy chains comprise the amino acid sequence of SEQ ID NO: 16 or 17; p) the TGFβ superfamily receptor ectodomain is fused to both of the two light chains comprising the amino acid sequence of SEQ ID NO: 33, and both of the two heavy chains comprise the amino acid sequence of SEQ ID NO: 16 or 17; q) the TGFβ superfamily receptor ectodomain is fused to both of the two light chains comprising the amino acid sequence of SEQ ID NO: 34, and both of the two heavy chains comprise the amino acid sequence of SEQ ID NO: 16 or 17; r) the TGFβ superfamily receptor ectodomain is fused to both of the two light chains comprising the amino acid sequence of SEQ ID NO: 35 and both of the two heavy chains comprise the amino acid sequence of SEQ ID NO: 16 or 17; s) the TGFβ superfamily receptor ectodomain is fused to both of the two light chains comprising the amino acid sequence of SEQ ID NO: 36 and both of the two heavy chains comprise the amino acid sequence of SEQ ID NO: 16 or 17; or t) the TGFβ superfamily receptor ectodomain is fused to both of the two light chains comprising the amino acid sequence of SEQ ID NO: 37 and both of the two heavy chains comprise the amino acid sequence of SEQ ID NO: 16 or 17.

Embodiment 30. A polypeptide comprising a variant human TGFβ receptor type II ectodomain that has at least about 90% sequence identity to SEQ ID NO: 2, wherein the variant comprises one or more amino acid substitution at position N47, N71, and/or N131 according to the numbering of SEQ ID NO: 2.

Embodiment 31. The polypeptide of embodiment 30, wherein the variant comprises a N47Q mutation according to SEQ ID NO: 2.

Embodiment 32. The polypeptide of embodiment 30 or embodiment 31, wherein the variant comprises a N71Q mutation according to SEQ ID NO: 2.

Embodiment 33. The polypeptide of any one of embodiments 30-32, wherein the variant comprises a N131Q mutation according to SEQ ID NO: 2.

Embodiment 34. The polypeptide of any one of embodiments 30-33, wherein the variant comprises the amino acid sequence of SEQ ID NO: 3.

Embodiment 35. A fusion protein comprising a) a polypeptide of any one of embodiments 30-34; and b) a second moiety.

Embodiment 36. The fusion protein of embodiment 35, wherein the second moiety comprises a half-life extending domain.

Embodiment 37. The fusion protein of embodiment 36, wherein the second moiety comprises an Fc fragment.

Embodiment 38. The fusion protein of embodiment 37, wherein the second moiety comprises an immune checkpoint inhibitor.

Embodiment 39. The fusion protein of embodiment 38, wherein the immune checkpoint inhibitor comprises an anti-PD-L1 antibody moiety.

Embodiment 40. A pharmaceutical composition comprising the fusion protein or polypeptide of any one of embodiments 1-39.

Embodiment 41. A nucleic acid encoding the fusion protein or a portion thereof, or the polypeptide of any one of embodiments 1-39.

Embodiment 42. A nucleic acid comprising the nucleic acid sequence selected from the group consisting of SEQ ID NOs: 19-21 and 38-53.

Embodiment 43. A vector comprising the nucleic acid of embodiment 41 or embodiment 42.

Embodiment 44. A host cell comprising the nucleic acid of embodiment 41 or embodiment 42, or the vector of embodiment 43.

Embodiment 45. A method of producing the fusion protein or polypeptide of any one of embodiments 1-39, comprising: a) culturing the host cell of embodiment 44 under conditions effective to express the fusion protein or the polypeptide; and b) obtaining the expressed fusion protein or polypeptide from the host cell.

Embodiment 46. A method of treating a disease or condition in an individual, comprising administering to the individual an effective amount of the fusion protein of any one of embodiments 1-29 and 35-39, or the pharmaceutical composition of embodiment 40.

Embodiment 47. The method of embodiment 46, wherein the disease or condition is a cancer.

Embodiment 48. The method of embodiment 47, wherein the cancer is non-small cell lung cancer (NSCLC), small cell lung cancer (SCLC), cervical cancer, oropharyngeal cancer, anal cancer, vaginal or penile cancer, biliary tract cancer, cholangiocarcinoma, gallbladder cancer, head and neck cancer, pancreas cancer, prostate cancer, urothelial cancer, bladder cancer, genitourinary cancer, urogenital cancer, gastric cancer, breast cancer or colorectal cancer.

Embodiment 49. The method of embodiment 47 or embodiment 48, wherein the cancer is advanced or metastatic cancer.

Embodiment 50. The method of any one of embodiments 46-49, wherein the method further comprises administering a second agent or therapy.

Embodiment 51. The method of any one of embodiments 46-50, wherein the fusion protein or the pharmaceutical composition is administered parenterally into the individual.

Embodiment 52. The method of any one of embodiments 46-51, wherein the individual is a human.

Embodiment 53. A kit comprising the pharmaceutical composition of embodiment 40 and an instruction for treating a disease or condition.

### EXAMPLES

The examples below are intended to be purely exemplary of the application and should therefore not be considered to limit the application in any way. The following examples and detailed description are offered by way of illustration and not by way of limitation.

### Example 1. Construction and Expression of PDL1-TGFβ Receptor Fusion Protein

A series of PDL1-TGFβ receptor fusion proteins were designed by using an anti-PD-L1 monoclonal antibody (mAb), TGFβ Receptor I ectodomain (TBRI-ECD) and/or TGFβ Receptor II ectodomain (TBRII-ECD). For the construction of these fusion proteins, TBRI-ECD and/or TBRII-ECD were/was fused to the light/heavy chain of anti-PD-L1 mAb.

Particularly, exemplary TGFβ receptor ectodomain was located at the C-terminus of heavy chain or light chain of anti-PD-L1 mAb with different kinds of linkers (E-linker: EPKSSDKTHTSPPSP, E2-linker: ERKSSVESPPSP, G21-linker: GGGGSGGGGSGGGGSGGGGSG, G15-linker: (G₄S)3, G9-linker: GGSGGGGSG, G4-linker: GGGS, G2-linker: GS, E4-linker: SPPS, E5-linker: SPPSP, E5a-linker: GPPGP, E8-linker: DKTHTSPP, E10-linker: DKTHTSPPSP and E12-linker: ESKYGPPSPPSP) for fusion. Some fusion protein constructs are composed of one fusion polypeptide chain and one native polypeptide chain, other fusion protein constructs are composed of both fusion polypeptide chains. The DNA sequence expressing each polypeptide chain was inserted into pTT5 vector between EcoRI and HindIII restriction sites. Each plasmid also includes secretion signal sequence for proteins secreted into the growth medium. M7824 fusion protein was used as a reference protein. The fusion proteins are shown as below in Table 3.

TABLE 3

| Protein | Component | Plasmid |
|---|---|---|
| Anti-PD-L1 mAb | H0 | pTT5-PDLlHCv1 |
| | L0 | pTT5-ADD19-15-L |
| ADD19-15-00 | H1 | pTT5-ADD19-15-H |
| | L0 | pTT5-ADD19-15-L |
| ADD19-15-01 | H2 | pTT5-ADD19-15-H01 |
| | L0 | pTT5-ADD19-15-L |
| ADD19-15-02 | H3 | pTT5-ADD19-15-H02 |
| | L0 | pTT5-ADD19-15-L |
| ADD19-15-03 | Hl | pTT5-ADD19-15-H |
| | L1 | pTT5-ADD19-15-L03 |
| ADD19-15-04 | Hl | pTT5-ADD19-15-H |
| | L2 | pTT5-ADD19-15-L04 |
| ADD19-15-05 | H2 | pTT5-ADD19-15-H01 |
| | L2 | pTT5-ADD19-15-L04 |
| ADD19-15-06 | H3 | pTT5-ADD19-15-H02 |
| | L1 | pTT5-ADD19-15-L03 |
| ADD19-15-07 | H2 | pTT5-ADD19-15-H01 |
| | L1 | pTT5-ADD19-15-L03 |
| ADD19-15-08 | H3 | pTT5-ADD19-15-H02 |
| | L2 | pTT5-ADD19-15-L04 |
| ADD19-15-09 | H4 | pTT5-ADD19-15-H09 |
| | L0 | pTT5-ADD19-15-L |
| ADD19-15-10 | H5 | pTT5-ADD19-15-H10 |
| | L0 | pTT5-ADD19-15-L |
| ADD19-15-H01-E | H6 | pTT5-ADD19-15-H01-E |
| | L0 | pTT5-ADD19-15-L |
| ADD19-15-L03-E | H1 | pTT5-ADD19-15-H |
| | L3 | pTT5-ADD19-15-L03-E |
| ADD19-15-L03-E4 | H1 | pTT5-ADD19-15-H |
| | L4 | pTT5-ADD19-15-L03-E4 |
| ADD19-15-L03-E5 | H1 | pTT5-ADD19-15-H |
| | L5 | pTT5-ADD19-15-L03-E5 |
| ADD19-15-L03-E5a | H1 | pTT5-ADD19-15-H |
| | L6 | pTT5-ADD19-15-L03-E5a |
| ADD19-15-L03-E8 | H1 | pTT5-ADD19-15-H |
| | L7 | pTT5-ADD19-15-L03-E8 |
| ADD19-15-L03-E10 | H1 | pTT5-ADD19-15-H |
| | L8 | pTT5-ADD19-15-L03-E10 |
| ADD19-15-L03-E12 | H1 | pTT5-ADD19-15-H |
| | L9 | pTT5-ADD19-15-L03-E12 |
| ADD19-15-L03-G2 | H1 | pTT5-ADD19-15-H |
| | L10 | pTT5-ADD19-15-L03-G2 |
| ADD19-15-L03-G4 | H1 | pTT5-ADD19-15-H |
| | L11 | pTT5-ADD19-15-L03-G4 |

CHO-3E7 cells transfected with expression plasmids were cultured at 37° C. and 100 rpm for 6 days. The supernatant fraction was collected by centrifugation and the fusion protein was purified through Protein A column.

As mentioned above, TGFβ receptor ectodomain including type I ectodomain and type II ectodomain were used for fusion protein construction. Anti-PD-L1 mAb consists of heavy chain called H0 and light chain called L0. The heavy chain H0 was modified with sites mutation of K214R and A297N in Fc region generating new polypeptide called H1. Combination of new heavy chain H1 and native light chain of L0 led to a modified anti-PD-L1 mAb called ADD19-15-00, and a series of fusion proteins were designed by fusing TGFβ receptor ectodomain to ADD19-15-00 with different linkers. TBRII-ECD was fused to the C-terminus of heavy chain of H1 by G21-linker generating new polypeptide called H2. In the same way, new polypeptides called H3 were generated by using TBRI-ECD. The polypeptide H2 was engineered with sites mutation of N47Q, N71Q and N131Q within TBRII-ECD generating new polypeptide called H4. TBRI-ECD was fused to the C-terminus of H2 by G15-linker generating new polypeptide called H5. TBRII-ECD was fused to the C-terminus of heavy chain of H1 by E-linker generating new polypeptide called H6. TBRII-ECD was fused to the C-terminus of light chain of L0 by G21-linker generating new polypeptide called L1. TBRI-ECD was fused to the C-terminus of light chain of L0 by G21-linker generating new polypeptide called L2. TBRII-ECD was linked to the C-terminus of light chain of L0 by E/E4/E5/E5a/E8/E10/E12/G2/G4-linker generating nine new polypeptides called L3, L4, L5, L6, L7, L8, L9, L10 and L11, respectively.

Using this new polypeptides, a series of antibody fusion proteins were generated by combining new heavy chain fusion protein and native light chain of L0, or by combining new light chain fusion protein and native heavy chain of H1, or by combining new heavy chain polypeptide and new light chain polypeptide. Combination of new heavy chain polypeptide and native light chain of L0 led to new fusion proteins called ADD19-15-01, ADD19-15-02, ADD19-15-09, ADD19-15-10 and ADD19-15-H01-E. Combining new light chain polypeptide and native heavy chain of H1 generated new fusion proteins called ADD19-15-03, ADD19 04, ADD19-15-L03-E, ADD19-15-L03-E4, ADD19-15-L03-E5, ADD19-15-L03-E5a, ADD19-15-L03-E8, ADD19-15-L03-E10, ADD19-15-L03-E12, ADD19-15-L03-G2 and ADD19-15-L03-G4. Combining new heavy chain polypeptide and new light chain polypeptide generated new fusion proteins called ADD19-15-05 and ADD19-15-08.

### Example 2. Characterization of the Fusion Proteins

### A. ELISA Binding Assay

The 96-well disposable microwells were coated with TGF-β1, TGF-β2 or TGF-β3 for 16 hours at 4° C. After washing by washing buffer, the coated plates were blocked by blocking buffer for 2 hours at 37° C. The plates were then incubated with diluted samples at 37° C. for 1 hour followed by HRP-conjugated 2nd antibody incubation at 37° C. for 0.5 hour. After washing and TMB substrate incubation, stop solution was added into each well before reading by plate reader with 450 nM.

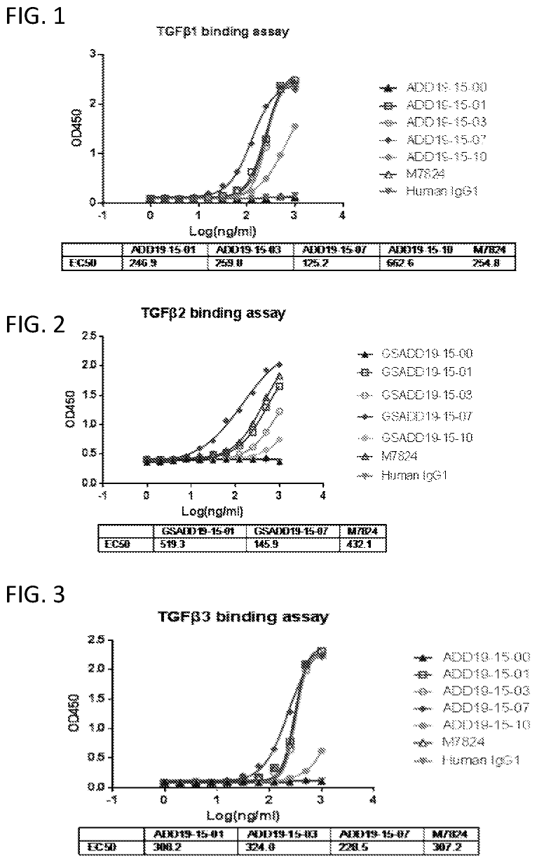

Among the first batch of fusion proteins as shown in FIGS. 1-3, compared with benchmark of M7824, (1) ADD19-15-01 showed similar binding affinity to TGFβ1, TGFβ2 and TGFβ3; (2) ADD19-15-03 showed similar TGFβ1 and TGFβ3 binding affinity but lower TGFβ2 binding affinity; (3) ADD19-15-07 showed higher binding affinity to all of three TGFβ proteins; (4) ADD19-15-10 showed lower binding affinity to all of three TGFβ proteins.

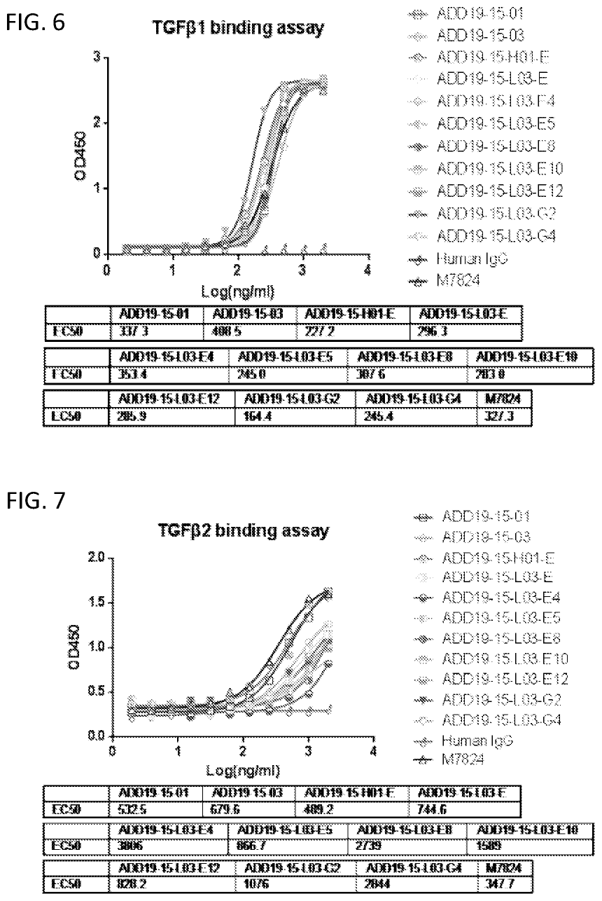

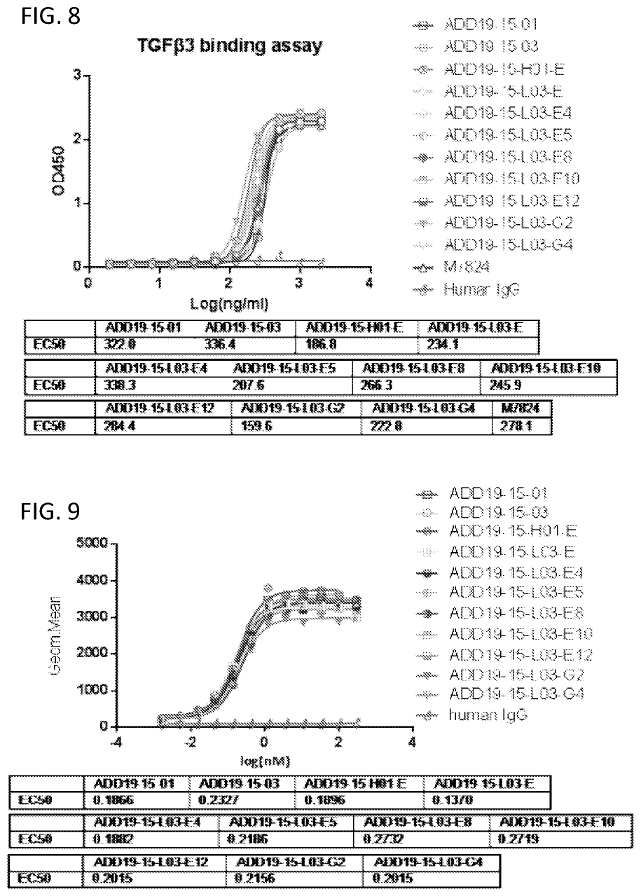

Among the second batch of fusion proteins as shown in FIGS. 6-8, compared with benchmark of M7824, all fusion proteins showed similar TGFβ1 and TGFβ3 binding affinity but diverse TGFβ2 binding affinity. ADD19-15-03 has been identified as a lower TGFβ2 binder in the first batch of fusion proteins, some fusion proteins in the second batch showed even lower TGFβ2 binding affinity including ADD19-15-03-E4, ADD19-15-03-E5, ADD19-15-03-E8, ADD19-15-03-E10, ADD19-15-03-E12, ADD19-15-03-G2 and ADD19-15-03-G4.

### B. SPR Affinity Assay

TGFβ ligands are covalently immobilized onto the CMS sensor chip via amine coupling chemistry. The assay was performed at 25° C. and the running buffer was HBS-EP. A series of concentrations of analytes were injected over the ligand surface consecutively as association phase, followed by injecting running buffer as dissociation phase. The affinity and kinetics of TGFβ ligands to anti-PD-L1 mAb-TGFβR fusion proteins was summarized in Table 4.

TABLE 4
Affinity of TGF-β1, TGF-β2 and TGF-β3 to samples by SPR

| Ligand | Analyte | kₐ(1/Ms) | k_{d}(1/s) | K_{D} (M) | Rmax | Chi² (RU²) |
|---|---|---|---|---|---|---|
| TGF-β1 | GSADD19-15-00 | NA | NA | NA | NA | NA |
| TGF-β1 | M7824 | 3.34E+05 | 3.15E−04 | 9.43E−10 | 91.7 | 1.22E+00 |
| TGF-β1 | GSADD19-15-01 | 3.38E+05 | 3.25E−04 | 9.62E−10 | 96.9 | 1.32E+00 |
| TGF-β1 | GSADD19-15-03 | 3.24E+05 | 4.20E−04 | 1.30E−09 | 85 | 8.97E−01 |
| TGF-β1 | GSADD19-15-07 | 2.99E+05 | 6.77E−05 | 2.26E−10 | 58.5 | 4.79E−01 |
| TGF-β1 | GSADD19-15-10 | 2.00E+05 | 1.34E−03 | 6.73E−09 | 17.8 | 1.12E−01 |
| TGF-β2 | GSADD19-15-00 | NA | NA | NA | NA | NA |
| TGF-β2 | M7824 | 2.86E+05 | 1.32E−03 | 4.60E−09 | 47.3 | 1.99E+00 |
| TGF-β2 | GSADD19-15-01 | 3.66E+05 | 3.13E−03 | 8.55E−09 | 23 | 1.60E+00 |
| TGF-β2 | GSADD19-15-03 | 2.39E+05 | 4.11E−03 | 1.72E−08 | 21.3 | 5.10E−01 |
| TGF-β2 | GSADD19-15-07 | 2.80E+05 | 2.28E−03 | 8.14E−09 | 13.7 | 1.15E−01 |
| TGF-β2 | GSADD19-15-10 | NA | NA | NA | NA | NA |
| TGF-β3 | GSADD19-15-00 | NA | NA | NA | NA | NA |
| TGF-β3 | M7824 | 3.14E+05 | 3.20E−04 | 1.02E−09 | 32.8 | 1.44E−01 |
| TGF-β3 | GSADD19-15-01 | 3.08E+05 | 3.10E−04 | 1.01E−09 | 33.6 | 1.35E−01 |
| TGF-β3 | GSADD19-15-03 | 2.93E+05 | 3.85E−04 | 1.31E−09 | 31.5 | 1.17E−01 |
| TGF-β3 | GSADD19-15-07 | 3.10E+05 | 1.68E−04 | 5.42E−10 | 23.9 | 9.31E−02 |
| TGF-β3 | GSADD19-15-10 | 2.06E+05 | 1.07E−03 | 5.19E−09 | 6.5 | 1.40E−02 |

For TGFβ binding assay, compared with benchmark of M7824, (1) ADD19-15-01 showed similar binding affinity to TGFβ1, TGFβ2 and TGFβ3; (2) ADD19-15-03 showed similar TGFβ1 and TGFβ3 binding affinity but lower TGFβ2 binding affinity; (3) ADD19-15-07 showed similar TGFβ2 binding affinity but higher TGFβ1 and TGFβ3 binding affinity; (4) ADD19-15-10 showed lower binding affinity to all of three TGFβ proteins.

### C. FACS Binding Assay

The binding pattern of antibody fusion proteins on PD-L1 that is over expressed on CHO-K1 cells were plotted with samples in 3× serial dilutions, starting concentration of 300 nM. Antibody-antigen binding curves were generated with geometric mean values. Raw data was plotted with GraphPad Prism v6.02 software with four parameters, best-fit values program to analyze the EC50.

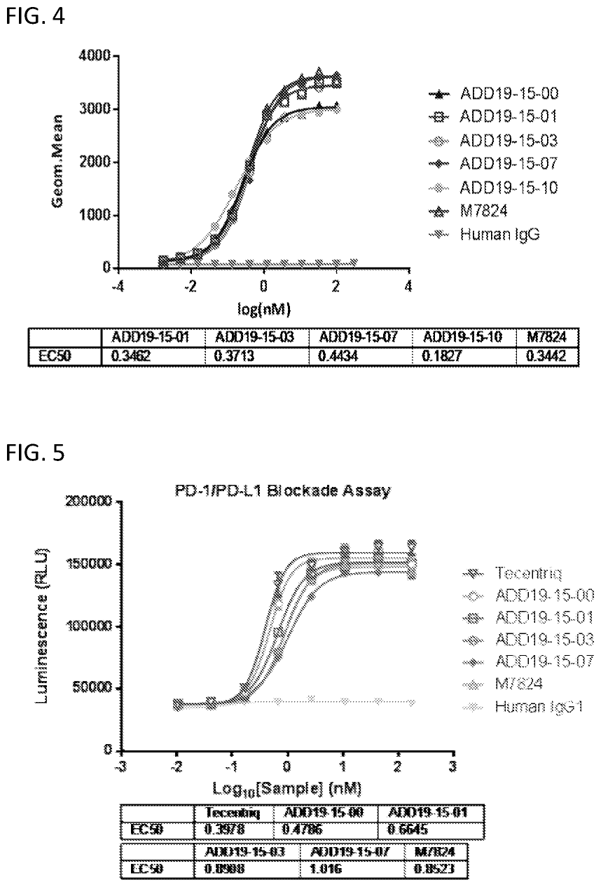

For PD-L1 binding, as shown in FIG. 4, all of antibody fusion proteins showed similar affinity to PD-L1 antigen compared to the parental mAb of ADD19-15-00. So, protein fusion did not affect antigen affinity of the final constructs. These constructs like ADD19-15-01 and ADD19-15-03 also exhibited almost the same PD-L1 binding affinity as benchmark of M7824. Meanwhile, fusion proteins with different linkers did not influence PD-L1 binding affinity as shown in FIG. 9.

### D. In Vitro Bioassay

For the in vitro bioassay of anti-PD-L1 antibody fusion proteins, PD-1/PD-L1 blockade bioassay was performed using Promega detection kit.

The PD-1/PD-L1 blockade bioassay system from Promega can be used to measure the potency and stability of antibodies and other biologics designed to block the PD-1/PD-L1 interaction. The assay consists of two genetically engineered cell lines: PD-1 effector cells, which are Jurkat T cells expressing human PD-1 and a luciferase reporter driven by an NFAT response element (NFAT-RE), and PD-L1 aAPC/CHO-K1 Cells, which are CHO-K1 cells expressing human PD-L1 and an engineered cell surface protein designed to activate cognate TCRs in an antigen-independent manner. When the two cell types are co-cultured, the PD-1/PD-L1 interaction inhibits TCR signaling and NFAT-RE-mediated luminescence. Addition of either an anti-PD-1 or anti-PD-L1 antibody that blocks the PD-1/PD-L1 interaction releases the inhibitory signal and results in TCR activation and NFAT-RE-mediated luminescence.

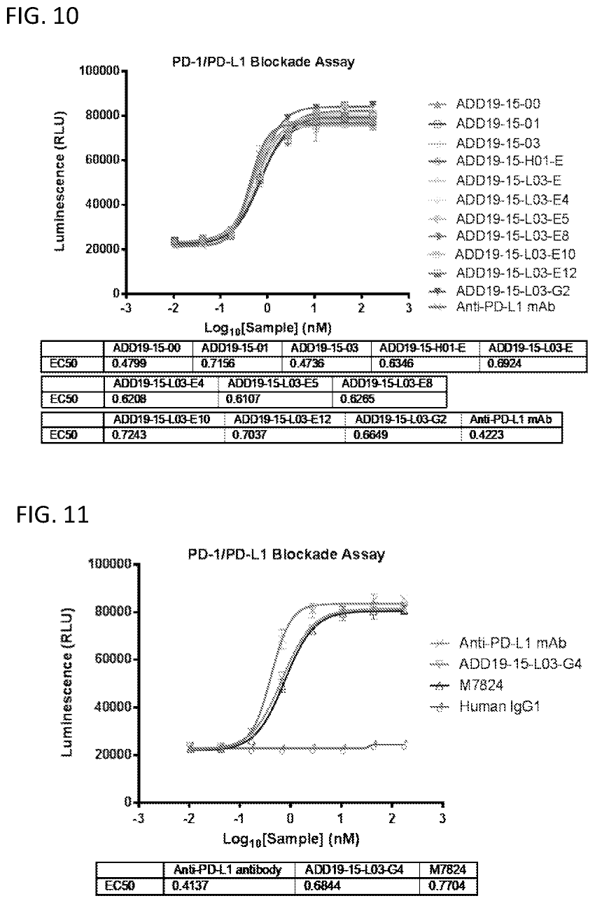

For PD-1/PD-L1 blockade bioassay, Tecentriq biosimilar was utilized as a reference antibody. As shown in FIG. 5, compared with parental mAb of ADD19-15-00, fusion proteins of ADD19-15-01 and ADD19-15-03 showed comparable activity. In addition, their efficacy are similar to that of benchmark of M7824. Meanwhile, fusion proteins with different linkers did not affect their activity as shown in FIG. 10 and FIG. 11.

SEQUENCE TABLE

| | | |
|---|---|---|
| SEQ | | |
| ID NO | Description | Nucleotide or Amino acid sequences |

Exemplary TGFβ receptor ectodomain sequences

| | | |
|---|---|---|
| 1 | TGFβR1 | QCFCHLCTKDNFTCVTDGLCFVSVTETTDKVIHNSMCIAEIDLIPRD |
| | | RPFVCAPSSKTGSVTTTYCCNQDHCNKIELPTTVKSSPGLGPVE |
| 2 | TGFβR2 | IPPHVQKSVNNDMIVTDNNGAVKFPQLCKFCDVRFSTCDNQKSCM |
| | | SNCSITSICEKPQEVCVAVWRKNDENITLETVCHDPKLPYHDFILED |
| | | AASPKCIMKEKKKPGETFFMCSCSSDECNDNIIFSEEYNTSNPD |
| 3 | TGFβR2 | IPPHVQKSVNNDMIVTDNNGAVKFPQLCKFCDVRFSTCDNQKSCM |
| | with N47Q, | SQCSITSICEKPQEVCVAVWRKNDEQITLETVCHDPKLPYHDFILED |
| | N71Q and | AASPKCIMKEKKKPGETFFMCSCSSDECNDNIIFSEEYQTSNPD |
| | N131Q | |
| | mutations | |
| 4 | TGFβR1 | cagtgcttctgccatctgtgcaccaaggacaacttcacctgcgtgaccgatggcctgtgttttgtgtccgtg |
| | | accgagacaaccgacaaagtgatccacaactccatgtgcatcgccgagatcgacctgatccctagagat |
| | | cggcccttcgtgtgtgctcctagctctaagaccggctctgtcaccacaacctactgctgtaatcaggacca |
| | | ctgcaacaagatcgagctgcctaccaccgtgaagtcctctccaggactgggccctgtggaa |
| 5 | TGFβR2 | attcctcctcacgtgcagaaatccgtgaacaacgacatgatcgtgaccgacaacaacggcgctgtgaagt |
| | | ttccacagctgtgcaagttctgcgacgtcagattctctacctgtgacaaccagaagtcctgtatgtccaactg |
| | | ctccatcacctctatctgcgagaagccccaagaggtgtgcgtggccgtgtggcggaagaacgatgagaa |
| | | catcaccctggaaaccgtgtgtcatgatcctaagctgccttaccacgacttcatcctggaagatgccgcttc |
| | | tcccaagtgcatcatgaaagagaagaagaagcctggcgagacattcttcatgtgctcttgtagctccgacg |
| | | agtgcaacgacaatatcatcttctccgaggaatacaacaccagcaatcctgac |

Exemplary anti-PDL1 monoclonal antibody

| | | |
|---|---|---|
| 6 | HC-CDR1 | GYGIT |
| 7 | HC-CDR2 | EIFPRRVQTYYSEKFKG |
| 8 | HC-CDR3 | DYDPYFALDY |
| 9 | LC-CDR1 | RASQDVSTAVD |
| 10 | LC-CDR2 | SASYRYT |
| 11 | LC-CDR3 | QQHYSIPFT |
| 12 | V_{H} | EVQLVQSGAEVKKPGASVKVSCKASGYIFTGYGITWVRQAPGQG |
| | | LEWMGEIFPRRVQTYYSEKFKGRVTMTTDTSTSTAYMELRSLRSD |
| | | DTAVYYCARDYDPYFALDYWGQGTTVTVSS |
| 13 | v_{L} | DIQMTQSPSSLSASVGDRVTITCRASQDVSTAVDWYQQKPGKAPK |
| | | LLIYSASYRYTGVPDRFSGSGSGTDFTFTISSLQPEDIATYYCQQHY |
| | | SIPFTFGQGTKLEIKRTV |
| 14 | Heavy chain | EVQLVQSGAEVKKPGASVKVSCKASGYIFTGYGITWVRQAPGQG |
| | (H0) | LEWMGEIFPRRVQTYYSEKFKGRVTMTTDTSTSTAYMELRSLRSD |
| | | DTAVYYCARDYDPYFALDYWGQGTTVTVSSASTKGPSVFPLAPSS |
| | | KSTSGGTAALGCLVKDYFPEPVTVSWNSGALTSGVHTFPAVLQSS |
| | | GLYSLSSWTVPSSSLGTQTYICNVNHKPSNTKVDKKVEPKSCDKT |
| | | HTCPPCPAPELLGGPSVFLFPPKPKDTLMISRTPEVTCVVVDVSHED |
| | | PEVKFNWYVDGVEVHNAKTKPREEQYASTYRVVSVLTVLHQDW |
| | | LNGKEYKCKVSNKALPAPIEKTISKAKGQPREPQVYTLPPSREEMT |
| | | KNQVSLTCLVKGFYPSDIAVEWESNGQPENNYKTTPPVLDSDGSF |
| | | FLYSKLTVDKSRWQQGNVFSCSVMHEALHNHYTQKSLSLSPGK |
| 15 | Heavy chain | EVQLVQSGAEVKKPGASVKVSCKASGYIFTGYGITWVRQAPGQG |
| | (H0)-with | LEWMGEIFPRRVQTYYSEKFKGRVTMTTDTSTSTAYMELRSLRSD |
| | K→A | DTAVYYCARDYDPYFALDYWGQGTTVTVSSASTKGPSVFPLAPSS |
| | mutation | KSTSGGTAALGCLVKDYFPEPVTVSWNSGALTSGVHTFPAVLQSS |
| | | GLYSLSSWTVPSSSLGTQTYICNVNHKPSNTKVDKKVEPKSCDKT |
| | | HTCPPCPAPELLGGPSVFLFPPKPKDTLMISRTPEVTCVVVDVSHED |
| | | PEVKFNWYVDGVEVHNAKTKPREEQYASTYRVVSVLTVLHQDW |
| | | LNGKEYKCKVSNKALPAPIEKTISKAKGQPREPQVYTLPPSREEMT |
| | | KNQVSLTCLVKGFYPSDIAVEWESNGQPENNYKTTPPVLDSDGSF |
| | | FLYSKLTVDKSRWQQGNVFSCSVMHEALHNHYTQKSLSLSPGA |
| 16 | Heavy chain | EVQLVQSGAEVKKPGASVKVSCKASGYIFTGYGITWVRQAPGQG |
| | (H1, with | LEWMGEIFPRRVQTYYSEKFKGRVTMTTDTSTSTAYMELRSLRSD |
| | K214R and | DTAVYYCARDYDPYFALDYWGQGTTVTVSSASTKGPSVFPLAPSS |
| | A297N | KSTSGGTAALGCLVKDYFPEPVTVSWNSGALTSGVHTFPAVLQSS |
| | mutations) | GLYSLSSWTVPSSSLGTQTYICNVNHKPSNTKVDKKVEPKSCDKT |
| | | HTCPPCPAPELLGGPSVFLFPPKPKDTLMISRTPEVTCVVVDVSHED |
| | | PEVKFNWYVDGVEVHNAKTKPREEQYNSTYRVVSVLTVLHQDW |
| | | LNGKEYKCKVSNKALPAPIEKTISKAKGQPREPQVYTLPPSREEMT |
| | | KNQVSLTCLVKGFYPSDIAVEWESNGQPENNYKTTPPVLDSDGSF |
| | | FLYSKLTVDKSRWQQGNVFSCSVMHEALHNHYTQKSLSLSPGK |
| 17 | Heavy chain | EVQLVQSGAEVKKPGASVKVSCKASGYIFTGYGITWVRQAPGQG |
| | (H1, with | LEWMGEIFPRRVQTYYSEKFKGRVTMTTDTSTSTAYMELRSLRSD |
| | K214R and | DTAVYYCARDYDPYFALDYWGQGTTVTVSSASTKGPSVFPLAPSS |
| | A297N | KSTSGGTAALGCLVKDYFPEPVTVSWNSGALTSGVHTFPAVLQSS |
| | mutations)- | GLYSLSSWTVPSSSLGTQTYICNVNHKPSNTKVDKKVEPKSCDKT |
| | with K→A | HTCPPCPAPELLGGPSVFLFPPKPKDTLMISRTPEVTCVVVDVSHED |
| | mutation | PEVKFNWYVDGVEVHNAKTKPREEQYNSTYRVVSVLTVLHQDW |
| | | LNGKEYKCKVSNKALPAPIEKTISKAKGQPREPQVYTLPPSREEMT |
| | | KNQVSLTCLVKGFYPSDIAVEWESNGQPENNYKTTPPVLDSDGSFF |
| | | LYSKLTVDKSRWQQGNVFSCSVMHEALHNHYTQKSLSLSPGA |
| 18 | Light chain | DIQMTQSPSSLSASVGDRVTITCRASQDVSTAVDWYQQKPGKAPK |
| | (L0) | LLIYSASYRYTGVPDRFSGSGSGTDFTFTISSLQPEDIATYYCQQHY |
| | | SIPFTFGQGTKLEIKRTVAAPSVFIFPPSDEQLKSGTASVVCLLNNFY |
| | | PREAKVQWKVDNALQSGNSQESVTEQDSKDSTYSLSSTLTLSKAD |
| | | YEKHKVYACEVTHQGLSSPVTKSFNRGEC |
| 19 | H0 | gaagtccagctggtgcagagcggagccgaggtgaagaaaccaggcgcttctgtgaaggtgtcctgcaa |
| | | ggcctctgggtacatcttcaccggctacggcatcacctgggtgcggcaggctcctggccagggcctgga |
| | | atggatgggcgagatctttcccaggagagtgcagacctactactccgagaagttcaagggcagagtgac |
| | | catgaccaccgacacctccacctctaccgcctacatggaactgcggtctctgagatccgatgacaccgct |
| | | gtgtactactgcgccagagactacgacccttatttcgccctggattattggggccaaggcaccaccgtgac |
| | | agtctcctccgcctctaccaagggcccttccgtgttccccctggcccctagcagcaagtccacatcagga |
| | | ggcaccgctgctctgggctgcctggtcaaggactacttccctgaacctgtgaccgtgtcctggaactccg |
| | | gcgccctgacaagtggagtgcataccttccccgccgtgctgcagtcctctggcctgtactctctgtctagc |
| | | gtggtcactgtgccttcctctagcctcggcacacagacatacatctgcaacgtgaaccacaagccttccaa |
| | | caccaaagtggacaagaaggtggaacccaagtcttgcgacaaaacccacacatgtccaccttgtcctgc |
| | | ccccgagctgctgggcggcccctccgtgtttctgtttcctcctaagccgaaggatacactgatgatctccc |
| | | ggacccctgaggtgacctgtgtggtggtggacgtgtctcacgaggaccccgaagtgaagttcaactggt |
| | | acgtggatggcgtggaagtgcacaatgctaagaccaagcctagagaagagcagtacgcctccacctac |
| | | cgggtggtctctgtgctgaccgtcctgcatcaggactggctgaacggcaaagagtacaagtgcaaggtgt |
| | | ctaacaaggctctgcctgctcctatcgagaaaaccatctctaaggccaagggacagcctcgggaaccac |
| | | aagtgtacaccctgcctccttctagagaggagatgaccaagaaccaggtgagcctgacctgcctcgtgaa |
| | | aggcttctacccctctgacatcgccgtggagtgggagtccaatggccagcctgagaacaactacaagac |
| | | cacacctccagttctggactccgacggttccttcttcctgtactccaagctgaccgttgataagtccagatgg |
| | | cagcagggcaacgtgttctcctgttccgtgatgcacgaggccctgcacaaccactacacccagaagtcc |
| | | ctgagcttgtctcctggcaag |
| 20 | H1 | gaagtccagctggtgcagagcggagccgaggtgaagaaaccaggcgcttctgtgaaggtgtcctgcaa |
| | | ggcctctgggtacatcttcaccggctacggcatcacctgggtgcggcaggctcctggccagggcctgga |
| | | atggatgggcgagatctttcccaggagagtgcagacctactactccgagaagttcaagggcagagtgac |
| | | catgaccaccgacacctccacctctaccgcctacatggaactgcggtctctgagatccgatgacaccgct |
| | | gtgtactactgcgccagagactacgacccttatttcgccctggattattggggccaaggcaccaccgtgac |
| | | agtctcctccgcctctaccaagggcccttccgtgttccccctggcccctagcagcaagtccacatcagga |
| | | ggcaccgctgctctgggctgcctggtcaaggactacttccctgaacctgtgaccgtgtcctggaactccg |
| | | gcgccctgacaagtggagtgcataccttccccgccgtgctgcagtcctctggcctgtactctctgtctagc |
| | | gtggtcactgtgccttcctctagcctcggcacacagacatacatctgcaacgtgaaccacaagccttccaa |
| | | caccaaagtggacaagagagtggaacccaagtcttgcgacaaaacccacacatgtccaccttgtcctgc |
| | | ccccgagctgctgggcggcccctccgtgtttctgtttcctcctaagccgaaggatacactgatgatctccc |
| | | ggacccctgaggtgacctgtgtggtggtggacgtgtctcacgaggaccccgaagtgaagttcaactggt |
| | | acgtggatggcgtggaagtgcacaatgctaagaccaagcctagagaagagcagtacaactccacctac |
| | | cgggtggtctctgtgctgaccgtcctgcatcaggactggctgaacggcaaagagtacaagtgcaaggtgt |
| | | ctaacaaggctctgcctgctcctatcgagaaaaccatctctaaggccaagggacagcctcgggaaccac |
| | | aagtgtacaccctgcctccttctagagaggagatgaccaagaaccaggtgagcctgacctgcctcgtgaa |
| | | aggcttctacccctctgacatcgccgtggagtgggagtccaatggccagcctgagaacaactacaagac |
| | | cacacctccagttctggactccgacggttccttcttcctgtactccaagctgaccgttgataagtccagatgg |
| | | cagcagggcaacgtgttctcctgttccgtgatgcacgaggccctgcacaaccactacacccagaagtcc |
| | | ctgagcttgtctcctggcaag |
| 21 | L0 | gatatccagatgacccagtctcctagcagcctgagcgcttctgtgggcgacagagtgacaatcacctgta |
| | | gagcctctcaggacgtgtccaccgccgtggattggtaccagcagaagcccggcaaggctcctaagctg |
| | | ctgatctactctgcctcctaccggtacacaggagtccccgatagattctctggctccggctctggaaccga |
| | | cttcaccttcaccatctcctctctgcagcctgaggacattgccacctactactgccagcagcactactccatc |
| | | ccttttaccttcggccagggcaccaagctggaaatcaagcggaccgtggccgctccatccgtgttcatcttt |
| | | cctccttccgacgagcagctgaagtctggcaccgcttccgtggtgtgcctgctgaacaacttctaccctcg |
| | | ggaagccaaggtgcagtggaaagtggacaacgccctgcagtccggcaatagccaagagtccgtcacc |
| | | gagcaagactccaaggactctacctattctctctccagcacactgaccctgtctaaagccgactacgagaa |
| | | gcacaaggtgtacgcctgcgaagtgacccaccagggcctgtcttcccccgtgacaaagtccttcaacag |
| | | aggcgagtgt |

Exemplary fusion protein sequences

| | | |
|---|---|---|
| 22 | H2 (anti-PD- | EVQLVQSGAEVKKPGASVKVSCKASGYIFTGYGITWVRQAPGQG |
| | L1-TGFβR2) | LEWMGEIFPRRVQTYYSEKFKGRVTMTTDTSTSTAYMELRSLRSD |
| | | DTAVYYCARDYDPYFALDYWGQGTTVTVSSASTKGPSVFPLAPSS |
| | | KSTSGGTAALGCLVKDYFPEPVTVSWNSGALTSGVHTFPAVLQSS |
| | | GLYSLSSWTVPSSSLGTQTYICNVNHKPSNTKVDKRVEPKSCDKT |
| | | HTCPPCPAPELLGGPSVFLFPPKPKDTLMISRTPEVTCVVVDVSHED |
| | | PEVKFNWYVDGVEVHNAKTKPREEQYNSTYRVVSVLTVLHQDW |
| | | LNGKEYKCKVSNKALPAPIEKTISKAKGQPREPQVYTLPPSREEMT |
| | | KNQVSLTCLVKGFYPSDIAVEWESNGQPENNYKTTPPVLDSDGSF |
| | | FLYSKLTVDKSRWQQGNVFSCSVMHEALHNHYTQKSLSLSPGAG |
| | | GGGSGGGGSGGGGSGGGGSGIPPHVQKSVNNDMIVTDNNGAVKF |
| | | PQLCKFCDVRFSTCDNQKSCMSNCSITSICEKPQEVCVAVWRKND |
| | | ENITLETVCHDPKLPYHDFILEDAASPKCIMKEKKKPGETFFMCSC |
| | | SSDECNDNIIFSEEYNTSNPD |
| 23 | H3 | EVQLVQSGAEVKKPGASVKVSCKASGYIFTGYGITWVRQAPGQG |
| | | LEWMGEIFPRRVQTYYSEKFKGRVTMTTDTSTSTAYMELRSLRSD |
| | | DTAVYYCARDYDPYFALDYWGQGTTVTVSSASTKGPSVFPLAPSS |
| | | KSTSGGTAALGCLVKDYFPEPVTVSWNSGALTSGVHTFPAVLQSS |
| | | GLYSLSSWTVPSSSLGTQTYICNVNHKPSNTKVDKRVEPKSCDKT |
| | | HTCPPCPAPELLGGPSVFLFPPKPKDTLMISRTPEVTCVVVDVSHED |
| | | PEVKFNWYVDGVEVHNAKTKPREEQYNSTYRVVSVLTVLHQDW |
| | | LNGKEYKCKVSNKALPAPIEKTISKAKGQPREPQVYTLPPSREEMT |
| | | KNQVSLTCLVKGFYPSDIAVEWESNGQPENNYKTTPPVLDSDGSF |
| | | FLYSKLTVDKSRWQQGNVFSCSVMHEALHNHYTQKSLSLSPGAG |
| | | GGGSGGGGSGGGGSGGGGSGQCFCHLCTKDNFTCVTDGLCFVSV |
| | | TETTDKVIHNSMCIAEIDLIPRDRPFVCAPSSKTGSVTTTYCCNQDH |
| | | CNKIELPTTVKSSPGLGPVE |
| 24 | H4 | EVQLVQSGAEVKKPGASVKVSCKASGYIFTGYGITWVRQAPGQG |
| | | LEWMGEIFPRRVQTYYSEKFKGRVTMTTDTSTSTAYMELRSLRSD |
| | | DTAVYYCARDYDPYFALDYWGQGTTVTVSSASTKGPSVFPLAPSS |
| | | KSTSGGTAALGCLVKDYFPEPVTVSWNSGALTSGVHTFPAVLQSS |
| | | GLYSLSSWTVPSSSLGTQTYICNVNHKPSNTKVDKRVEPKSCDKT |
| | | HTCPPCPAPELLGGPSVFLFPPKPKDTLMISRTPEVTCVVVDVSHED |
| | | PEVKFNWYVDGVEVHNAKTKPREEQYNSTYRVVSVLTVLHQDW |
| | | LNGKEYKCKVSNKALPAPIEKTISKAKGQPREPQVYTLPPSREEMT |
| | | KNQVSLTCLVKGFYPSDIAVEWESNGQPENNYKTTPPVLDSDGSF |
| | | FLYSKLTVDKSRWQQGNVFSCSVMHEALHNHYTQKSLSLSPGAG |
| | | GGGSGGGGSGGGGSGGGGSGIPPHVQKSVNNDMIVTDNNGAVKF |
| | | PQLCKFCDVRFSTCDNQKSCMSQCSITSICEKPQEVCVAVWRKND |
| | | EQITLETVCHDPKLPYHDFILEDAASPKCIMKEKKKPGETFFMCSC |
| | | SSDECNDNIIFSEEYQTSNPD |
| 25 | H5 | EVQLVQSGAEVKKPGASVKVSCKASGYIFTGYGITWVRQAPGQG |
| | | LEWMGEIFPRRVQTYYSEKFKGRVTMTTDTSTSTAYMELRSLRSD |
| | | DTAVYYCARDYDPYFALDYWGQGTTVTVSSASTKGPSVFPLAPSS |
| | | KSTSGGTAALGCLVKDYFPEPVTVSWNSGALTSGVHTFPAVLQSS |
| | | GLYSLSSWTVPSSSLGTQTYICNVNHKPSNTKVDKRVEPKSCDKT |
| | | HTCPPCPAPELLGGPSVFLFPPKPKDTLMISRTPEVTCVVVDVSHED |
| | | PEVKFNWYVDGVEVHNAKTKPREEQYNSTYRVVSVLTVLHQDW |
| | | LNGKEYKCKVSNKALPAPIEKTISKAKGQPREPQVYTLPPSREEMT |
| | | KNQVSLTCLVKGFYPSDIAVEWESNGQPENNYKTTPPVLDSDGSF |
| | | FLYSKLTVDKSRWQQGNVFSCSVMHEALHNHYTQKSLSLSPGAG |
| | | GGGSGGGGSGGGGSGGGGSGIPPHVQKSVNNDMIVTDNNGAVKF |
| | | PQLCKFCDVRFSTCDNQKSCMSNCSITSICEKPQEVCVAVWRKND |
| | | ENITLETVCHDPKLPYHDFILEDAASPKCIMKEKKKPGETFFMCSC |
| | | SSDECNDNIIFSEEYNTSNPDGGGGSGGGGSGGGGSQCFCHLCTKD |
| | | NFTCVTDGLCFVSVTETTDKVIHNSMCIAEIDLIPRDRPFVCAPSSK |
| | | TGSVTTTYCCNQDHCNKIELPTTVKSSPGLGPVE |
| 26 | H6 | EVQLVQSGAEVKKPGASVKVSCKASGYIFTGYGITWVRQAPGQG |
| | | LEWMGEIFPRRVQTYYSEKFKGRVTMTTDTSTSTAYMELRSLRSD |
| | | DTAVYYCARDYDPYFALDYWGQGTTVTVSSASTKGPSVFPLAPSS |
| | | KSTSGGTAALGCLVKDYFPEPVTVSWNSGALTSGVHTFPAVLQSS |
| | | GLYSLSSWTVPSSSLGTQTYICNVNHKPSNTKVDKRVEPKSCDKT |
| | | HTCPPCPAPELLGGPSVFLFPPKPKDTLMISRTPEVTCVVVDVSHED |
| | | PEVKFNWYVDGVEVHNAKTKPREEQYNSTYRVVSVLTVLHQDW |
| | | LNGKEYKCKVSNKALPAPIEKTISKAKGQPREPQVYTLPPSREEMT |
| | | KNQVSLTCLVKGFYPSDIAVEWESNGQPENNYKTTPPVLDSDGSF |
| | | FLYSKLTVDKSRWQQGNVFSCSVMHEALHNHYTQKSLSLSPGKEP |
| | | KSSDKTHTSPPSPIPPHVQKSVNNDMIVTDNNGAVKFPQLCKFCDV |
| | | RFSTCDNQKSCMSNCSITSICEKPQEVCVAVWRKNDENITLETVCH |
| | | DPKLPYHDFILEDAASPKCIMKEKKKPGETFFMCSCSSDECNDNIIF |
| | | SEEYNTSNPD |
| 27 | L1 | DIQMTQSPSSLSASVGDRVTITCRASQDVSTAVDWYQQKPGKAPK |
| | | LLIYSASYRYTGVPDRFSGSGSGTDFTFTISSLQPEDIATYYCQQHY |
| | | SIPFTFGQGTKLEIKRTVAAPSVFIFPPSDEQLKSGTASVVCLLNNFY |
| | | PREAKVQWKVDNALQSGNSQESVTEQDSKDSTYSLSSTLTLSKAD |
| | | YEKHKVYACEVTHQGLSSPVTKSFNRGECGGGGSGGGGSGGGGS |
| | | GGGGSGIPPHVQKSVNNDMIVTDNNGAVKFPQLCKFCDVRFSTCD |
| | | NQKSCMSNCSITSICEKPQEVCVAVWRKNDENITLETVCHDPKLPY |
| | | HDFILEDAASPKCIMKEKKKPGETFFMCSCSSDECNDNIIFSEEYNT |
| | | SNPD |
| 28 | L2 | DIQMTQSPSSLSASVGDRVTITCRASQDVSTAVDWYQQKPGKAPK |
| | | LLIYSASYRYTGVPDRFSGSGSGTDFTFTISSLQPEDIATYYCQQHY |
| | | SIPFTFGQGTKLEIKRTVAAPSVFIFPPSDEQLKSGTASVVCLLNNFY |
| | | PREAKVQWKVDNALQSGNSQESVTEQDSKDSTYSLSSTLTLSKAD |
| | | YEKHKVYACEVTHQGLSSPVTKSFNRGECGGGGSGGGGSGGGGS |
| | | GGGGSGQCFCHLCTKDNFTCVTDGLCFVSVTETTDKVIHNSMCIA |
| | | EIDLIPRDRPFVCAPSSKTGSVTTTYCCNQDHCNKIELPTTVKSSPG |
| | | LGPVE |
| 29 | L3 | DIQMTQSPSSLSASVGDRVTITCRASQDVSTAVDWYQQKPGKAPK |
| | | LLIYSASYRYTGVPDRFSGSGSGTDFTFTISSLQPEDIATYYCQQHY |
| | | SIPFTFGQGTKLEIKRTVAAPSVFIFPPSDEQLKSGTASVVCLLNNFY |
| | | PREAKVQWKVDNALQSGNSQESVTEQDSKDSTYSLSSTLTLSKAD |
| | | YEKHKVYACEVTHQGLSSPVTKSFNRGECEPKSSDKTHTSPPSPIPP |
| | | HVQKSVNNDMIVTDNNGAVKFPQLCKFCDVRFSTCDNQKSCMSN |
| | | CSITSICEKPQEVCVAVWRKNDENITLETVCHDPKLPYHDFILEDA |
| | | ASPKCIMKEKKKPGETFFMCSCSSDECNDNIIFSEEYNTSNPD |
| 30 | L4 | DIQMTQSPSSLSASVGDRVTITCRASQDVSTAVDWYQQKPGKAPK |
| | | LLIYSASYRYTGVPDRFSGSGSGTDFTFTISSLQPEDIATYYCQQHY |
| | | SIPFTFGQGTKLEIKRTVAAPSVFIFPPSDEQLKSGTASVVCLLNNFY |
| | | PREAKVQWKVDNALQSGNSQESVTEQDSKDSTYSLSSTLTLSKAD |
| | | YEKHKVYACEVTHQGLSSPVTKSFNRGECSPPSIPPHVQKSVNND |
| | | MIVTDNNGAVKFPQLCKFCDVRFSTCDNQKSCMSNCSITSICEKPQ |
| | | EVCVAVWRKNDENITLETVCHDPKLPYHDFILEDAASPKCIMKEK |
| | | KKPGETFFMCSCSSDECNDNIIFSEEYNTSNPD |
| 31 | L5 | DIQMTQSPSSLSASVGDRVTITCRASQDVSTAVDWYQQKPGKAPK |
| | | LLIYSASYRYTGVPDRFSGSGSGTDFTFTISSLQPEDIATYYCQQHY |
| | | SIPFTFGQGTKLEIKRTVAAPSVFIFPPSDEQLKSGTASVVCLLNNFY |
| | | PREAKVQWKVDNALQSGNSQESVTEQDSKDSTYSLSSTLTLSKAD |
| | | YEKHKVYACEVTHQGLSSPVTKSFNRGECSPPSPIPPHVQKSVNND |
| | | MIVTDNNGAVKFPQLCKFCDVRFSTCDNQKSCMSNCSITSICEKPQ |
| | | EVCVAVWRKNDENITLETVCHDPKLPYHDFILEDAASPKCIMKEK |
| | | KKPGETFFMCSCSSDECNDNIIFSEEYNTSNPD |
| 32 | L6 | DIQMTQSPSSLSASVGDRVTITCRASQDVSTAVDWYQQKPGKAPK |
| | | LLIYSASYRYTGVPDRFSGSGSGTDFTFTISSLQPEDIATYYCQQHY |
| | | SIPFTFGQGTKLEIKRTVAAPSVFIFPPSDEQLKSGTASVVCLLNNFY |
| | | PREAKVQWKVDNALQSGNSQESVTEQDSKDSTYSLSSTLTLSKAD |
| | | YEKHKVYACEVTHQGLSSPVTKSFNRGECGPPGPIPPHVQKSVNN |
| | | DMIVTDNNGAVKFPQLCKFCDVRFSTCDNQKSCMSNCSITSICEKP |
| | | QEVCVAVWRKNDENITLETVCHDPKLPYHDFILEDAASPKCIMKE |
| | | KKKPGETFFMCSCSSDECNDNIIFSEEYNTSNPD |
| 33 | L7 | DIQMTQSPSSLSASVGDRVTITCRASQDVSTAVDWYQQKPGKAPK |
| | | LLIYSASYRYTGVPDRFSGSGSGTDFTFTISSLQPEDIATYYCQQHY |
| | | SIPFTFGQGTKLEIKRTVAAPSVFIFPPSDEQLKSGTASVVCLLNNFY |
| | | PREAKVQWKVDNALQSGNSQESVTEQDSKDSTYSLSSTLTLSKAD |
| | | YEKHKVYACEVTHQGLSSPVTKSFNRGECDKTHTSPPIPPHVQKSV |
| | | NNDMIVTDNNGAVKFPQLCKFCDVRFSTCDNQKSCMSNCSITSICE |
| | | KPQEVCVAVWRKNDENITLETVCHDPKLPYHDFILEDAASPKCIM |
| | | KEKKKPGETFFMCSCSSDECNDNIIFSEEYNTSNPD |
| 34 | L8 | DIQMTQSPSSLSASVGDRVTITCRASQDVSTAVDWYQQKPGKAPK |
| | | LLIYSASYRYTGVPDRFSGSGSGTDFTFTISSLQPEDIATYYCQQHY |
| | | SIPFTFGQGTKLEIKRTVAAPSVFIFPPSDEQLKSGTASVVCLLNNFY |
| | | PREAKVQWKVDNALQSGNSQESVTEQDSKDSTYSLSSTLTLSKAD |
| | | YEKHKVYACEVTHQGLSSPVTKSFNRGECDKTHTSPPSPIPPHVQK |
| | | SVNNDMIVTDNNGAVKFPQLCKFCDVRFSTCDNQKSCMSNCSITSI |
| | | CEKPQEVCVAVWRKNDENITLETVCHDPKLPYHDFILEDAASPKCI |
| | | MKEKKKPGETFFMCSCSSDECNDNIIFSEEYNTSNPD |
| 35 | L9 | DIQMTQSPSSLSASVGDRVTITCRASQDVSTAVDWYQQKPGKAPK |
| | | LLIYSASYRYTGVPDRFSGSGSGTDFTFTISSLQPEDIATYYCQQHY |
| | | SIPFTFGQGTKLEIKRTVAAPSVFIFPPSDEQLKSGTASVVCLLNNFY |
| | | PREAKVQWKVDNALQSGNSQESVTEQDSKDSTYSLSSTLTLSKAD |
| | | YEKHKVYACEVTHQGLSSPVTKSFNRGECESKYGPPSPPSPIPPHV |
| | | QKSVNNDMIVTDNNGAVKFPQLCKFCDVRFSTCDNQKSCMSNCSI |
| | | TSICEKPQEVCVAVWRKNDENITLETVCHDPKLPYHDFILEDAASP |
| | | KCIMKEKKKPGETFFMCSCSSDECNDNIIFSEEYNTSNPD |
| 36 | L10 | DIQMTQSPSSLSASVGDRVTITCRASQDVSTAVDWYQQKPGKAPK |
| | | LLIYSASYRYTGVPDRFSGSGSGTDFTFTISSLQPEDIATYYCQQHY |
| | | SIPFTFGQGTKLEIKRTVAAPSVFIFPPSDEQLKSGTASVVCLLNNFY |
| | | PREAKVQWKVDNALQSGNSQESVTEQDSKDSTYSLSSTLTLSKAD |
| | | YEKHKVYACEVTHQGLSSPVTKSFNRGECGSIPPHVQKSVNNDMI |
| | | VTDNNGAVKFPQLCKFCDVRFSTCDNQKSCMSNCSITSICEKPQEV |
| | | CVAVWRKNDENITLETVCHDPKLPYHDFILEDAASPKCIMKEKKK |
| | | PGETFFMCSCSSDECNDNIIFSEEYNTSNPD |
| 37 | L11 | DIQMTQSPSSLSASVGDRVTITCRASQDVSTAVDWYQQKPGKAPK |
| | | LLIYSASYRYTGVPDRFSGSGSGTDFTFTISSLQPEDIATYYCQQHY |
| | | SIPFTFGQGTKLEIKRTVAAPSVFIFPPSDEQLKSGTASVVCLLNNFY |
| | | PREAKVQWKVDNALQSGNSQESVTEQDSKDSTYSLSSTLTLSKAD |
| | | YEKHKVYACEVTHQGLSSPVTKSFNRGECGGGSIPPHVQKSVNND |
| | | MIVTDNNGAVKFPQLCKFCDVRFSTCDNQKSCMSNCSITSICEKPQ |
| | | EVCVAVWRKNDENITLETVCHDPKLPYHDFILEDAASPKCIMKEK |
| | | KKPGETFFMCSCSSDECNDNIIFSEEYNTSNPD |
| 38 | H2 | gaagtccagctggtgcagagcggagccgaggtgaagaaaccaggcgcttctgtgaaggtgtcctgcaa |
| | | ggcctctgggtacatcttcaccggctacggcatcacctgggtgcggcaggctcctggccagggcctgga |
| | | atggatgggcgagatctttcccaggagagtgcagacctactactccgagaagttcaagggcagagtgac |
| | | catgaccaccgacacctccacctctaccgcctacatggaactgcggtctctgagatccgatgacaccgct |
| | | gtgtactactgcgccagagactacgacccttatttcgccctggattattggggccaaggcaccaccgtgac |
| | | agtctcctccgcctctaccaagggcccttccgtgttccccctggcccctagcagcaagtccacatcagga |
| | | ggcaccgctgctctgggctgcctggtcaaggactacttccctgaacctgtgaccgtgtcctggaactccg |
| | | gcgccctgacaagtggagtgcataccttccccgccgtgctgcagtcctctggcctgtactctctgtctagc |
| | | gtggtcactgtgccttcctctagcctcggcacacagacatacatctgcaacgtgaaccacaagccttccaa |
| | | caccaaagtggacaagagagtggaacccaagtcttgcgacaaaacccacacatgtccaccttgtcctgc |
| | | ccccgagctgctgggcggcccctccgtgtttctgtttcctcctaagccgaaggatacactgatgatctccc |
| | | ggacccctgaggtgacctgtgtggtggtggacgtgtctcacgaggaccccgaagtgaagttcaactggt |
| | | acgtggatggcgtggaagtgcacaatgctaagaccaagcctagagaagagcagtacaactccacctac |
| | | cgggtggtctctgtgctgaccgtcctgcatcaggactggctgaacggcaaagagtacaagtgcaaggtgt |
| | | ctaacaaggctctgcctgctcctatcgagaaaaccatctctaaggccaagggacagcctcgggaaccac |
| | | aagtgtacaccctgcctccttctagagaggagatgaccaagaaccaggtgagcctgacctgcctcgtgaa |
| | | aggcttctacccctctgacatcgccgtggagtgggagtccaatggccagcctgagaacaactacaagac |
| | | cacacctccagttctggactccgacggttccttcttcctgtactccaagctgaccgttgataagtccagatgg |
| | | cagcagggcaacgtgttctcctgttccgtgatgcacgaggccctgcacaaccactacacccagaagtcc |
| | | ctgagcttgtctcctggcgctggtggaggcggtagtggaggcggtggttcaggcggaggcggatctgg |
| | | cggaggtggctcaggcattcctcctcacgtgcagaaatccgtgaacaacgacatgatcgtgaccgacaa |
| | | caacggcgctgtgaagtttccacagctgtgcaagttctgcgacgtcagattctctacctgtgacaaccaga |
| | | agtcctgtatgtccaactgctccatcacctctatctgcgagaagccccaagaggtgtgcgtggccgtgtgg |
| | | cggaagaacgatgagaacatcaccctggaaaccgtgtgtcatgatcctaagctgccttaccacgacttcat |
| | | cctggaagatgccgcttctcccaagtgcatcatgaaagagaagaagaagcctggcgagacattcttcatg |
| | | tgctcttgtagctccgacgagtgcaacgacaatatcatcttctccgaggaatacaacaccagcaatcctga |
| | | c |
| 39 | H3 | gaagtccagctggtgcagagcggagccgaggtgaagaaaccaggcgcttctgtgaaggtgtcctgcaa |
| | | ggcctctgggtacatcttcaccggctacggcatcacctgggtgcggcaggctcctggccagggcctgga |
| | | atggatgggcgagatctttcccaggagagtgcagacctactactccgagaagttcaagggcagagtgac |
| | | catgaccaccgacacctccacctctaccgcctacatggaactgcggtctctgagatccgatgacaccgct |
| | | gtgtactactgcgccagagactacgacccttatttcgccctggattattggggccaaggcaccaccgtgac |
| | | agtctcctccgcctctaccaagggcccttccgtgttccccctggcccctagcagcaagtccacatcagga |
| | | ggcaccgctgctctgggctgcctggtcaaggactacttccctgaacctgtgaccgtgtcctggaactccg |
| | | gcgccctgacaagtggagtgcataccttccccgccgtgctgcagtcctctggcctgtactctctgtctagc |
| | | gtggtcactgtgccttcctctagcctcggcacacagacatacatctgcaacgtgaaccacaagccttccaa |
| | | caccaaagtggacaagagagtggaacccaagtcttgcgacaaaacccacacatgtccaccttgtcctgc |
| | | ccccgagctgctgggcggcccctccgtgtttctgtttcctcctaagccgaaggatacactgatgatctccc |
| | | ggacccctgaggtgacctgtgtggtggtggacgtgtctcacgaggaccccgaagtgaagttcaactggt |
| | | acgtggatggcgtggaagtgcacaatgctaagaccaagcctagagaagagcagtacaactccacctac |
| | | cgggtggtctctgtgctgaccgtcctgcatcaggactggctgaacggcaaagagtacaagtgcaaggtgt |
| | | ctaacaaggctctgcctgctcctatcgagaaaaccatctctaaggccaagggacagcctcgggaaccac |
| | | aagtgtacaccctgcctccttctagagaggagatgaccaagaaccaggtgagcctgacctgcctcgtgaa |
| | | aggcttctacccctctgacatcgccgtggagtgggagtccaatggccagcctgagaacaactacaagac |
| | | cacacctccagttctggactccgacggttccttcttcctgtactccaagctgaccgttgataagtccagatgg |
| | | cagcagggcaacgtgttctcctgttccgtgatgcacgaggccctgcacaaccactacacccagaagtcc |
| | | ctgagcttgtctcctggcgctggtggaggcggtagtggaggcggtggttcaggcggaggcggatctgg |
| | | cggaggtggctcaggccagtgcttctgccatctgtgcaccaaggacaacttcacctgcgtgaccgatggc |
| | | ctgtgttttgtgtccgtgaccgagacaaccgacaaagtgatccacaactccatgtgcatcgccgagatcga |
| | | cctgatccctagagatcggcccttcgtgtgtgctcctagctctaagaccggctctgtcaccacaacctactg |
| | | ctgtaatcaggaccactgcaacaagatcgagctgcctaccaccgtgaagtcctctccaggactgggccct |
| | | gtggaa |
| 40 | H4 | gaagtccagctggtgcagagcggagccgaggtgaagaaaccaggcgcttctgtgaaggtgtcctgcaa |
| | | ggcctctgggtacatcttcaccggctacggcatcacctgggtgcggcaggctcctggccagggcctgga |
| | | atggatgggcgagatctttcccaggagagtgcagacctactactccgagaagttcaagggcagagtgac |
| | | catgaccaccgacacctccacctctaccgcctacatggaactgcggtctctgagatccgatgacaccgct |
| | | gtgtactactgcgccagagactacgacccttatttcgccctggattattggggccaaggcaccaccgtgac |
| | | agtctcctccgcctctaccaagggcccttccgtgttccccctggcccctagcagcaagtccacatcagga |
| | | ggcaccgctgctctgggctgcctggtcaaggactacttccctgaacctgtgaccgtgtcctggaactccg |
| | | gcgccctgacaagtggagtgcataccttccccgccgtgctgcagtcctctggcctgtactctctgtctagc |
| | | gtggtcactgtgccttcctctagcctcggcacacagacatacatctgcaacgtgaaccacaagccttccaa |
| | | caccaaagtggacaagagagtggaacccaagtcttgcgacaaaacccacacatgtccaccttgtcctgc |
| | | ccccgagctgctgggcggcccctccgtgtttctgtttcctcctaagccgaaggatacactgatgatctccc |
| | | ggacccctgaggtgacctgtgtggtggtggacgtgtctcacgaggaccccgaagtgaagttcaactggt |
| | | acgtggatggcgtggaagtgcacaatgctaagaccaagcctagagaagagcagtacaactccacctac |
| | | cgggtggtctctgtgctgaccgtcctgcatcaggactggctgaacggcaaagagtacaagtgcaaggtgt |
| | | ctaacaaggctctgcctgctcctatcgagaaaaccatctctaaggccaagggacagcctcgggaaccac |
| | | aagtgtacaccctgcctccttctagagaggagatgaccaagaaccaggtgagcctgacctgcctcgtgaa |
| | | aggcttctacccctctgacatcgccgtggagtgggagtccaatggccagcctgagaacaactacaagac |
| | | cacacctccagttctggactccgacggttccttcttcctgtactccaagctgaccgttgataagtccagatgg |
| | | cagcagggcaacgtgttctcctgttccgtgatgcacgaggccctgcacaaccactacacccagaagtcc |
| | | ctgagcttgtctcctggcgctggtggaggcggtagtggaggcggtggttcaggcggaggcggatctgg |
| | | cggaggtggctcaggcattccacctcacgtgcagaagtccgtgaacaacgacatgatcgtgaccgacaa |
| | | caacggcgctgtcaagtttcctcagctgtgcaagttctgcgacgtgcggttctctacctgtgacaaccaga |
| | | aatcctgcatgtcccagtgttccatcacctctatctgcgagaagccccaagaggtgtgcgtggccgtgtgg |
| | | agaaagaacgacgagcagatcacactggaaaccgtgtgtcatgaccccaagctgccttaccacgacttc |
| | | atcctggaagatgccgcttctcctaagtgcatcatgaaagagaagaagaagcctggcgagaccttcttcat |
| | | gtgctcttgtagctccgacgagtgcaacgataacatcatcttctccgaggaataccagaccagcaatcctg |
| | | at |
| 41 | H5 | gaagtccagctggtgcagagcggagccgaggtgaagaaaccaggcgcttctgtgaaggtgtcctgcaa |
| | | ggcctctgggtacatcttcaccggctacggcatcacctgggtgcggcaggctcctggccagggcctgga |
| | | atggatgggcgagatctttcccaggagagtgcagacctactactccgagaagttcaagggcagagtgac |
| | | catgaccaccgacacctccacctctaccgcctacatggaactgcggtctctgagatccgatgacaccgct |
| | | gtgtactactgcgccagagactacgacccttatttcgccctggattattggggccaaggcaccaccgtgac |
| | | agtctcctccgcctctaccaagggcccttccgtgttccccctggcccctagcagcaagtccacatcagga |
| | | ggcaccgctgctctgggctgcctggtcaaggactacttccctgaacctgtgaccgtgtcctggaactccg |
| | | gcgccctgacaagtggagtgcataccttccccgccgtgctgcagtcctctggcctgtactctctgtctagc |
| | | gtggtcactgtgccttcctctagcctcggcacacagacatacatctgcaacgtgaaccacaagccttccaa |
| | | caccaaagtggacaagagagtggaacccaagtcttgcgacaaaacccacacatgtccaccttgtcctgc |
| | | ccccgagctgctgggcggcccctccgtgtttctgtttcctcctaagccgaaggatacactgatgatctccc |
| | | ggacccctgaggtgacctgtgtggtggtggacgtgtctcacgaggaccccgaagtgaagttcaactggt |
| | | acgtggatggcgtggaagtgcacaatgctaagaccaagcctagagaagagcagtacaactccacctac |
| | | cgggtggtctctgtgctgaccgtcctgcatcaggactggctgaacggcaaagagtacaagtgcaaggtgt |
| | | ctaacaaggctctgcctgctcctatcgagaaaaccatctctaaggccaagggacagcctcgggaaccac |
| | | aagtgtacaccctgcctccttctagagaggagatgaccaagaaccaggtgagcctgacctgcctcgtgaa |
| | | aggcttctacccctctgacatcgccgtggagtgggagtccaatggccagcctgagaacaactacaagac |
| | | cacacctccagttctggactccgacggttccttcttcctgtactccaagctgaccgttgataagtccagatgg |
| | | cagcagggcaacgtgttctcctgttccgtgatgcacgaggccctgcacaaccactacacccagaagtcc |
| | | ctgagcttgtctcctggcgctggtggaggcggtagtggaggcggtggttcaggcggaggcggatctgg |
| | | cggaggtggctcaggcattcctcctcacgtgcagaaatccgtgaacaacgacatgatcgtgaccgacaa |
| | | caacggcgctgtgaagtttccacagctgtgcaagttctgcgacgtcagattctctacctgtgacaaccaga |
| | | agtcctgtatgtccaactgctccatcacctctatctgcgagaagccccaagaggtgtgcgtggccgtgtgg |
| | | cggaagaacgatgagaacatcaccctggaaaccgtgtgtcatgatcctaagctgccttaccacgacttcat |
| | | cctggaagatgccgcttctcccaagtgcatcatgaaagagaagaagaagcctggcgagacattcttcatg |
| | | tgctcttgtagctccgacgagtgcaacgacaatatcatcttctccgaggaatacaacaccagcaatcctga |
| | | cggaggcggtggcagtggcggaggtggctcaggcggaggcggatctcagtgcttctgccatctgtgca |
| | | ccaaggacaacttcacctgcgtgaccgatggcctgtgttttgtgtccgtgaccgagacaaccgacaaagt |
| | | gatccacaactccatgtgcatcgccgagatcgacctgatccctagagatcggcccttcgtgtgtgctccta |
| | | gctctaagaccggctctgtcaccacaacctactgctgtaatcaggaccactgcaacaagatcgagctgcc |
| | | taccaccgtgaagtcctctccaggactgggccctgtggaa |
| 42 | H6 | gaagtccagctggtgcagagcggagccgaggtgaagaaaccaggcgcttctgtgaaggtgtcctgcaa |
| | | ggcctctgggtacatcttcaccggctacggcatcacctgggtgcggcaggctcctggccagggcctgga |
| | | atggatgggcgagatctttcccaggagagtgcagacctactactccgagaagttcaagggcagagtgac |
| | | catgaccaccgacacctccacctctaccgcctacatggaactgcggtctctgagatccgatgacaccgct |
| | | gtgtactactgcgccagagactacgacccttatttcgccctggattattggggccaaggcaccaccgtgac |
| | | agtctcctccgcctctaccaagggcccttccgtgttccccctggcccctagcagcaagtccacatcagga |
| | | ggcaccgctgctctgggctgcctggtcaaggactacttccctgaacctgtgaccgtgtcctggaactccg |
| | | gcgccctgacaagtggagtgcataccttccccgccgtgctgcagtcctctggcctgtactctctgtctagc |
| | | gtggtcactgtgccttcctctagcctcggcacacagacatacatctgcaacgtgaaccacaagccttccaa |
| | | caccaaagtggacaagagagtggaacccaagtcttgcgacaaaacccacacatgtccaccttgtcctgc |
| | | ccccgagctgctgggcggcccctccgtgtttctgtttcctcctaagccgaaggatacactgatgatctccc |
| | | ggacccctgaggtgacctgtgtggtggtggacgtgtctcacgaggaccccgaagtgaagttcaactggt |
| | | acgtggatggcgtggaagtgcacaatgctaagaccaagcctagagaagagcagtacaactccacctac |
| | | cgggtggtctctgtgctgaccgtcctgcatcaggactggctgaacggcaaagagtacaagtgcaaggtgt |
| | | ctaacaaggctctgcctgctcctatcgagaaaaccatctctaaggccaagggacagcctcgggaaccac |
| | | aagtgtacaccctgcctccttctagagaggagatgaccaagaaccaggtgagcctgacctgcctcgtgaa |
| | | aggcttctacccctctgacatcgccgtggagtgggagtccaatggccagcctgagaacaactacaagac |
| | | cacacctccagttctggactccgacggttccttcttcctgtactccaagctgaccgttgataagtccagatgg |
| | | cagcagggcaacgtgttctcctgttccgtgatgcacgaggccctgcacaaccactacacccagaagtcc |
| | | ctgagcttgtctcctggcaaggaacctaagtctagcgacaaaactcataccagcccccctagtccaattcc |
| | | tcctcacgtgcagaaatccgtgaacaacgacatgatcgtgaccgacaacaacggcgctgtgaagtttcca |
| | | cagctgtgcaagttctgcgacgtcagattctctacctgtgacaaccagaagtcctgtatgtccaactgctcc |
| | | atcacctctatctgcgagaagccccaagaggtgtgcgtggccgtgtggcggaagaacgatgagaacatc |
| | | accctggaaaccgtgtgtcatgatcctaagctgccttaccacgacttcatcctggaagatgccgcttctccc |
| | | aagtgcatcatgaaagagaagaagaagcctggcgagacattcttcatgtgctcttgtagctccgacgagt |
| | | gcaacgacaatatcatcttctccgaggaatacaacaccagcaatcctgac |
| 43 | L1 | gatatccagatgacccagtctcctagcagcctgagcgcttctgtgggcgacagagtgacaatcacctgta |
| | | gagcctctcaggacgtgtccaccgccgtggattggtaccagcagaagcccggcaaggctcctaagctg |
| | | ctgatctactctgcctcctaccggtacacaggagtccccgatagattctctggctccggctctggaaccga |
| | | cttcaccttcaccatctcctctctgcagcctgaggacattgccacctactactgccagcagcactactccatc |
| | | ccttttaccttcggccagggcaccaagctggaaatcaagcggaccgtggccgctccatccgtgttcatcttt |
| | | cctccttccgacgagcagctgaagtctggcaccgcttccgtggtgtgcctgctgaacaacttctaccctcg |
| | | ggaagccaaggtgcagtggaaagtggacaacgccctgcagtccggcaatagccaagagtccgtcacc |
| | | gagcaagactccaaggactctacctattctctctccagcacactgaccctgtctaaagccgactacgagaa |
| | | gcacaaggtgtacgcctgcgaagtgacccaccagggcctgtcttcccccgtgacaaagtccttcaacag |
| | | aggcgagtgtggtggaggcggtagtggaggcggtggttcaggcggaggcggatctggcggaggtgg |
| | | ctcaggcattcctcctcacgtgcagaaatccgtgaacaacgacatgatcgtgaccgacaacaacggcgct |
| | | gtgaagtttccacagctgtgcaagttctgcgacgtcagattctctacctgtgacaaccagaagtcctgtatgt |
| | | ccaactgctccatcacctctatctgcgagaagccccaagaggtgtgcgtggccgtgtggcggaagaacg |
| | | atgagaacatcaccctggaaaccgtgtgtcatgatcctaagctgccttaccacgacttcatcctggaagat |
| | | gccgcttctcccaagtgcatcatgaaagagaagaagaagcctggcgagacattcttcatgtgctcttgtag |
| | | ctccgacgagtgcaacgacaatatcatcttctccgaggaatacaacaccagcaatcctgac |
| 44 | L2 | gatatccagatgacccagtctcctagcagcctgagcgcttctgtgggcgacagagtgacaatcacctgta |
| | | gagcctctcaggacgtgtccaccgccgtggattggtaccagcagaagcccggcaaggctcctaagctg |
| | | ctgatctactctgcctcctaccggtacacaggagtccccgatagattctctggctccggctctggaaccga |
| | | cttcaccttcaccatctcctctctgcagcctgaggacattgccacctactactgccagcagcactactccatc |
| | | ccttttaccttcggccagggcaccaagctggaaatcaagcggaccgtggccgctccatccgtgttcatcttt |
| | | cctccttccgacgagcagctgaagtctggcaccgcttccgtggtgtgcctgctgaacaacttctaccctcg |
| | | ggaagccaaggtgcagtggaaagtggacaacgccctgcagtccggcaatagccaagagtccgtcacc |
| | | gagcaagactccaaggactctacctattctctctccagcacactgaccctgtctaaagccgactacgagaa |
| | | gcacaaggtgtacgcctgcgaagtgacccaccagggcctgtcttcccccgtgacaaagtccttcaacag |
| | | aggcgagtgtggtggaggcggtagtggaggcggtggttcaggcggaggcggatctggcggaggtgg |
| | | ctcaggccagtgcttctgccatctgtgcaccaaggacaacttcacctgcgtgaccgatggcctgtgttttgt |
| | | gtccgtgaccgagacaaccgacaaagtgatccacaactccatgtgcatcgccgagatcgacctgatccct |
| | | agagatcggcccttcgtgtgtgctcctagctctaagaccggctctgtcaccacaacctactgctgtaatcag |
| | | gaccactgcaacaagatcgagctgcctaccaccgtgaagtcctctccaggactgggccctgtggaa |
| 45 | L3 | gatatccagatgacccagtctcctagcagcctgagcgcttctgtgggcgacagagtgacaatcacctgta |
| | | gagcctctcaggacgtgtccaccgccgtggattggtaccagcagaagcccggcaaggctcctaagctg |
| | | ctgatctactctgcctcctaccggtacacaggagtccccgatagattctctggctccggctctggaaccga |
| | | cttcaccttcaccatctcctctctgcagcctgaggacattgccacctactactgccagcagcactactccatc |
| | | ccttttaccttcggccagggcaccaagctggaaatcaagcggaccgtggccgctccatccgtgttcatcttt |
| | | cctccttccgacgagcagctgaagtctggcaccgcttccgtggtgtgcctgctgaacaacttctaccctcg |
| | | ggaagccaaggtgcagtggaaagtggacaacgccctgcagtccggcaatagccaagagtccgtcacc |
| | | gagcaagactccaaggactctacctattctctctccagcacactgaccctgtctaaagccgactacgagaa |
| | | gcacaaggtgtacgcctgcgaagtgacccaccagggcctgtcttcccccgtgacaaagtccttcaacag |
| | | aggcgagtgtgaacctaagtctagcgacaaaactcataccagcccccctagtccaattcctcctcacgtgc |
| | | agaaatccgtgaacaacgacatgatcgtgaccgacaacaacggcgctgtgaagtttccacagctgtgca |
| | | agttctgcgacgtcagattctctacctgtgacaaccagaagtcctgtatgtccaactgctccatcacctctat |
| | | ctgcgagaagccccaagaggtgtgcgtggccgtgtggcggaagaacgatgagaacatcaccctggaa |
| | | accgtgtgtcatgatcctaagctgccttaccacgacttcatcctggaagatgccgcttctcccaagtgcatc |
| | | atgaaagagaagaagaagcctggcgagacattcttcatgtgctcttgtagctccgacgagtgcaacgaca |
| | | atatcatcttctccgaggaatacaacaccagcaatcctgac |
| 46 | L4 | gatatccagatgacccagtctcctagcagcctgagcgcttctgtgggcgacagagtgacaatcacctgta |
| | | gagcctctcaggacgtgtccaccgccgtggattggtaccagcagaagcccggcaaggctcctaagctg |
| | | ctgatctactctgcctcctaccggtacacaggagtccccgatagattctctggctccggctctggaaccga |
| | | cttcaccttcaccatctcctctctgcagcctgaggacattgccacctactactgccagcagcactactccatc |
| | | ccttttaccttcggccagggcaccaagctggaaatcaagcggaccgtggccgctccatccgtgttcatcttt |
| | | cctccttccgacgagcagctgaagtctggcaccgcttccgtggtgtgcctgctgaacaacttctaccctcg |
| | | ggaagccaaggtgcagtggaaagtggacaacgccctgcagtccggcaatagccaagagtccgtcacc |
| | | gagcaagactccaaggactctacctattctctctccagcacactgaccctgtctaaagccgactacgagaa |
| | | gcacaaggtgtacgcctgcgaagtgacccaccagggcctgtcttcccccgtgacaaagtccttcaacag |
| | | aggcgagtgtagcccccctagtattcctcctcacgtgcagaaatccgtgaacaacgacatgatcgtgacc |
| | | gacaacaacggcgctgtgaagtttccacagctgtgcaagttctgcgacgtcagattctctacctgtgacaa |
| | | ccagaagtcctgtatgtccaactgctccatcacctctatctgcgagaagccccaagaggtgtgcgtggcc |
| | | gtgtggcggaagaacgatgagaacatcaccctggaaaccgtgtgtcatgatcctaagctgccttaccacg |
| | | acttcatcctggaagatgccgcttctcccaagtgcatcatgaaagagaagaagaagcctggcgagacatt |
| | | cttcatgtgctcttgtagctccgacgagtgcaacgacaatatcatcttctccgaggaatacaacaccagcaa |
| | | tcctgac |
| 47 | L5 | gatatccagatgacccagtctcctagcagcctgagcgcttctgtgggcgacagagtgacaatcacctgta |
| | | gagcctctcaggacgtgtccaccgccgtggattggtaccagcagaagcccggcaaggctcctaagctg |
| | | ctgatctactctgcctcctaccggtacacaggagtccccgatagattctctggctccggctctggaaccga |
| | | cttcaccttcaccatctcctctctgcagcctgaggacattgccacctactactgccagcagcactactccatc |
| | | ccttttaccttcggccagggcaccaagctggaaatcaagcggaccgtggccgctccatccgtgttcatcttt |
| | | cctccttccgacgagcagctgaagtctggcaccgcttccgtggtgtgcctgctgaacaacttctaccctcg |
| | | ggaagccaaggtgcagtggaaagtggacaacgccctgcagtccggcaatagccaagagtccgtcacc |
| | | gagcaagactccaaggactctacctattctctctccagcacactgaccctgtctaaagccgactacgagaa |
| | | gcacaaggtgtacgcctgcgaagtgacccaccagggcctgtcttcccccgtgacaaagtccttcaacag |
| | | aggcgagtgtagcccccctagtccaattcctcctcacgtgcagaaatccgtgaacaacgacatgatcgtg |
| | | accgacaacaacggcgctgtgaagtttccacagctgtgcaagttctgcgacgtcagattctctacctgtga |
| | | caaccagaagtcctgtatgtccaactgctccatcacctctatctgcgagaagccccaagaggtgtgcgtg |
| | | gccgtgtggcggaagaacgatgagaacatcaccctggaaaccgtgtgtcatgatcctaagctgccttacc |
| | | acgacttcatcctggaagatgccgcttctcccaagtgcatcatgaaagagaagaagaagcctggcgaga |
| | | cattcttcatgtgctcttgtagctccgacgagtgcaacgacaatatcatcttctccgaggaatacaacacca |
| | | gcaatcctgac |
| 48 | L6 | gatatccagatgacccagtctcctagcagcctgagcgcttctgtgggcgacagagtgacaatcacctgta |
| | | gagcctctcaggacgtgtccaccgccgtggattggtaccagcagaagcccggcaaggctcctaagctg |
| | | ctgatctactctgcctcctaccggtacacaggagtccccgatagattctctggctccggctctggaaccga |
| | | cttcaccttcaccatctcctctctgcagcctgaggacattgccacctactactgccagcagcactactccatc |
| | | ccttttaccttcggccagggcaccaagctggaaatcaagcggaccgtggccgctccatccgtgttcatcttt |
| | | cctccttccgacgagcagctgaagtctggcaccgcttccgtggtgtgcctgctgaacaacttctaccctcg |
| | | ggaagccaaggtgcagtggaaagtggacaacgccctgcagtccggcaatagccaagagtccgtcacc |
| | | gagcaagactccaaggactctacctattctctctccagcacactgaccctgtctaaagccgactacgagaa |
| | | gcacaaggtgtacgcctgcgaagtgacccaccagggcctgtcttcccccgtgacaaagtccttcaacag |
| | | aggcgagtgtggaccacctggtccaattcctcctcacgtgcagaaatccgtgaacaacgacatgatcgtg |
| | | accgacaacaacggcgctgtgaagtttccacagctgtgcaagttctgcgacgtcagattctctacctgtga |
| | | caaccagaagtcctgtatgtccaactgctccatcacctctatctgcgagaagccccaagaggtgtgcgtg |
| | | gccgtgtggcggaagaacgatgagaacatcaccctggaaaccgtgtgtcatgatcctaagctgccttacc |
| | | acgacttcatcctggaagatgccgcttctcccaagtgcatcatgaaagagaagaagaagcctggcgaga |
| | | cattcttcatgtgctcttgtagctccgacgagtgcaacgacaatatcatcttctccgaggaatacaacacca |
| | | gcaatcctgac |
| 49 | L7 | gatatccagatgacccagtctcctagcagcctgagcgcttctgtgggcgacagagtgacaatcacctgta |
| | | gagcctctcaggacgtgtccaccgccgtggattggtaccagcagaagcccggcaaggctcctaagctg |
| | | ctgatctactctgcctcctaccggtacacaggagtccccgatagattctctggctccggctctggaaccga |
| | | cttcaccttcaccatctcctctctgcagcctgaggacattgccacctactactgccagcagcactactccatc |
| | | ccttttaccttcggccagggcaccaagctggaaatcaagcggaccgtggccgctccatccgtgttcatcttt |
| | | cctccttccgacgagcagctgaagtctggcaccgcttccgtggtgtgcctgctgaacaacttctaccctcg |
| | | ggaagccaaggtgcagtggaaagtggacaacgccctgcagtccggcaatagccaagagtccgtcacc |
| | | gagcaagactccaaggactctacctattctctctccagcacactgaccctgtctaaagccgactacgagaa |
| | | gcacaaggtgtacgcctgcgaagtgacccaccagggcctgtcttcccccgtgacaaagtccttcaacag |
| | | aggcgagtgtgacaaaactcataccagcccccctattcctcctcacgtgcagaaatccgtgaacaacgac |
| | | atgatcgtgaccgacaacaacggcgctgtgaagtttccacagctgtgcaagttctgcgacgtcagattctc |
| | | tacctgtgacaaccagaagtcctgtatgtccaactgctccatcacctctatctgcgagaagccccaagagg |
| | | tgtgcgtggccgtgtggcggaagaacgatgagaacatcaccctggaaaccgtgtgtcatgatcctaagct |
| | | gccttaccacgacttcatcctggaagatgccgcttctcccaagtgcatcatgaaagagaagaagaagcct |
| | | ggcgagacattcttcatgtgctcttgtagctccgacgagtgcaacgacaatatcatcttctccgaggaatac |
| | | aacaccagcaatcctgac |
| 50 | L8 | gatatccagatgacccagtctcctagcagcctgagcgcttctgtgggcgacagagtgacaatcacctgta |
| | | gagcctctcaggacgtgtccaccgccgtggattggtaccagcagaagcccggcaaggctcctaagctg |
| | | ctgatctactctgcctcctaccggtacacaggagtccccgatagattctctggctccggctctggaaccga |
| | | cttcaccttcaccatctcctctctgcagcctgaggacattgccacctactactgccagcagcactactccatc |
| | | ccttttaccttcggccagggcaccaagctggaaatcaagcggaccgtggccgctccatccgtgttcatcttt |
| | | cctccttccgacgagcagctgaagtctggcaccgcttccgtggtgtgcctgctgaacaacttctaccctcg |
| | | ggaagccaaggtgcagtggaaagtggacaacgccctgcagtccggcaatagccaagagtccgtcacc |
| | | gagcaagactccaaggactctacctattctctctccagcacactgaccctgtctaaagccgactacgagaa |
| | | gcacaaggtgtacgcctgcgaagtgacccaccagggcctgtcttcccccgtgacaaagtccttcaacag |
| | | aggcgagtgtgacaaaactcataccagcccccctagtccaattcctcctcacgtgcagaaatccgtgaac |
| | | aacgacatgatcgtgaccgacaacaacggcgctgtgaagtttccacagctgtgcaagttctgcgacgtca |
| | | gattctctacctgtgacaaccagaagtcctgtatgtccaactgctccatcacctctatctgcgagaagcccc |
| | | aagaggtgtgcgtggccgtgtggcggaagaacgatgagaacatcaccctggaaaccgtgtgtcatgatc |
| | | ctaagctgccttaccacgacttcatcctggaagatgccgcttctcccaagtgcatcatgaaagagaagaag |
| | | aagcctggcgagacattcttcatgtgctcttgtagctccgacgagtgcaacgacaatatcatcttctccgag |
| | | gaatacaacaccagcaatcctgac |
| 51 | L9 | gatatccagatgacccagtctcctagcagcctgagcgcttctgtgggcgacagagtgacaatcacctgta |
| | | gagcctctcaggacgtgtccaccgccgtggattggtaccagcagaagcccggcaaggctcctaagctg |
| | | ctgatctactctgcctcctaccggtacacaggagtccccgatagattctctggctccggctctggaaccga |
| | | cttcaccttcaccatctcctctctgcagcctgaggacattgccacctactactgccagcagcactactccatc |
| | | ccttttaccttcggccagggcaccaagctggaaatcaagcggaccgtggccgctccatccgtgttcatcttt |
| | | cctccttccgacgagcagctgaagtctggcaccgcttccgtggtgtgcctgctgaacaacttctaccctcg |
| | | ggaagccaaggtgcagtggaaagtggacaacgccctgcagtccggcaatagccaagagtccgtcacc |
| | | gagcaagactccaaggactctacctattctctctccagcacactgaccctgtctaaagccgactacgagaa |
| | | gcacaaggtgtacgcctgcgaagtgacccaccagggcctgtcttcccccgtgacaaagtccttcaacag |
| | | aggcgagtgtgagagcaagtacggaccaccttctccaccatctccaattcctcctcacgtgcagaaatcc |
| | | gtgaacaacgacatgatcgtgaccgacaacaacggcgctgtgaagtttccacagctgtgcaagttctgcg |
| | | acgtcagattctctacctgtgacaaccagaagtcctgtatgtccaactgctccatcacctctatctgcgagaa |
| | | gccccaagaggtgtgcgtggccgtgtggcggaagaacgatgagaacatcaccctggaaaccgtgtgtc |
| | | atgatcctaagctgccttaccacgacttcatcctggaagatgccgcttctcccaagtgcatcatgaaagaga |
| | | agaagaagcctggcgagacattcttcatgtgctcttgtagctccgacgagtgcaacgacaatatcatcttct |
| | | ccgaggaatacaacaccagcaatcctgac |
| 52 | L10 | gatatccagatgacccagtctcctagcagcctgagcgcttctgtgggcgacagagtgacaatcacctgta |
| | | gagcctctcaggacgtgtccaccgccgtggattggtaccagcagaagcccggcaaggctcctaagctg |
| | | ctgatctactctgcctcctaccggtacacaggagtccccgatagattctctggctccggctctggaaccga |
| | | cttcaccttcaccatctcctctctgcagcctgaggacattgccacctactactgccagcagcactactccatc |
| | | ccttttaccttcggccagggcaccaagctggaaatcaagcggaccgtggccgctccatccgtgttcatcttt |
| | | cctccttccgacgagcagctgaagtctggcaccgcttccgtggtgtgcctgctgaacaacttctaccctcg |
| | | ggaagccaaggtgcagtggaaagtggacaacgccctgcagtccggcaatagccaagagtccgtcacc |
| | | gagcaagactccaaggactctacctattctctctccagcacactgaccctgtctaaagccgactacgagaa |
| | | gcacaaggtgtacgcctgcgaagtgacccaccagggcctgtcttcccccgtgacaaagtccttcaacag |
| | | aggcgagtgtggctcaattcctcctcacgtgcagaaatccgtgaacaacgacatgatcgtgaccgacaac |
| | | aacggcgctgtgaagtttccacagctgtgcaagttctgcgacgtcagattctctacctgtgacaaccagaa |
| | | gtcctgtatgtccaactgctccatcacctctatctgcgagaagccccaagaggtgtgcgtggccgtgtggc |
| | | ggaagaacgatgagaacatcaccctggaaaccgtgtgtcatgatcctaagctgccttaccacgacttcatc |
| | | ctggaagatgccgcttctcccaagtgcatcatgaaagagaagaagaagcctggcgagacattcttcatgt |
| | | gctcttgtagctccgacgagtgcaacgacaatatcatcttctccgaggaatacaacaccagcaatcctgac |
| 53 | L11 | gatatccagatgacccagtctcctagcagcctgagcgcttctgtgggcgacagagtgacaatcacctgta |
| | | gagcctctcaggacgtgtccaccgccgtggattggtaccagcagaagcccggcaaggctcctaagctg |
| | | ctgatctactctgcctcctaccggtacacaggagtccccgatagattctctggctccggctctggaaccga |
| | | cttcaccttcaccatctcctctctgcagcctgaggacattgccacctactactgccagcagcactactccatc |
| | | ccttttaccttcggccagggcaccaagctggaaatcaagcggaccgtggccgctccatccgtgttcatcttt |
| | | cctccttccgacgagcagctgaagtctggcaccgcttccgtggtgtgcctgctgaacaacttctaccctcg |
| | | ggaagccaaggtgcagtggaaagtggacaacgccctgcagtccggcaatagccaagagtccgtcacc |
| | | gagcaagactccaaggactctacctattctctctccagcacactgaccctgtctaaagccgactacgagaa |
| | | gcacaaggtgtacgcctgcgaagtgacccaccagggcctgtcttcccccgtgacaaagtccttcaacag |
| | | aggcgagtgtggtggaggcagtattcctcctcacgtgcagaaatccgtgaacaacgacatgatcgtgacc |
| | | gacaacaacggcgctgtgaagtttccacagctgtgcaagttctgcgacgtcagattctctacctgtgacaa |
| | | ccagaagtcctgtatgtccaactgctccatcacctctatctgcgagaagccccaagaggtgtgcgtggcc |
| | | gtgtggcggaagaacgatgagaacatcaccctggaaaccgtgtgtcatgatcctaagctgccttaccacg |
| | | acttcatcctggaagatgccgcttctcccaagtgcatcatgaaagagaagaagaagcctggcgagacatt |
| | | cttcatgtgctcttgtagctccgacgagtgcaacgacaatatcatcttctccgaggaatacaacaccagcaa |
| | | tcctgac |

Exemplary secretory signal peptide

| | | |
|---|---|---|
| 54 | Secretory | MGWSCIILFLVATATGVhS |
| | signal | |
| | peptide | |
| 55 | Secretory | atgggctggtcctgcatcatcctgttcctggtggctaccgccaccggcgtgcactcc |
| | signal | |
| | peptide | |

Linkers

| | | |
|---|---|---|
| 56 | G9-linker | GGSGGGGSG |
| 57 | E-linker | EPKSSDKTHTSPPSP |
| 58 | E2-linker | ERKSSVESPPSP |
| 59 | G21-linker | GGGGSGGGGSGGGGSGGGGSG |
| 60 | G15-linker | GGGSGGGGSGGGGSG |
| 61 | G4-linker | GGGS |
| 62 | G2-linker | GS |
| 63 | E4-linker | SPPS |
| 64 | E5-linker | SPPSP |
| 65 | E5a-linker | GPPGP |
| 66 | E8-linker | DKTHTSPP |
| 67 | E10-linker | DKTHTSPPSP |
| 68 | E12-linker | ESKYGPPSPPSP |
| 69 | G9-linker | ggcggatctggcggaggtggctcaggc |
| 70 | E-linker | gaacctaagtctagcgacaaaactcataccagcccccctagtcca |
| 71 | E2-linker | gaaaggaagtctagcgtggaatctccacctagtcca |
| 72 | G21-linker | ggtggaggcggtagtggaggcggtggttcaggcggaggcggatctggcggaggtggctcaggc |
| 73 | G15-linker | ggcggtggttcaggcggaggcggatctggcggaggtggctcaggc |
| 74 | G4-linker | ggtggaggcagt |
| 75 | G2-linker | ggctca |
| 76 | E4-linker | agcccccctagt |
| 77 | E5-linker | Agcccccctagtcca |
| 78 | E5 a-linker | ggaccacctggtcca |
| 79 | E8-linker | gacaaaactcataccagcccccct |
| 80 | E10-linker | gacaaaactcataccagcccccctagtcca |
| 81 | E12-linker | gagagcaagtacggaccaccttctccaccatctcca |
| 82 | Exemplary | (GGGGS)ₙ, n is between 1 and 8. |
| | linker | |
| 83 | Exemplary | (GGGGS)₆ |
| | linker | |
| 84 | Exemplary | (GSTSGSGKPGSGEGS)ₙ, n is between 1 and 3. |
| | linker | |
| 85 | Exemplary | (G)ₙ |
| | linker | |
| 86 | Exemplary | (GS) |
| | linker | |
| 87 | Exemplary | (GSGGS)ₙ |
| | linker | |
| 88 | Exemplary | (GGGS)ₙ |
| | linker | |

Additional Exemplary TGFβ receptor ectodomain sequences (e.g., truncated forms)

| | | |
|---|---|---|
| 89 | TGFβR1 | ALQCFCHLCTKDNFTCVTDGLCFVSVTETTDKVIHNSMCIAEIDLIP |
| | | RDRPFVCAPSSKTGSVTTTYCCNQDHCNKIEL |
| 90 | TGFβR1 | AALLPGATALQCFCHLCTKDNFTCVTDGLCFVSVTETTDKVIHNS |
| | | MCIAEIDLIPRDRPFVCAPSSKTGSVTTTYCCNQDHCNKIELPTTVK |
| | | SSPGLGPVE |
| 91 | TGFβR2 | QLCKFCDVRFSTCDNQKSCMSNCSITSICEKPQEVCVAVWRKNDE |
| | | NITLETVCHDPKLPYHDFILEDAASPKCIMKEKKKPGETFFMCSCSS |
| | | DECNDNIIF |
| 92 | TGFβR2 | IPPHVQKSVNNDMIVTDNNGAVKFPQLCKFCDVRFSTCDNQKSCM |
| | | SNCSITSICEKPQEVCVAVWRKNDENITLETVCHDPKLPYHDFILED |
| | | AASPKCIMKEKKKPGETFFMCSCSSDECNDNIIF |
| 93 | TGFβR2 | QLCKFCDVRFSTCDNQKSCMSNCSITSICEKPQEVCVAVWRKNDE |
| | | NITLETVCHDPKLPYHDFILEDAASPKCIMKEKKKPGETFFMCSCSS |
| | | DECNDNIIFSEEYNTSNPD |

1. A fusion protein comprising:
a) a full-length antibody that specifically binds to PD-L1 and comprises two heavy chains and two light chains, wherein each of the two heavy chains comprises a heavy chain variable region (V_{H}) and each of the two light chains comprises a light chain variable region (V_{L});
b) a TGFβ superfamily receptor ectodomain;
wherein the TGFβ superfamily receptor ectodomain is fused to at least one of the heavy chains or light chains of the anti-PD-L1 antibody.

2. The fusion protein of claim 1, wherein the TGFβ superfamily receptor ectodomain is fused to the C-terminus or the N-terminus of the at least one or both of the two light chains.

3.-4. (canceled)

5. The fusion protein of claim 1, wherein the TGFβ superfamily receptor ectodomain is fused to the C-terminus or the N-terminus of the at least one or both of the two heavy chains.

6.-7. (canceled)

8. The fusion protein of claim 1, wherein the TGFβ superfamily receptor ectodomain comprises a TGFβ receptor type II ectodomain or a variant thereof.

9. The fusion protein of claim 8, wherein the TGFβ receptor type II ectodomain or a variant thereof comprises a human TGFβ receptor type II ectodomain or a variant thereof, wherein the human TGFβ receptor type II ectodomain or a variant thereof comprises the amino acid sequence of SEQ ID NO: 2 or a variant that has at least about 90% sequence identity to SEQ ID NO: 2.

10. (canceled)

11. The fusion protein of claim 9, wherein the human TGFβ receptor type II ectodomain or a variant thereof comprises the amino acid sequence of SEQ ID NO: 2 or SEQ ID NO: 3.

12. (canceled)

13. The fusion protein of claim 1, wherein the TGFβ superfamily receptor ectodomain comprises a TGFβ receptor type I ectodomain or a variant thereof.

14. (canceled)

15. The fusion protein of claim 1, wherein:
a) the V_{H} comprises:
i) a HC-CDR1 comprising an amino acid sequence of SEQ ID NO: 6;
ii) a HC-CDR2 comprising an amino acid sequence of SEQ ID NO: 7; and
iii) a HC-CDR3 comprising an amino acid sequence of SEQ ID NO: 8; and,
b) the V_{L} comprises:
i) a LC-CDR1 comprising an amino acid sequence of SEQ ID NO: 9;
ii) a LC-CDR2 comprising an amino acid sequence of SEQ ID NO: 10; and
iii) a LC-CDR3 comprising an amino acid sequence of SEQ ID NO: 11.

16. The fusion protein of claim 1, wherein the TGFβ superfamily receptor ectodomain is fused to at least one of the heavy chains or light chains of the anti-PD-L1 antibody via a linker.

17. The fusion protein of claim 16, wherein the linker is a peptide linker.

18. (canceled)

19. The fusion protein of claim 16, wherein the linker is a GS linker or comprises a modified sequence derived from the hinge region of an IgG.

20. (canceled)

21. The fusion protein of claim 1, wherein the V_{H} comprises the amino acid sequence of SEQ ID NO: 12 or a variant thereof having at least about 80% sequence identity, and wherein the V_{L} comprises the amino acid sequence of SEQ ID NO: 13 or a variant thereof having at least about 80% sequence identity.

22. The fusion protein of claim 1, wherein the heavy chain comprises the amino acid sequence of any one of SEQ ID NOs: 14-17 or a variant thereof having at least about 80% sequence identity, and wherein the light chain comprises the amino acid sequence of SEQ ID NO: 18 or a variant thereof having at least about 80% sequence identity.

23. (canceled)

24. The fusion protein of claim 2, wherein the TGFβ superfamily receptor ectodomain or the polypeptide fused to at least one or both of the two light chains comprises the amino acid sequence of any one of SEQ ID NOs: 27-37 or a variant thereof having at least about 80% sequence identity; or the TGFβ superfamily receptor ectodomain fused to at least one or both of the two heavy chains comprises the amino acid sequence of any one of SEQ ID NOs: 22-26 or a variant thereof having at least about 80% sequence identity.

25. (canceled)

26. The fusion protein of claim 24, wherein:
a) the TGFβ superfamily receptor ectodomain is fused to both of the two heavy chains comprising the amino acid sequence of SEQ ID NO: 22, and the two light chains comprise the amino acid sequence of SEQ ID NO: 18;
b) the TGFβ superfamily receptor ectodomain is fused to both of the two heavy chains comprising the amino acid sequence of SEQ ID NO: 23, and the two light chains comprise the amino acid sequence of SEQ ID NO: 18;
c) the TGFβ superfamily receptor ectodomain is fused to both of the two light chains comprising the amino acid sequence of SEQ ID NO: 27, and both of the two heavy chains comprises the amino acid sequence of SEQ ID NO: 16 or 17;
d) the TGFβ superfamily receptor ectodomain is fused to both of the two light chains comprising the amino acid sequence of SEQ ID NO: 28, and both of the two heavy chains comprises the amino acid sequence of SEQ ID NO: 16 or 17;
e) the TGFβ superfamily receptor ectodomain is fused to both of the two heavy chains comprising the amino acid sequence of SEQ ID NO: 22, and the TGFβ superfamily receptor ectodomain is also fused to both of the two light chains comprising the amino acid sequence of SEQ ID NO: 28;
f) the TGFβ superfamily receptor ectodomain is fused to both of the two heavy chains comprising the amino acid sequence of SEQ ID NO: 23, and the TGFβ superfamily receptor ectodomain is also fused to both of the two light chains comprising the amino acid sequence of SEQ ID NO: 27;
g) the TGFβ superfamily receptor ectodomain is fused to both of the two heavy chains comprising the amino acid sequence of SEQ ID NO: 22, and the TGFβ superfamily receptor ectodomain is also fused to both of the two light chains comprising the amino acid sequence of SEQ ID NO: 27;
h) the TGFβ superfamily receptor ectodomain is fused to both of the two heavy chains comprising the amino acid sequence of SEQ ID NO: 23, and the TGFβ superfamily receptor ectodomain is also fused to both of the two light chains comprising the amino acid sequence of SEQ ID NO: 28;
i) the polypeptide is fused to both of the two heavy chains comprising the amino acid sequence of SEQ ID NO: 24, and both of the two light chains comprise the amino acid sequence of SEQ ID NO: 18;
j) the TGFβ superfamily receptor ectodomain is fused to both of the two heavy chains comprising the amino acid sequence of SEQ ID NO: 25, and both of the two light chains comprise the amino acid sequence of SEQ ID NO: 18;
k) the TGFβ superfamily receptor ectodomain is fused to both of the two heavy chains comprising the amino acid sequence of SEQ ID NO: 26, and both of the two light chains comprise the amino acid sequence of SEQ ID NO: 18;
l) the TGFβ superfamily receptor ectodomain is fused to both of the two light chains comprising the amino acid sequence of SEQ ID NO: 29, and both of the two heavy chains comprise the amino acid sequence of SEQ ID NO: 16 or 17;
m) the TGFβ superfamily receptor ectodomain is fused to both of the two light chains comprising the amino acid sequence of SEQ ID NO: 30, and both of the two heavy chains comprise the amino acid sequence of SEQ ID NO: 16 or 17;
n) the TGFβ superfamily receptor ectodomain is fused to both of the two light chains comprising the amino acid sequence of SEQ ID NO: 31, and both of the two heavy chains comprise the amino acid sequence of SEQ ID NO: 16 or 17;
o) the TGFβ superfamily receptor ectodomain is fused to both of the two light chains comprising the amino acid sequence of SEQ ID NO: 32, and both of the two heavy chains comprise the amino acid sequence of SEQ ID NO: 16 or 17;
p) the TGFβ superfamily receptor ectodomain is fused to both of the two light chains comprising the amino acid sequence of SEQ ID NO: 33, and both of the two heavy chains comprise the amino acid sequence of SEQ ID NO: 16 or 17;
q) the TGFβ superfamily receptor ectodomain is fused to both of the two light chains comprising the amino acid sequence of SEQ ID NO: 34, and both of the two heavy chains comprise the amino acid sequence of SEQ ID NO: 16 or 17;
r) the TGFβ superfamily receptor ectodomain is fused to both of the two light chains comprising the amino acid sequence of SEQ ID NO: 35 and both of the two heavy chains comprise the amino acid sequence of SEQ ID NO: 16 or 17;
s) the TGFβ superfamily receptor ectodomain is fused to both of the two light chains comprising the amino acid sequence of SEQ ID NO: 36 and both of the two heavy chains comprise the amino acid sequence of SEQ ID NO: 16 or 17; or
t) the TGFβ superfamily receptor ectodomain is fused to both of the two light chains comprising the amino acid sequence of SEQ ID NO: 37 and both of the two heavy chains comprise the amino acid sequence of SEQ ID NO: 16 or 17.

27.-33. (canceled)

34. A pharmaceutical composition comprising the fusion protein of claim 1.

35. A nucleic acid encoding the fusion protein or a portion thereof of claim 1.

36.-39. (canceled)

40. A method of treating a disease or condition in an individual, comprising administering to the individual an effective amount of the fusion protein of claim 1.

41. The method of claim 40, wherein the disease or condition is a cancer.

42. The method of claim 41, wherein the cancer is non-small cell lung cancer (NSCLC), small cell lung cancer (SCLC), cervical cancer, oropharyngeal cancer, anal cancer, vaginal or penile cancer, biliary tract cancer, cholangiocarcinoma, gallbladder cancer, head and neck cancer, pancreas cancer, prostate cancer, urothelial cancer, bladder cancer, genitourinary cancer, urogenital cancer, gastric cancer, breast cancer or colorectal cancer.

43.-47. (canceled)


SEQUENCE LISTING (93 sequences)

SEQ ID NO: 1 - protein, 91 aa, Artificial Sequence
  Synthetic Construct
QCFCHLCTKDNFTCVTDGLCFVSVTETTDKVIHNSMCIAEIDLIPRDRPFVCAPSSKTGSVTTTYCCNQDHCNKIELPTTVKSSPGLGPVE

SEQ ID NO: 2 - protein, 136 aa, Artificial Sequence
  Synthetic Construct
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 3 - protein, 136 aa, Artificial Sequence
  Synthetic Construct
IPPHVQKSVNNDMIVTDNNGAVKFPQLCKFCDVRFSTCDNQKSCMSQCSITSICEKPQEVCVAVWRKNDEQITLETVCHDPKLPYHDFILEDAASPKCIMKEKKKPGETFFMCSCSSDECNDNIIFSEEYQTSNPD

SEQ ID NO: 4 - DNA, 273 bp, Artificial Sequence
  Synthetic Construct
cagtgcttctgccatctgtgcaccaaggacaacttcacctgcgtgaccgatggcctgtgttttgtgtccgtgaccgagacaaccgacaaagtgatccacaactccatgtgcatcgccgagatcgacctgatccctagagatcggcccttcgtgtgtgctcctagctctaagaccggctctgtcaccacaacctactgctgtaatcaggaccactgcaacaagatcgagctgcctaccaccgtgaagtcctctccaggactgggccctgtggaa

SEQ ID NO: 5 - DNA, 408 bp, Artificial Sequence
  Synthetic Construct
attcctcctcacgtgcagaaatccgtgaacaacgacatgatcgtgaccgacaacaacggcgctgtgaagtttccacagctgtgcaagttctgcgacgtcagattctctacctgtgacaaccagaagtcctgtatgtccaactgctccatcacctctatctgcgagaagccccaagaggtgtgcgtggccgtgtggcggaagaacgatgagaacatcaccctggaaaccgtgtgtcatgatcctaagctgccttaccacgacttcatcctggaagatgccgcttctcccaagtgcatcatgaaagagaagaagaagcctggcgagacattcttcatgtgctcttgtagctccgacgagtgcaacgacaatatcatcttctccgaggaatacaacaccagcaatcctgac

SEQ ID NO: 6 - protein, 5 aa, Artificial Sequence
  Synthetic Construct
GYGIT

SEQ ID NO: 7 - protein, 17 aa, Artificial Sequence
  Synthetic Construct
EIFPRRVQTYYSEKFKG

SEQ ID NO: 8 - protein, 10 aa, Artificial Sequence
  Synthetic Construct
DYDPYFALDY

SEQ ID NO: 9 - protein, 11 aa, Artificial Sequence
  Synthetic Construct
RASQDVSTAVD

SEQ ID NO: 10 - protein, 7 aa, Artificial Sequence
  Synthetic Construct
SASYRYT

SEQ ID NO: 11 - protein, 9 aa, Artificial Sequence
  Synthetic Construct
QQHYSIPFT

SEQ ID NO: 12 - protein, 119 aa, Artificial Sequence
  Synthetic Construct
EVQLVQSGAEVKKPGASVKVSCKASGYIFTGYGITWVRQAPGQGLEWMGEIFPRRVQTYYSEKFKGRVTMTTDTSTSTAYMELRSLRSDDTAVYYCARDYDPYFALDYWGQGTTVTVSS

SEQ ID NO: 13 - protein, 110 aa, Artificial Sequence
  Synthetic Construct
DIQMTQSPSSLSASVGDRVTITCRASQDVSTAVDWYQQKPGKAPKLLIYSASYRYTGVPDRFSGSGSGTDFTFTISSLQPEDIATYYCQQHYSIPFTFGQGTKLEIKRTV

SEQ ID NO: 14 - protein, 449 aa, Artificial Sequence
  Synthetic Construct
EVQLVQSGAEVKKPGASVKVSCKASGYIFTGYGITWVRQAPGQGLEWMGEIFPRRVQTYYSEKFKGRVTMTTDTSTSTAYMELRSLRSDDTAVYYCARDYDPYFALDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 449 aa, Artificial Sequence
  Synthetic Construct
EVQLVQSGAEVKKPGASVKVSCKASGYIFTGYGITWVRQAPGQGLEWMGEIFPRRVQTYYSEKFKGRVTMTTDTSTSTAYMELRSLRSDDTAVYYCARDYDPYFALDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGA

SEQ ID NO: 16 - protein, 449 aa, Artificial Sequence
  Synthetic Construct
EVQLVQSGAEVKKPGASVKVSCKASGYIFTGYGITWVRQAPGQGLEWMGEIFPRRVQTYYSEKFKGRVTMTTDTSTSTAYMELRSLRSDDTAVYYCARDYDPYFALDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 449 aa, Artificial Sequence
  Synthetic Construct
EVQLVQSGAEVKKPGASVKVSCKASGYIFTGYGITWVRQAPGQGLEWMGEIFPRRVQTYYSEKFKGRVTMTTDTSTSTAYMELRSLRSDDTAVYYCARDYDPYFALDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGA

SEQ ID NO: 18 - protein, 214 aa, Artificial Sequence
  Synthetic Construct
DIQMTQSPSSLSASVGDRVTITCRASQDVSTAVDWYQQKPGKAPKLLIYSASYRYTGVPDRFSGSGSGTDFTFTISSLQPEDIATYYCQQHYSIPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - DNA, 1347 bp, Artificial Sequence
  Synthetic Construct
gaagtccagctggtgcagagcggagccgaggtgaagaaaccaggcgcttctgtgaaggtgtcctgcaaggcctctgggtacatcttcaccggctacggcatcacctgggtgcggcaggctcctggccagggcctggaatggatgggcgagatctttcccaggagagtgcagacctactactccgagaagttcaagggcagagtgaccatgaccaccgacacctccacctctaccgcctacatggaactgcggtctctgagatccgatgacaccgctgtgtactactgcgccagagactacgacccttatttcgccctggattattggggccaaggcaccaccgtgacagtctcctccgcctctaccaagggcccttccgtgttccccctggcccctagcagcaagtccacatcaggaggcaccgctgctctgggctgcctggtcaaggactacttccctgaacctgtgaccgtgtcctggaactccggcgccctgacaagtggagtgcataccttccccgccgtgctgcagtcctctggcctgtactctctgtctagcgtggtcactgtgccttcctctagcctcggcacacagacatacatctgcaacgtgaaccacaagccttccaacaccaaagtggacaagaaggtggaacccaagtcttgcgacaaaacccacacatgtccaccttgtcctgcccccgagctgctgggcggcccctccgtgtttctgtttcctcctaagccgaaggatacactgatgatctcccggacccctgaggtgacctgtgtggtggtggacgtgtctcacgaggaccccgaagtgaagttcaactggtacgtggatggcgtggaagtgcacaatgctaagaccaagcctagagaagagcagtacgcctccacctaccgggtggtctctgtgctgaccgtcctgcatcaggactggctgaacggcaaagagtacaagtgcaaggtgtctaacaaggctctgcctgctcctatcgagaaaaccatctctaaggccaagggacagcctcgggaaccacaagtgtacaccctgcctccttctagagaggagatgaccaagaaccaggtgagcctgacctgcctcgtgaaaggcttctacccctctgacatcgccgtggagtgggagtccaatggccagcctgagaacaactacaagaccacacctccagttctggactccgacggttccttcttcctgtactccaagctgaccgttgataagtccagatggcagcagggcaacgtgttctcctgttccgtgatgcacgaggccctgcacaaccactacacccagaagtccctgagcttgtctcctggcaag

SEQ ID NO: 20 - DNA, 1347 bp, Artificial Sequence
  Synthetic Construct
gaagtccagctggtgcagagcggagccgaggtgaagaaaccaggcgcttctgtgaaggtgtcctgcaaggcctctgggtacatcttcaccggctacggcatcacctgggtgcggcaggctcctggccagggcctggaatggatgggcgagatctttcccaggagagtgcagacctactactccgagaagttcaagggcagagtgaccatgaccaccgacacctccacctctaccgcctacatggaactgcggtctctgagatccgatgacaccgctgtgtactactgcgccagagactacgacccttatttcgccctggattattggggccaaggcaccaccgtgacagtctcctccgcctctaccaagggcccttccgtgttccccctggcccctagcagcaagtccacatcaggaggcaccgctgctctgggctgcctggtcaaggactacttccctgaacctgtgaccgtgtcctggaactccggcgccctgacaagtggagtgcataccttccccgccgtgctgcagtcctctggcctgtactctctgtctagcgtggtcactgtgccttcctctagcctcggcacacagacatacatctgcaacgtgaaccacaagccttccaacaccaaagtggacaagagagtggaacccaagtcttgcgacaaaacccacacatgtccaccttgtcctgcccccgagctgctgggcggcccctccgtgtttctgtttcctcctaagccgaaggatacactgatgatctcccggacccctgaggtgacctgtgtggtggtggacgtgtctcacgaggaccccgaagtgaagttcaactggtacgtggatggcgtggaagtgcacaatgctaagaccaagcctagagaagagcagtacaactccacctaccgggtggtctctgtgctgaccgtcctgcatcaggactggctgaacggcaaagagtacaagtgcaaggtgtctaacaaggctctgcctgctcctatcgagaaaaccatctctaaggccaagggacagcctcgggaaccacaagtgtacaccctgcctccttctagagaggagatgaccaagaaccaggtgagcctgacctgcctcgtgaaaggcttctacccctctgacatcgccgtggagtgggagtccaatggccagcctgagaacaactacaagaccacacctccagttctggactccgacggttccttcttcctgtactccaagctgaccgttgataagtccagatggcagcagggcaacgtgttctcctgttccgtgatgcacgaggccctgcacaaccactacacccagaagtccctgagcttgtctcctggcaag

SEQ ID NO: 21 - DNA, 642 bp, Artificial Sequence
  Synthetic Construct
gatatccagatgacccagtctcctagcagcctgagcgcttctgtgggcgacagagtgacaatcacctgtagagcctctcaggacgtgtccaccgccgtggattggtaccagcagaagcccggcaaggctcctaagctgctgatctactctgcctcctaccggtacacaggagtccccgatagattctctggctccggctctggaaccgacttcaccttcaccatctcctctctgcagcctgaggacattgccacctactactgccagcagcactactccatcccttttaccttcggccagggcaccaagctggaaatcaagcggaccgtggccgctccatccgtgttcatctttcctccttccgacgagcagctgaagtctggcaccgcttccgtggtgtgcctgctgaacaacttctaccctcgggaagccaaggtgcagtggaaagtggacaacgccctgcagtccggcaatagccaagagtccgtcaccgagcaagactccaaggactctacctattctctctccagcacactgaccctgtctaaagccgactacgagaagcacaaggtgtacgcctgcgaagtgacccaccagggcctgtcttcccccgtgacaaagtccttcaacagaggcgagtgt

SEQ ID NO: 22 - protein, 606 aa, Artificial Sequence
  Synthetic Construct
EVQLVQSGAEVKKPGASVKVSCKASGYIFTGYGITWVRQAPGQGLEWMGEIFPRRVQTYYSEKFKGRVTMTTDTSTSTAYMELRSLRSDDTAVYYCARDYDPYFALDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSGGGGSGIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 23 - protein, 561 aa, Artificial Sequence
  Synthetic Construct
EVQLVQSGAEVKKPGASVKVSCKASGYIFTGYGITWVRQAPGQGLEWMGEIFPRRVQTYYSEKFKGRVTMTTDTSTSTAYMELRSLRSDDTAVYYCARDYDPYFALDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSGGGGSGQCFCHLCTKDNFTCVTDGLCFVSVTETTDKVIHNSMCIAEIDLIPRDRPFVCAPSSKTGSVTTTYCCNQDHCNKIELPTTVKSSPGLGPVE

SEQ ID NO: 24 - protein, 606 aa, Artificial Sequence
  Synthetic Construct
EVQLVQSGAEVKKPGASVKVSCKASGYIFTGYGITWVRQAPGQGLEWMGEIFPRRVQTYYSEKFKGRVTMTTDTSTSTAYMELRSLRSDDTAVYYCARDYDPYFALDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSGGGGSGIPPHVQKSVNNDMIVTDNNGAVKFPQLCKFCDVRFSTCDNQKSCMSQCSITSICEKPQEVCVAVWRKNDEQITLETVCHDPKLPYHDFILEDAASPKCIMKEKKKPGETFFMCSCSSDECNDNIIFSEEYQTSNPD

SEQ ID NO: 25 - protein, 712 aa, Artificial Sequence
  Synthetic Construct
EVQLVQSGAEVKKPGASVKVSCKASGYIFTGYGITWVRQAPGQGLEWMGEIFPRRVQTYYSEKFKGRVTMTTDTSTSTAYMELRSLRSDDTAVYYCARDYDPYFALDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSGGGGSGIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDGGGGSGGGGSGGGGSQCFCHLCTKDNFTCVTDGLCFVSVTETTDKVIHNSMCIAEIDLIPRDRPFVCAPSSKTGSVTTTYCCNQDHCNKIELPTTVKSSPGLGPVE

SEQ ID NO: 26 - protein, 600 aa, Artificial Sequence
  Synthetic Construct
EVQLVQSGAEVKKPGASVKVSCKASGYIFTGYGITWVRQAPGQGLEWMGEIFPRRVQTYYSEKFKGRVTMTTDTSTSTAYMELRSLRSDDTAVYYCARDYDPYFALDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKEPKSSDKTHTSPPSPIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 27 - protein, 371 aa, Artificial Sequence
  Synthetic Construct
DIQMTQSPSSLSASVGDRVTITCRASQDVSTAVDWYQQKPGKAPKLLIYSASYRYTGVPDRFSGSGSGTDFTFTISSLQPEDIATYYCQQHYSIPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 28 - protein, 326 aa, Artificial Sequence
  Synthetic Construct
DIQMTQSPSSLSASVGDRVTITCRASQDVSTAVDWYQQKPGKAPKLLIYSASYRYTGVPDRFSGSGSGTDFTFTISSLQPEDIATYYCQQHYSIPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGQCFCHLCTKDNFTCVTDGLCFVSVTETTDKVIHNSMCIAEIDLIPRDRPFVCAPSSKTGSVTTTYCCNQDHCNKIELPTTVKSSPGLGPVE

SEQ ID NO: 29 - protein, 365 aa, Artificial Sequence
  Synthetic Construct
DIQMTQSPSSLSASVGDRVTITCRASQDVSTAVDWYQQKPGKAPKLLIYSASYRYTGVPDRFSGSGSGTDFTFTISSLQPEDIATYYCQQHYSIPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECEPKSSDKTHTSPPSPIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 30 - protein, 354 aa, Artificial Sequence
  Synthetic Construct
DIQMTQSPSSLSASVGDRVTITCRASQDVSTAVDWYQQKPGKAPKLLIYSASYRYTGVPDRFSGSGSGTDFTFTISSLQPEDIATYYCQQHYSIPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECSPPSIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 31 - protein, 355 aa, Artificial Sequence
  Synthetic Construct
DIQMTQSPSSLSASVGDRVTITCRASQDVSTAVDWYQQKPGKAPKLLIYSASYRYTGVPDRFSGSGSGTDFTFTISSLQPEDIATYYCQQHYSIPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECSPPSPIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 32 - protein, 355 aa, Artificial Sequence
  Synthetic Construct
DIQMTQSPSSLSASVGDRVTITCRASQDVSTAVDWYQQKPGKAPKLLIYSASYRYTGVPDRFSGSGSGTDFTFTISSLQPEDIATYYCQQHYSIPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGPPGPIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 33 - protein, 358 aa, Artificial Sequence
  Synthetic Construct
DIQMTQSPSSLSASVGDRVTITCRASQDVSTAVDWYQQKPGKAPKLLIYSASYRYTGVPDRFSGSGSGTDFTFTISSLQPEDIATYYCQQHYSIPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTSPPIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 34 - protein, 360 aa, Artificial Sequence
  Synthetic Construct
DIQMTQSPSSLSASVGDRVTITCRASQDVSTAVDWYQQKPGKAPKLLIYSASYRYTGVPDRFSGSGSGTDFTFTISSLQPEDIATYYCQQHYSIPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTSPPSPIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 35 - protein, 362 aa, Artificial Sequence
  Synthetic Construct
DIQMTQSPSSLSASVGDRVTITCRASQDVSTAVDWYQQKPGKAPKLLIYSASYRYTGVPDRFSGSGSGTDFTFTISSLQPEDIATYYCQQHYSIPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECESKYGPPSPPSPIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 36 - protein, 352 aa, Artificial Sequence
  Synthetic Construct
DIQMTQSPSSLSASVGDRVTITCRASQDVSTAVDWYQQKPGKAPKLLIYSASYRYTGVPDRFSGSGSGTDFTFTISSLQPEDIATYYCQQHYSIPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGSIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 37 - protein, 354 aa, Artificial Sequence
  Synthetic Construct
DIQMTQSPSSLSASVGDRVTITCRASQDVSTAVDWYQQKPGKAPKLLIYSASYRYTGVPDRFSGSGSGTDFTFTISSLQPEDIATYYCQQHYSIPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGSIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 38 - DNA, 1818 bp, Artificial Sequence
  Synthetic Construct
gaagtccagctggtgcagagcggagccgaggtgaagaaaccaggcgcttctgtgaaggtgtcctgcaaggcctctgggtacatcttcaccggctacggcatcacctgggtgcggcaggctcctggccagggcctggaatggatgggcgagatctttcccaggagagtgcagacctactactccgagaagttcaagggcagagtgaccatgaccaccgacacctccacctctaccgcctacatggaactgcggtctctgagatccgatgacaccgctgtgtactactgcgccagagactacgacccttatttcgccctggattattggggccaaggcaccaccgtgacagtctcctccgcctctaccaagggcccttccgtgttccccctggcccctagcagcaagtccacatcaggaggcaccgctgctctgggctgcctggtcaaggactacttccctgaacctgtgaccgtgtcctggaactccggcgccctgacaagtggagtgcataccttccccgccgtgctgcagtcctctggcctgtactctctgtctagcgtggtcactgtgccttcctctagcctcggcacacagacatacatctgcaacgtgaaccacaagccttccaacaccaaagtggacaagagagtggaacccaagtcttgcgacaaaacccacacatgtccaccttgtcctgcccccgagctgctgggcggcccctccgtgtttctgtttcctcctaagccgaaggatacactgatgatctcccggacccctgaggtgacctgtgtggtggtggacgtgtctcacgaggaccccgaagtgaagttcaactggtacgtggatggcgtggaagtgcacaatgctaagaccaagcctagagaagagcagtacaactccacctaccgggtggtctctgtgctgaccgtcctgcatcaggactggctgaacggcaaagagtacaagtgcaaggtgtctaacaaggctctgcctgctcctatcgagaaaaccatctctaaggccaagggacagcctcgggaaccacaagtgtacaccctgcctccttctagagaggagatgaccaagaaccaggtgagcctgacctgcctcgtgaaaggcttctacccctctgacatcgccgtggagtgggagtccaatggccagcctgagaacaactacaagaccacacctccagttctggactccgacggttccttcttcctgtactccaagctgaccgttgataagtccagatggcagcagggcaacgtgttctcctgttccgtgatgcacgaggccctgcacaaccactacacccagaagtccctgagcttgtctcctggcgctggtggaggcggtagtggaggcggtggttcaggcggaggcggatctggcggaggtggctcaggcattcctcctcacgtgcagaaatccgtgaacaacgacatgatcgtgaccgacaacaacggcgctgtgaagtttccacagctgtgcaagttctgcgacgtcagattctctacctgtgacaaccagaagtcctgtatgtccaactgctccatcacctctatctgcgagaagccccaagaggtgtgcgtggccgtgtggcggaagaacgatgagaacatcaccctggaaaccgtgtgtcatgatcctaagctgccttaccacgacttcatcctggaagatgccgcttctcccaagtgcatcatgaaagagaagaagaagcctggcgagacattcttcatgtgctcttgtagctccgacgagtgcaacgacaatatcatcttctccgaggaatacaacaccagcaatcctgac

SEQ ID NO: 39 - DNA, 1683 bp, Artificial Sequence
  Synthetic Construct
gaagtccagctggtgcagagcggagccgaggtgaagaaaccaggcgcttctgtgaaggtgtcctgcaaggcctctgggtacatcttcaccggctacggcatcacctgggtgcggcaggctcctggccagggcctggaatggatgggcgagatctttcccaggagagtgcagacctactactccgagaagttcaagggcagagtgaccatgaccaccgacacctccacctctaccgcctacatggaactgcggtctctgagatccgatgacaccgctgtgtactactgcgccagagactacgacccttatttcgccctggattattggggccaaggcaccaccgtgacagtctcctccgcctctaccaagggcccttccgtgttccccctggcccctagcagcaagtccacatcaggaggcaccgctgctctgggctgcctggtcaaggactacttccctgaacctgtgaccgtgtcctggaactccggcgccctgacaagtggagtgcataccttccccgccgtgctgcagtcctctggcctgtactctctgtctagcgtggtcactgtgccttcctctagcctcggcacacagacatacatctgcaacgtgaaccacaagccttccaacaccaaagtggacaagagagtggaacccaagtcttgcgacaaaacccacacatgtccaccttgtcctgcccccgagctgctgggcggcccctccgtgtttctgtttcctcctaagccgaaggatacactgatgatctcccggacccctgaggtgacctgtgtggtggtggacgtgtctcacgaggaccccgaagtgaagttcaactggtacgtggatggcgtggaagtgcacaatgctaagaccaagcctagagaagagcagtacaactccacctaccgggtggtctctgtgctgaccgtcctgcatcaggactggctgaacggcaaagagtacaagtgcaaggtgtctaacaaggctctgcctgctcctatcgagaaaaccatctctaaggccaagggacagcctcgggaaccacaagtgtacaccctgcctccttctagagaggagatgaccaagaaccaggtgagcctgacctgcctcgtgaaaggcttctacccctctgacatcgccgtggagtgggagtccaatggccagcctgagaacaactacaagaccacacctccagttctggactccgacggttccttcttcctgtactccaagctgaccgttgataagtccagatggcagcagggcaacgtgttctcctgttccgtgatgcacgaggccctgcacaaccactacacccagaagtccctgagcttgtctcctggcgctggtggaggcggtagtggaggcggtggttcaggcggaggcggatctggcggaggtggctcaggccagtgcttctgccatctgtgcaccaaggacaacttcacctgcgtgaccgatggcctgtgttttgtgtccgtgaccgagacaaccgacaaagtgatccacaactccatgtgcatcgccgagatcgacctgatccctagagatcggcccttcgtgtgtgctcctagctctaagaccggctctgtcaccacaacctactgctgtaatcaggaccactgcaacaagatcgagctgcctaccaccgtgaagtcctctccaggactgggccctgtggaa

SEQ ID NO: 40 - DNA, 1818 bp, Artificial Sequence
  Synthetic Construct
gaagtccagctggtgcagagcggagccgaggtgaagaaaccaggcgcttctgtgaaggtgtcctgcaaggcctctgggtacatcttcaccggctacggcatcacctgggtgcggcaggctcctggccagggcctggaatggatgggcgagatctttcccaggagagtgcagacctactactccgagaagttcaagggcagagtgaccatgaccaccgacacctccacctctaccgcctacatggaactgcggtctctgagatccgatgacaccgctgtgtactactgcgccagagactacgacccttatttcgccctggattattggggccaaggcaccaccgtgacagtctcctccgcctctaccaagggcccttccgtgttccccctggcccctagcagcaagtccacatcaggaggcaccgctgctctgggctgcctggtcaaggactacttccctgaacctgtgaccgtgtcctggaactccggcgccctgacaagtggagtgcataccttccccgccgtgctgcagtcctctggcctgtactctctgtctagcgtggtcactgtgccttcctctagcctcggcacacagacatacatctgcaacgtgaaccacaagccttccaacaccaaagtggacaagagagtggaacccaagtcttgcgacaaaacccacacatgtccaccttgtcctgcccccgagctgctgggcggcccctccgtgtttctgtttcctcctaagccgaaggatacactgatgatctcccggacccctgaggtgacctgtgtggtggtggacgtgtctcacgaggaccccgaagtgaagttcaactggtacgtggatggcgtggaagtgcacaatgctaagaccaagcctagagaagagcagtacaactccacctaccgggtggtctctgtgctgaccgtcctgcatcaggactggctgaacggcaaagagtacaagtgcaaggtgtctaacaaggctctgcctgctcctatcgagaaaaccatctctaaggccaagggacagcctcgggaaccacaagtgtacaccctgcctccttctagagaggagatgaccaagaaccaggtgagcctgacctgcctcgtgaaaggcttctacccctctgacatcgccgtggagtgggagtccaatggccagcctgagaacaactacaagaccacacctccagttctggactccgacggttccttcttcctgtactccaagctgaccgttgataagtccagatggcagcagggcaacgtgttctcctgttccgtgatgcacgaggccctgcacaaccactacacccagaagtccctgagcttgtctcctggcgctggtggaggcggtagtggaggcggtggttcaggcggaggcggatctggcggaggtggctcaggcattccacctcacgtgcagaagtccgtgaacaacgacatgatcgtgaccgacaacaacggcgctgtcaagtttcctcagctgtgcaagttctgcgacgtgcggttctctacctgtgacaaccagaaatcctgcatgtcccagtgttccatcacctctatctgcgagaagccccaagaggtgtgcgtggccgtgtggagaaagaacgacgagcagatcacactggaaaccgtgtgtcatgaccccaagctgccttaccacgacttcatcctggaagatgccgcttctcctaagtgcatcatgaaagagaagaagaagcctggcgagaccttcttcatgtgctcttgtagctccgacgagtgcaacgataacatcatcttctccgaggaataccagaccagcaatcctgat

SEQ ID NO: 41 - DNA, 2136 bp, Artificial Sequence
  Synthetic Construct
gaagtccagctggtgcagagcggagccgaggtgaagaaaccaggcgcttctgtgaaggtgtcctgcaaggcctctgggtacatcttcaccggctacggcatcacctgggtgcggcaggctcctggccagggcctggaatggatgggcgagatctttcccaggagagtgcagacctactactccgagaagttcaagggcagagtgaccatgaccaccgacacctccacctctaccgcctacatggaactgcggtctctgagatccgatgacaccgctgtgtactactgcgccagagactacgacccttatttcgccctggattattggggccaaggcaccaccgtgacagtctcctccgcctctaccaagggcccttccgtgttccccctggcccctagcagcaagtccacatcaggaggcaccgctgctctgggctgcctggtcaaggactacttccctgaacctgtgaccgtgtcctggaactccggcgccctgacaagtggagtgcataccttccccgccgtgctgcagtcctctggcctgtactctctgtctagcgtggtcactgtgccttcctctagcctcggcacacagacatacatctgcaacgtgaaccacaagccttccaacaccaaagtggacaagagagtggaacccaagtcttgcgacaaaacccacacatgtccaccttgtcctgcccccgagctgctgggcggcccctccgtgtttctgtttcctcctaagccgaaggatacactgatgatctcccggacccctgaggtgacctgtgtggtggtggacgtgtctcacgaggaccccgaagtgaagttcaactggtacgtggatggcgtggaagtgcacaatgctaagaccaagcctagagaagagcagtacaactccacctaccgggtggtctctgtgctgaccgtcctgcatcaggactggctgaacggcaaagagtacaagtgcaaggtgtctaacaaggctctgcctgctcctatcgagaaaaccatctctaaggccaagggacagcctcgggaaccacaagtgtacaccctgcctccttctagagaggagatgaccaagaaccaggtgagcctgacctgcctcgtgaaaggcttctacccctctgacatcgccgtggagtgggagtccaatggccagcctgagaacaactacaagaccacacctccagttctggactccgacggttccttcttcctgtactccaagctgaccgttgataagtccagatggcagcagggcaacgtgttctcctgttccgtgatgcacgaggccctgcacaaccactacacccagaagtccctgagcttgtctcctggcgctggtggaggcggtagtggaggcggtggttcaggcggaggcggatctggcggaggtggctcaggcattcctcctcacgtgcagaaatccgtgaacaacgacatgatcgtgaccgacaacaacggcgctgtgaagtttccacagctgtgcaagttctgcgacgtcagattctctacctgtgacaaccagaagtcctgtatgtccaactgctccatcacctctatctgcgagaagccccaagaggtgtgcgtggccgtgtggcggaagaacgatgagaacatcaccctggaaaccgtgtgtcatgatcctaagctgccttaccacgacttcatcctggaagatgccgcttctcccaagtgcatcatgaaagagaagaagaagcctggcgagacattcttcatgtgctcttgtagctccgacgagtgcaacgacaatatcatcttctccgaggaatacaacaccagcaatcctgacggaggcggtggcagtggcggaggtggctcaggcggaggcggatctcagtgcttctgccatctgtgcaccaaggacaacttcacctgcgtgaccgatggcctgtgttttgtgtccgtgaccgagacaaccgacaaagtgatccacaactccatgtgcatcgccgagatcgacctgatccctagagatcggcccttcgtgtgtgctcctagctctaagaccggctctgtcaccacaacctactgctgtaatcaggaccactgcaacaagatcgagctgcctaccaccgtgaagtcctctccaggactgggccctgtggaa

SEQ ID NO: 42 - DNA, 1800 bp, Artificial Sequence
  Synthetic Construct
gaagtccagctggtgcagagcggagccgaggtgaagaaaccaggcgcttctgtgaaggtgtcctgcaaggcctctgggtacatcttcaccggctacggcatcacctgggtgcggcaggctcctggccagggcctggaatggatgggcgagatctttcccaggagagtgcagacctactactccgagaagttcaagggcagagtgaccatgaccaccgacacctccacctctaccgcctacatggaactgcggtctctgagatccgatgacaccgctgtgtactactgcgccagagactacgacccttatttcgccctggattattggggccaaggcaccaccgtgacagtctcctccgcctctaccaagggcccttccgtgttccccctggcccctagcagcaagtccacatcaggaggcaccgctgctctgggctgcctggtcaaggactacttccctgaacctgtgaccgtgtcctggaactccggcgccctgacaagtggagtgcataccttccccgccgtgctgcagtcctctggcctgtactctctgtctagcgtggtcactgtgccttcctctagcctcggcacacagacatacatctgcaacgtgaaccacaagccttccaacaccaaagtggacaagagagtggaacccaagtcttgcgacaaaacccacacatgtccaccttgtcctgcccccgagctgctgggcggcccctccgtgtttctgtttcctcctaagccgaaggatacactgatgatctcccggacccctgaggtgacctgtgtggtggtggacgtgtctcacgaggaccccgaagtgaagttcaactggtacgtggatggcgtggaagtgcacaatgctaagaccaagcctagagaagagcagtacaactccacctaccgggtggtctctgtgctgaccgtcctgcatcaggactggctgaacggcaaagagtacaagtgcaaggtgtctaacaaggctctgcctgctcctatcgagaaaaccatctctaaggccaagggacagcctcgggaaccacaagtgtacaccctgcctccttctagagaggagatgaccaagaaccaggtgagcctgacctgcctcgtgaaaggcttctacccctctgacatcgccgtggagtgggagtccaatggccagcctgagaacaactacaagaccacacctccagttctggactccgacggttccttcttcctgtactccaagctgaccgttgataagtccagatggcagcagggcaacgtgttctcctgttccgtgatgcacgaggccctgcacaaccactacacccagaagtccctgagcttgtctcctggcaaggaacctaagtctagcgacaaaactcataccagcccccctagtccaattcctcctcacgtgcagaaatccgtgaacaacgacatgatcgtgaccgacaacaacggcgctgtgaagtttccacagctgtgcaagttctgcgacgtcagattctctacctgtgacaaccagaagtcctgtatgtccaactgctccatcacctctatctgcgagaagccccaagaggtgtgcgtggccgtgtggcggaagaacgatgagaacatcaccctggaaaccgtgtgtcatgatcctaagctgccttaccacgacttcatcctggaagatgccgcttctcccaagtgcatcatgaaagagaagaagaagcctggcgagacattcttcatgtgctcttgtagctccgacgagtgcaacgacaatatcatcttctccgaggaatacaacaccagcaatcctgac

SEQ ID NO: 43 - DNA, 1113 bp, Artificial Sequence
  Synthetic Construct
gatatccagatgacccagtctcctagcagcctgagcgcttctgtgggcgacagagtgacaatcacctgtagagcctctcaggacgtgtccaccgccgtggattggtaccagcagaagcccggcaaggctcctaagctgctgatctactctgcctcctaccggtacacaggagtccccgatagattctctggctccggctctggaaccgacttcaccttcaccatctcctctctgcagcctgaggacattgccacctactactgccagcagcactactccatcccttttaccttcggccagggcaccaagctggaaatcaagcggaccgtggccgctccatccgtgttcatctttcctccttccgacgagcagctgaagtctggcaccgcttccgtggtgtgcctgctgaacaacttctaccctcgggaagccaaggtgcagtggaaagtggacaacgccctgcagtccggcaatagccaagagtccgtcaccgagcaagactccaaggactctacctattctctctccagcacactgaccctgtctaaagccgactacgagaagcacaaggtgtacgcctgcgaagtgacccaccagggcctgtcttcccccgtgacaaagtccttcaacagaggcgagtgtggtggaggcggtagtggaggcggtggttcaggcggaggcggatctggcggaggtggctcaggcattcctcctcacgtgcagaaatccgtgaacaacgacatgatcgtgaccgacaacaacggcgctgtgaagtttccacagctgtgcaagttctgcgacgtcagattctctacctgtgacaaccagaagtcctgtatgtccaactgctccatcacctctatctgcgagaagccccaagaggtgtgcgtggccgtgtggcggaagaacgatgagaacatcaccctggaaaccgtgtgtcatgatcctaagctgccttaccacgacttcatcctggaagatgccgcttctcccaagtgcatcatgaaagagaagaagaagcctggcgagacattcttcatgtgctcttgtagctccgacgagtgcaacgacaatatcatcttctccgaggaatacaacaccagcaatcctgac

SEQ ID NO: 44 - DNA, 978 bp, Artificial Sequence
  Synthetic Construct
gatatccagatgacccagtctcctagcagcctgagcgcttctgtgggcgacagagtgacaatcacctgtagagcctctcaggacgtgtccaccgccgtggattggtaccagcagaagcccggcaaggctcctaagctgctgatctactctgcctcctaccggtacacaggagtccccgatagattctctggctccggctctggaaccgacttcaccttcaccatctcctctctgcagcctgaggacattgccacctactactgccagcagcactactccatcccttttaccttcggccagggcaccaagctggaaatcaagcggaccgtggccgctccatccgtgttcatctttcctccttccgacgagcagctgaagtctggcaccgcttccgtggtgtgcctgctgaacaacttctaccctcgggaagccaaggtgcagtggaaagtggacaacgccctgcagtccggcaatagccaagagtccgtcaccgagcaagactccaaggactctacctattctctctccagcacactgaccctgtctaaagccgactacgagaagcacaaggtgtacgcctgcgaagtgacccaccagggcctgtcttcccccgtgacaaagtccttcaacagaggcgagtgtggtggaggcggtagtggaggcggtggttcaggcggaggcggatctggcggaggtggctcaggccagtgcttctgccatctgtgcaccaaggacaacttcacctgcgtgaccgatggcctgtgttttgtgtccgtgaccgagacaaccgacaaagtgatccacaactccatgtgcatcgccgagatcgacctgatccctagagatcggcccttcgtgtgtgctcctagctctaagaccggctctgtcaccacaacctactgctgtaatcaggaccactgcaacaagatcgagctgcctaccaccgtgaagtcctctccaggactgggccctgtggaa

SEQ ID NO: 45 - DNA, 1095 bp, Artificial Sequence
  Synthetic Construct
gatatccagatgacccagtctcctagcagcctgagcgcttctgtgggcgacagagtgacaatcacctgtagagcctctcaggacgtgtccaccgccgtggattggtaccagcagaagcccggcaaggctcctaagctgctgatctactctgcctcctaccggtacacaggagtccccgatagattctctggctccggctctggaaccgacttcaccttcaccatctcctctctgcagcctgaggacattgccacctactactgccagcagcactactccatcccttttaccttcggccagggcaccaagctggaaatcaagcggaccgtggccgctccatccgtgttcatctttcctccttccgacgagcagctgaagtctggcaccgcttccgtggtgtgcctgctgaacaacttctaccctcgggaagccaaggtgcagtggaaagtggacaacgccctgcagtccggcaatagccaagagtccgtcaccgagcaagactccaaggactctacctattctctctccagcacactgaccctgtctaaagccgactacgagaagcacaaggtgtacgcctgcgaagtgacccaccagggcctgtcttcccccgtgacaaagtccttcaacagaggcgagtgtgaacctaagtctagcgacaaaactcataccagcccccctagtccaattcctcctcacgtgcagaaatccgtgaacaacgacatgatcgtgaccgacaacaacggcgctgtgaagtttccacagctgtgcaagttctgcgacgtcagattctctacctgtgacaaccagaagtcctgtatgtccaactgctccatcacctctatctgcgagaagccccaagaggtgtgcgtggccgtgtggcggaagaacgatgagaacatcaccctggaaaccgtgtgtcatgatcctaagctgccttaccacgacttcatcctggaagatgccgcttctcccaagtgcatcatgaaagagaagaagaagcctggcgagacattcttcatgtgctcttgtagctccgacgagtgcaacgacaatatcatcttctccgaggaatacaacaccagcaatcctgac

SEQ ID NO: 46 - DNA, 1062 bp, Artificial Sequence
  Synthetic Construct
gatatccagatgacccagtctcctagcagcctgagcgcttctgtgggcgacagagtgacaatcacctgtagagcctctcaggacgtgtccaccgccgtggattggtaccagcagaagcccggcaaggctcctaagctgctgatctactctgcctcctaccggtacacaggagtccccgatagattctctggctccggctctggaaccgacttcaccttcaccatctcctctctgcagcctgaggacattgccacctactactgccagcagcactactccatcccttttaccttcggccagggcaccaagctggaaatcaagcggaccgtggccgctccatccgtgttcatctttcctccttccgacgagcagctgaagtctggcaccgcttccgtggtgtgcctgctgaacaacttctaccctcgggaagccaaggtgcagtggaaagtggacaacgccctgcagtccggcaatagccaagagtccgtcaccgagcaagactccaaggactctacctattctctctccagcacactgaccctgtctaaagccgactacgagaagcacaaggtgtacgcctgcgaagtgacccaccagggcctgtcttcccccgtgacaaagtccttcaacagaggcgagtgtagcccccctagtattcctcctcacgtgcagaaatccgtgaacaacgacatgatcgtgaccgacaacaacggcgctgtgaagtttccacagctgtgcaagttctgcgacgtcagattctctacctgtgacaaccagaagtcctgtatgtccaactgctccatcacctctatctgcgagaagccccaagaggtgtgcgtggccgtgtggcggaagaacgatgagaacatcaccctggaaaccgtgtgtcatgatcctaagctgccttaccacgacttcatcctggaagatgccgcttctcccaagtgcatcatgaaagagaagaagaagcctggcgagacattcttcatgtgctcttgtagctccgacgagtgcaacgacaatatcatcttctccgaggaatacaacaccagcaatcctgac

SEQ ID NO: 47 - DNA, 1065 bp, Artificial Sequence
  Synthetic Construct
gatatccagatgacccagtctcctagcagcctgagcgcttctgtgggcgacagagtgacaatcacctgtagagcctctcaggacgtgtccaccgccgtggattggtaccagcagaagcccggcaaggctcctaagctgctgatctactctgcctcctaccggtacacaggagtccccgatagattctctggctccggctctggaaccgacttcaccttcaccatctcctctctgcagcctgaggacattgccacctactactgccagcagcactactccatcccttttaccttcggccagggcaccaagctggaaatcaagcggaccgtggccgctccatccgtgttcatctttcctccttccgacgagcagctgaagtctggcaccgcttccgtggtgtgcctgctgaacaacttctaccctcgggaagccaaggtgcagtggaaagtggacaacgccctgcagtccggcaatagccaagagtccgtcaccgagcaagactccaaggactctacctattctctctccagcacactgaccctgtctaaagccgactacgagaagcacaaggtgtacgcctgcgaagtgacccaccagggcctgtcttcccccgtgacaaagtccttcaacagaggcgagtgtagcccccctagtccaattcctcctcacgtgcagaaatccgtgaacaacgacatgatcgtgaccgacaacaacggcgctgtgaagtttccacagctgtgcaagttctgcgacgtcagattctctacctgtgacaaccagaagtcctgtatgtccaactgctccatcacctctatctgcgagaagccccaagaggtgtgcgtggccgtgtggcggaagaacgatgagaacatcaccctggaaaccgtgtgtcatgatcctaagctgccttaccacgacttcatcctggaagatgccgcttctcccaagtgcatcatgaaagagaagaagaagcctggcgagacattcttcatgtgctcttgtagctccgacgagtgcaacgacaatatcatcttctccgaggaatacaacaccagcaatcctgac

SEQ ID NO: 48 - DNA, 1065 bp, Artificial Sequence
  Synthetic Construct
gatatccagatgacccagtctcctagcagcctgagcgcttctgtgggcgacagagtgacaatcacctgtagagcctctcaggacgtgtccaccgccgtggattggtaccagcagaagcccggcaaggctcctaagctgctgatctactctgcctcctaccggtacacaggagtccccgatagattctctggctccggctctggaaccgacttcaccttcaccatctcctctctgcagcctgaggacattgccacctactactgccagcagcactactccatcccttttaccttcggccagggcaccaagctggaaatcaagcggaccgtggccgctccatccgtgttcatctttcctccttccgacgagcagctgaagtctggcaccgcttccgtggtgtgcctgctgaacaacttctaccctcgggaagccaaggtgcagtggaaagtggacaacgccctgcagtccggcaatagccaagagtccgtcaccgagcaagactccaaggactctacctattctctctccagcacactgaccctgtctaaagccgactacgagaagcacaaggtgtacgcctgcgaagtgacccaccagggcctgtcttcccccgtgacaaagtccttcaacagaggcgagtgtggaccacctggtccaattcctcctcacgtgcagaaatccgtgaacaacgacatgatcgtgaccgacaacaacggcgctgtgaagtttccacagctgtgcaagttctgcgacgtcagattctctacctgtgacaaccagaagtcctgtatgtccaactgctccatcacctctatctgcgagaagccccaagaggtgtgcgtggccgtgtggcggaagaacgatgagaacatcaccctggaaaccgtgtgtcatgatcctaagctgccttaccacgacttcatcctggaagatgccgcttctcccaagtgcatcatgaaagagaagaagaagcctggcgagacattcttcatgtgctcttgtagctccgacgagtgcaacgacaatatcatcttctccgaggaatacaacaccagcaatcctgac

SEQ ID NO: 49 - DNA, 1074 bp, Artificial Sequence
  Synthetic Construct
gatatccagatgacccagtctcctagcagcctgagcgcttctgtgggcgacagagtgacaatcacctgtagagcctctcaggacgtgtccaccgccgtggattggtaccagcagaagcccggcaaggctcctaagctgctgatctactctgcctcctaccggtacacaggagtccccgatagattctctggctccggctctggaaccgacttcaccttcaccatctcctctctgcagcctgaggacattgccacctactactgccagcagcactactccatcccttttaccttcggccagggcaccaagctggaaatcaagcggaccgtggccgctccatccgtgttcatctttcctccttccgacgagcagctgaagtctggcaccgcttccgtggtgtgcctgctgaacaacttctaccctcgggaagccaaggtgcagtggaaagtggacaacgccctgcagtccggcaatagccaagagtccgtcaccgagcaagactccaaggactctacctattctctctccagcacactgaccctgtctaaagccgactacgagaagcacaaggtgtacgcctgcgaagtgacccaccagggcctgtcttcccccgtgacaaagtccttcaacagaggcgagtgtgacaaaactcataccagcccccctattcctcctcacgtgcagaaatccgtgaacaacgacatgatcgtgaccgacaacaacggcgctgtgaagtttccacagctgtgcaagttctgcgacgtcagattctctacctgtgacaaccagaagtcctgtatgtccaactgctccatcacctctatctgcgagaagccccaagaggtgtgcgtggccgtgtggcggaagaacgatgagaacatcaccctggaaaccgtgtgtcatgatcctaagctgccttaccacgacttcatcctggaagatgccgcttctcccaagtgcatcatgaaagagaagaagaagcctggcgagacattcttcatgtgctcttgtagctccgacgagtgcaacgacaatatcatcttctccgaggaatacaacaccagcaatcctgac

SEQ ID NO: 50 - DNA, 1080 bp, Artificial Sequence
  Synthetic Construct
gatatccagatgacccagtctcctagcagcctgagcgcttctgtgggcgacagagtgacaatcacctgtagagcctctcaggacgtgtccaccgccgtggattggtaccagcagaagcccggcaaggctcctaagctgctgatctactctgcctcctaccggtacacaggagtccccgatagattctctggctccggctctggaaccgacttcaccttcaccatctcctctctgcagcctgaggacattgccacctactactgccagcagcactactccatcccttttaccttcggccagggcaccaagctggaaatcaagcggaccgtggccgctccatccgtgttcatctttcctccttccgacgagcagctgaagtctggcaccgcttccgtggtgtgcctgctgaacaacttctaccctcgggaagccaaggtgcagtggaaagtggacaacgccctgcagtccggcaatagccaagagtccgtcaccgagcaagactccaaggactctacctattctctctccagcacactgaccctgtctaaagccgactacgagaagcacaaggtgtacgcctgcgaagtgacccaccagggcctgtcttcccccgtgacaaagtccttcaacagaggcgagtgtgacaaaactcataccagcccccctagtccaattcctcctcacgtgcagaaatccgtgaacaacgacatgatcgtgaccgacaacaacggcgctgtgaagtttccacagctgtgcaagttctgcgacgtcagattctctacctgtgacaaccagaagtcctgtatgtccaactgctccatcacctctatctgcgagaagccccaagaggtgtgcgtggccgtgtggcggaagaacgatgagaacatcaccctggaaaccgtgtgtcatgatcctaagctgccttaccacgacttcatcctggaagatgccgcttctcccaagtgcatcatgaaagagaagaagaagcctggcgagacattcttcatgtgctcttgtagctccgacgagtgcaacgacaatatcatcttctccgaggaatacaacaccagcaatcctgac

SEQ ID NO: 51 - DNA, 1086 bp, Artificial Sequence
  Synthetic Construct
gatatccagatgacccagtctcctagcagcctgagcgcttctgtgggcgacagagtgacaatcacctgtagagcctctcaggacgtgtccaccgccgtggattggtaccagcagaagcccggcaaggctcctaagctgctgatctactctgcctcctaccggtacacaggagtccccgatagattctctggctccggctctggaaccgacttcaccttcaccatctcctctctgcagcctgaggacattgccacctactactgccagcagcactactccatcccttttaccttcggccagggcaccaagctggaaatcaagcggaccgtggccgctccatccgtgttcatctttcctccttccgacgagcagctgaagtctggcaccgcttccgtggtgtgcctgctgaacaacttctaccctcgggaagccaaggtgcagtggaaagtggacaacgccctgcagtccggcaatagccaagagtccgtcaccgagcaagactccaaggactctacctattctctctccagcacactgaccctgtctaaagccgactacgagaagcacaaggtgtacgcctgcgaagtgacccaccagggcctgtcttcccccgtgacaaagtccttcaacagaggcgagtgtgagagcaagtacggaccaccttctccaccatctccaattcctcctcacgtgcagaaatccgtgaacaacgacatgatcgtgaccgacaacaacggcgctgtgaagtttccacagctgtgcaagttctgcgacgtcagattctctacctgtgacaaccagaagtcctgtatgtccaactgctccatcacctctatctgcgagaagccccaagaggtgtgcgtggccgtgtggcggaagaacgatgagaacatcaccctggaaaccgtgtgtcatgatcctaagctgccttaccacgacttcatcctggaagatgccgcttctcccaagtgcatcatgaaagagaagaagaagcctggcgagacattcttcatgtgctcttgtagctccgacgagtgcaacgacaatatcatcttctccgaggaatacaacaccagcaatcctgac

SEQ ID NO: 52 - DNA, 1056 bp, Artificial Sequence
  Synthetic Construct
gatatccagatgacccagtctcctagcagcctgagcgcttctgtgggcgacagagtgacaatcacctgtagagcctctcaggacgtgtccaccgccgtggattggtaccagcagaagcccggcaaggctcctaagctgctgatctactctgcctcctaccggtacacaggagtccccgatagattctctggctccggctctggaaccgacttcaccttcaccatctcctctctgcagcctgaggacattgccacctactactgccagcagcactactccatcccttttaccttcggccagggcaccaagctggaaatcaagcggaccgtggccgctccatccgtgttcatctttcctccttccgacgagcagctgaagtctggcaccgcttccgtggtgtgcctgctgaacaacttctaccctcgggaagccaaggtgcagtggaaagtggacaacgccctgcagtccggcaatagccaagagtccgtcaccgagcaagactccaaggactctacctattctctctccagcacactgaccctgtctaaagccgactacgagaagcacaaggtgtacgcctgcgaagtgacccaccagggcctgtcttcccccgtgacaaagtccttcaacagaggcgagtgtggctcaattcctcctcacgtgcagaaatccgtgaacaacgacatgatcgtgaccgacaacaacggcgctgtgaagtttccacagctgtgcaagttctgcgacgtcagattctctacctgtgacaaccagaagtcctgtatgtccaactgctccatcacctctatctgcgagaagccccaagaggtgtgcgtggccgtgtggcggaagaacgatgagaacatcaccctggaaaccgtgtgtcatgatcctaagctgccttaccacgacttcatcctggaagatgccgcttctcccaagtgcatcatgaaagagaagaagaagcctggcgagacattcttcatgtgctcttgtagctccgacgagtgcaacgacaatatcatcttctccgaggaatacaacaccagcaatcctgac

SEQ ID NO: 53 - DNA, 1062 bp, Artificial Sequence
  Synthetic Construct
gatatccagatgacccagtctcctagcagcctgagcgcttctgtgggcgacagagtgacaatcacctgtagagcctctcaggacgtgtccaccgccgtggattggtaccagcagaagcccggcaaggctcctaagctgctgatctactctgcctcctaccggtacacaggagtccccgatagattctctggctccggctctggaaccgacttcaccttcaccatctcctctctgcagcctgaggacattgccacctactactgccagcagcactactccatcccttttaccttcggccagggcaccaagctggaaatcaagcggaccgtggccgctccatccgtgttcatctttcctccttccgacgagcagctgaagtctggcaccgcttccgtggtgtgcctgctgaacaacttctaccctcgggaagccaaggtgcagtggaaagtggacaacgccctgcagtccggcaatagccaagagtccgtcaccgagcaagactccaaggactctacctattctctctccagcacactgaccctgtctaaagccgactacgagaagcacaaggtgtacgcctgcgaagtgacccaccagggcctgtcttcccccgtgacaaagtccttcaacagaggcgagtgtggtggaggcagtattcctcctcacgtgcagaaatccgtgaacaacgacatgatcgtgaccgacaacaacggcgctgtgaagtttccacagctgtgcaagttctgcgacgtcagattctctacctgtgacaaccagaagtcctgtatgtccaactgctccatcacctctatctgcgagaagccccaagaggtgtgcgtggccgtgtggcggaagaacgatgagaacatcaccctggaaaccgtgtgtcatgatcctaagctgccttaccacgacttcatcctggaagatgccgcttctcccaagtgcatcatgaaagagaagaagaagcctggcgagacattcttcatgtgctcttgtagctccgacgagtgcaacgacaatatcatcttctccgaggaatacaacaccagcaatcctgac

SEQ ID NO: 54 - protein, 19 aa, Artificial Sequence
  Synthetic Construct
MGWSCIILFLVATATGVHS

SEQ ID NO: 55 - DNA, 57 bp, Artificial Sequence
  Synthetic Construct
atgggctggtcctgcatcatcctgttcctggtggctaccgccaccggcgtgcactcc

SEQ ID NO: 56 - protein, 9 aa, Artificial Sequence
  Synthetic Construct
GGSGGGGSG

SEQ ID NO: 57 - protein, 15 aa, Artificial Sequence
  Synthetic Construct
EPKSSDKTHTSPPSP

SEQ ID NO: 58 - protein, 12 aa, Artificial Sequence
  Synthetic Construct
ERKSSVESPPSP

SEQ ID NO: 59 - protein, 21 aa, Artificial Sequence
  Synthetic Construct
GGGGSGGGGSGGGGSGGGGSG

SEQ ID NO: 60 - protein, 15 aa, Artificial Sequence
  Synthetic Construct
GGGSGGGGSGGGGSG

SEQ ID NO: 61 - protein, 4 aa, Artificial Sequence
  Synthetic Construct
GGGS

SEQ ID NO: 62 - protein, 2 aa, Artificial Sequence
  Synthetic Construct
GS

SEQ ID NO: 63 - protein, 4 aa, Artificial Sequence
  Synthetic Construct
SPPS

SEQ ID NO: 64 - protein, 5 aa, Artificial Sequence
  Synthetic Construct
SPPSP

SEQ ID NO: 65 - protein, 5 aa, Artificial Sequence
  Synthetic Construct
GPPGP

SEQ ID NO: 66 - protein, 8 aa, Artificial Sequence
  Synthetic Construct
DKTHTSPP

SEQ ID NO: 67 - protein, 10 aa, Artificial Sequence
  Synthetic Construct
DKTHTSPPSP

SEQ ID NO: 68 - protein, 12 aa, Artificial Sequence
  Synthetic Construct
ESKYGPPSPPSP

SEQ ID NO: 69 - DNA, 27 bp, Artificial Sequence
  Synthetic Construct
ggcggatctggcggaggtggctcaggc

SEQ ID NO: 70 - DNA, 45 bp, Artificial Sequence
  Synthetic Construct
gaacctaagtctagcgacaaaactcataccagcccccctagtcca

SEQ ID NO: 71 - DNA, 36 bp, Artificial Sequence
  Synthetic Construct
gaaaggaagtctagcgtggaatctccacctagtcca

SEQ ID NO: 72 - DNA, 63 bp, Artificial Sequence
  Synthetic Construct
ggtggaggcggtagtggaggcggtggttcaggcggaggcggatctggcggaggtggctcaggc

SEQ ID NO: 73 - DNA, 45 bp, Artificial Sequence
  Synthetic Construct
ggcggtggttcaggcggaggcggatctggcggaggtggctcaggc

SEQ ID NO: 74 - DNA, 12 bp, Artificial Sequence
  Synthetic Construct
ggtggaggcagt

SEQ ID NO: 75 - DNA, 6 bp, Artificial Sequence
  Synthetic Construct
ggctca

SEQ ID NO: 76 - DNA, 12 bp, Artificial Sequence
  Synthetic Construct
agcccccctagt

SEQ ID NO: 77 - DNA, 15 bp, Artificial Sequence
  Synthetic Construct
agcccccctagtcca

SEQ ID NO: 78 - DNA, 15 bp, Artificial Sequence
  Synthetic Construct
ggaccacctggtcca

SEQ ID NO: 79 - DNA, 24 bp, Artificial Sequence
  Synthetic Construct
gacaaaactcataccagcccccct

SEQ ID NO: 80 - DNA, 30 bp, Artificial Sequence
  Synthetic Construct
gacaaaactcataccagcccccctagtcca

SEQ ID NO: 81 - DNA, 36 bp, Artificial Sequence
  Synthetic Construct
gagagcaagtacggaccaccttctccaccatctcca

SEQ ID NO: 82 - protein, 5 aa, Artificial Sequence
  Synthetic Construct
  VARIANT (1)..(5): Can be present in repeats of 2, 3, 4, 5, 6, 7, or 8
GGGGS

SEQ ID NO: 83 - protein, 30 aa, Artificial Sequence
  Synthetic Construct
GGGGSGGGGSGGGGSGGGGSGGGGSGGGGS

SEQ ID NO: 84 - protein, 15 aa, Artificial Sequence
  Synthetic Construct
  VARIANT (1)..(15): Can be present in repeats of 2 or 3
GSTSGSGKPGSGEGS

SEQ ID NO: 85 - protein, 1 aa, Artificial Sequence
  Synthetic Construct
  VARIANT 1: Can be present in repeats of any integer greater than 1
G

SEQ ID NO: 86 - protein, 2 aa, Artificial Sequence
  Synthetic Construct
  VARIANT (1)..(2): Can be present in repeats of any integer greater than 1
GS

SEQ ID NO: 87 - protein, 5 aa, Artificial Sequence
  Synthetic Construct
  VARIANT (1)..(5): Can be present in repeats of 2, 3, 4, 5, 6, 7, or 8
GSGGS

SEQ ID NO: 88 - protein, 4 aa, Artificial Sequence
  Synthetic Construct
  VARIANT (1)..(4): Can be present in repeats of 2, 3, 4, 5, 6, 7, or 8
GGGS

SEQ ID NO: 89 - protein, 79 aa, Artificial Sequence
  Synthetic Construct
ALQCFCHLCTKDNFTCVTDGLCFVSVTETTDKVIHNSMCIAEIDLIPRDRPFVCAPSSKTGSVTTTYCCNQDHCNKIEL

SEQ ID NO: 90 - protein, 101 aa, Artificial Sequence
  Synthetic Construct
AALLPGATALQCFCHLCTKDNFTCVTDGLCFVSVTETTDKVIHNSMCIAEIDLIPRDRPFVCAPSSKTGSVTTTYCCNQDHCNKIELPTTVKSSPGLGPVE

SEQ ID NO: 91 - protein, 101 aa, Artificial Sequence
  Synthetic Construct
QLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIF

SEQ ID NO: 92 - protein, 126 aa, Artificial Sequence
  Synthetic Construct
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIF

SEQ ID NO: 93 - protein, 111 aa, Artificial Sequence
  Synthetic Construct
QLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD